SEQUENCE LISTING (793 sequences)

SEQ ID NO: 1 - protein, 4 aa, Artificial Sequence
  synthetic peptide
  misc_feature (1)..(1): Xaa can be any naturally occurring amino acid
  PEPTIDE (2)..(2): C, S, or T
  misc_feature (3)..(3): Xaa can be any naturally occurring amino acid
XXXC

SEQ ID NO: 2 - protein, 4 aa, Artificial Sequence
  synthetic peptide
  misc_feature (2)..(3): Xaa can be any naturally occurring amino acid
  PEPTIDE (4)..(4): C, S, or T
CXXX

SEQ ID NO: 3 - protein, 9 aa, Artificial Sequence
  synthetic peptide
LALEGSLQK

SEQ ID NO: 4 - protein, 9 aa, Artificial Sequence
  synthetic peptide
FLRVPCWKI

SEQ ID NO: 5 - protein, 9 aa, Artificial Sequence
  synthetic peptide
FLRVPSWKI

SEQ ID NO: 6 - protein, 4 aa, Artificial Sequence
  synthetic peptide
KKCC

SEQ ID NO: 7 - protein, 4 aa, Artificial Sequence
  synthetic peptide
RRCC

SEQ ID NO: 8 - protein, 4 aa, Artificial Sequence
  synthetic peptide
RKCC

SEQ ID NO: 9 - protein, 4 aa, Artificial Sequence
  synthetic peptide
KRCC

SEQ ID NO: 10 - protein, 4 aa, Artificial Sequence
  synthetic peptide
  misc_feature (3)..(3): Xaa can be any naturally occurring amino acid
KCXC

SEQ ID NO: 11 - protein, 5 aa, Artificial Sequence
  synthetic peptide
  misc_feature (4)..(4): Xaa can be any naturally occurring amino acid
KKCXC

SEQ ID NO: 12 - protein, 4 aa, Artificial Sequence
  synthetic peptide
  misc_feature (3)..(3): Xaa can be any naturally occurring amino acid
RCXC

SEQ ID NO: 13 - protein, 5 aa, Artificial Sequence
  synthetic peptide
  misc_feature (4)..(4): Xaa can be any naturally occurring amino acid
RRCXC

SEQ ID NO: 14 - protein, 5 aa, Artificial Sequence
  synthetic peptide
  misc_feature (4)..(4): Xaa can be any naturally occurring amino acid
RKCXC

SEQ ID NO: 15 - protein, 5 aa, Artificial Sequence
  synthetic peptide
  misc_feature (4)..(4): Xaa can be any naturally occurring amino acid
KRCXC

SEQ ID NO: 16 - protein, 4 aa, Artificial Sequence
  synthetic peptide
KCKC

SEQ ID NO: 17 - protein, 5 aa, Artificial Sequence
  synthetic peptide
KKCKC

SEQ ID NO: 18 - protein, 4 aa, Artificial Sequence
  synthetic peptide
KCRC

SEQ ID NO: 19 - protein, 5 aa, Artificial Sequence
  synthetic peptide
KKCRC

SEQ ID NO: 20 - protein, 4 aa, Artificial Sequence
  synthetic peptide
RCRC

SEQ ID NO: 21 - protein, 5 aa, Artificial Sequence
  synthetic peptide
RRCRC

SEQ ID NO: 22 - protein, 5 aa, Artificial Sequence
  synthetic peptide
RKCKC

SEQ ID NO: 23 - protein, 5 aa, Artificial Sequence
  synthetic peptide
KRCKC

SEQ ID NO: 24 - protein, 5 aa, Artificial Sequence
  synhtetic peptide
HCPYC

SEQ ID NO: 25 - protein, 6 aa, Artificial Sequence
  synthetic peptide
KHCPYC

SEQ ID NO: 26 - protein, 5 aa, Artificial Sequence
  synthetic peptide
KCPYC

SEQ ID NO: 27 - protein, 5 aa, Artificial Sequence
  synthetic peptide
RCPYC

SEQ ID NO: 28 - protein, 5 aa, Artificial Sequence
  synthetic peptide
HCGHC

SEQ ID NO: 29 - protein, 5 aa, Artificial Sequence
  synthetic peptide
KCGHC

SEQ ID NO: 30 - protein, 5 aa, Artificial Sequence
  synthetic peptide
RCGHC

SEQ ID NO: 31 - protein, 6 aa, Artificial Sequence
  synthetic peptide
KKCPYC

SEQ ID NO: 32 - protein, 6 aa, Artificial Sequence
  synthetic peptide
KRCPYC

SEQ ID NO: 33 - protein, 6 aa, Artificial Sequence
  synthetic peptide
KHCGHC

SEQ ID NO: 34 - protein, 6 aa, Artificial Sequence
  synthetic peptide
KKCGHC

SEQ ID NO: 35 - protein, 6 aa, Artificial Sequence
  synthetic peptide
KRCGHC

SEQ ID NO: 36 - protein, 5 aa, Artificial Sequence
  synthetic peptide
CRPYC

SEQ ID NO: 37 - protein, 6 aa, Artificial Sequence
  synthetic peptide
KCRPYC

SEQ ID NO: 38 - protein, 7 aa, Artificial Sequence
  synthetic peptide
KHCRPYC

SEQ ID NO: 39 - protein, 6 aa, Artificial Sequence
  synthetic peptide
RCRPYC

SEQ ID NO: 40 - protein, 6 aa, Artificial Sequence
  synthetic peptide
HCRPYC

SEQ ID NO: 41 - protein, 5 aa, Artificial Sequence
  synthetic peptide
CPRYC

SEQ ID NO: 42 - protein, 6 aa, Artificial Sequence
  synthetic peptide
KCPRYC

SEQ ID NO: 43 - protein, 6 aa, Artificial Sequence
  synthetic peptide
RCPRYC

SEQ ID NO: 44 - protein, 6 aa, Artificial Sequence
  synthetic peptide
HCPRYC

SEQ ID NO: 45 - protein, 5 aa, Artificial Sequence
  synthetic peptide
CPYRC

SEQ ID NO: 46 - protein, 6 aa, Artificial Sequence
  synthetic peptide
KCPYRC

SEQ ID NO: 47 - protein, 6 aa, Artificial Sequence
  synthetic peptide
RCPYRC

SEQ ID NO: 48 - protein, 6 aa, Artificial Sequence
  synthetic peptide
HCPYRC

SEQ ID NO: 49 - protein, 5 aa, Artificial Sequence
  synthetic peptide
CKPYC

SEQ ID NO: 50 - protein, 6 aa, Artificial Sequence
  synthetic peptide
KCKPYC

SEQ ID NO: 51 - protein, 6 aa, Artificial Sequence
  synthetic peptide
RCKPYC

SEQ ID NO: 52 - protein, 6 aa, Artificial Sequence
  synthetic peptide
HCKPYC

SEQ ID NO: 53 - protein, 5 aa, Artificial Sequence
  synthetic peptide
CPKYC

SEQ ID NO: 54 - protein, 6 aa, Artificial Sequence
  synthetic peptide
KCPKYC

SEQ ID NO: 55 - protein, 6 aa, Artificial Sequence
  synthetic peptide
RCPKYC

SEQ ID NO: 56 - protein, 6 aa, Artificial Sequence
  synthetic peptide
HCPKYC

SEQ ID NO: 57 - protein, 5 aa, Artificial Sequence
  synthetic peptide
CPYKC

SEQ ID NO: 58 - protein, 6 aa, Artificial Sequence
  synthetic peptide
KCPYKC

SEQ ID NO: 59 - protein, 6 aa, Artificial Sequence
  synthetic peptide
RCPYKC

SEQ ID NO: 60 - protein, 6 aa, Artificial Sequence
  synthetic peptide
HCPYKC

SEQ ID NO: 61 - protein, 4 aa, Artificial Sequence
  synthetic peptide
LAVL

SEQ ID NO: 62 - protein, 4 aa, Artificial Sequence
  synthetic peptide
TVQA

SEQ ID NO: 63 - protein, 4 aa, Artificial Sequence
  synthetic peptide
GAVH

SEQ ID NO: 64 - protein, 4 aa, Artificial Sequence
  synthetic peptide
  PEPTIDE (1)..(1): G, A, V, L, I, M, F, W, P, S, T, C, Y, N, Q, D, E, K, R, and H, or non-natural basic amino acid
XAVL

SEQ ID NO: 65 - protein, 4 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
LXVL

SEQ ID NO: 66 - protein, 4 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
LAXL

SEQ ID NO: 67 - protein, 4 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
LAVX

SEQ ID NO: 68 - protein, 4 aa, Artificial Sequence
  synthetic peptide
  PEPTIDE (1)..(1): G, A, V, L, I, M, F, W, P, S, T, C, Y, N, Q, D, E, K, R, and H, or non-natural basic amino acid
XVQA

SEQ ID NO: 69 - protein, 4 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
TXQA

SEQ ID NO: 70 - protein, 4 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
TVXA

SEQ ID NO: 71 - protein, 4 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
TVQX

SEQ ID NO: 72 - protein, 4 aa, Artificial Sequence
  synthetic peptide
  PEPTIDE (1)..(1): G, A, V, L, I, M, F, W, P, S, T, C, Y, N, Q, D, E, K, R, and H, or non-natural basic amino acid
XAVH

SEQ ID NO: 73 - protein, 4 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
GXVH

SEQ ID NO: 74 - protein, 4 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
GAXH

SEQ ID NO: 75 - protein, 4 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
GAVX

SEQ ID NO: 76 - protein, 4 aa, Artificial Sequence
  synthetic peptide
  PEPTIDE (1)..(4): G, A, V, L, I, M, F, W, P, S, T, C, Y, N, Q, D, E, K, R, and H, or non-natural amino acid
XXXX

SEQ ID NO: 77 - protein, 5 aa, Artificial Sequence
  synthetic peptide
  PEPTIDE (1)..(4): G, A, V, L, I, M, F, W, P, S, T, C, Y, N, Q, D, E, K, R, and H, or non-natural amino acid
  misc_feature (5)..(5): Xaa can be any naturally occurring amino acid
XXXXX

SEQ ID NO: 78 - protein, 5 aa, Artificial Sequence
  synthetic peptide
PAFPL

SEQ ID NO: 79 - protein, 5 aa, Artificial Sequence
  synthetic peptide
DQGGE

SEQ ID NO: 80 - protein, 5 aa, Artificial Sequence
  synthetic peptide
  PEPTIDE (1)..(1): G, A, V, L, I, M, F, W, P, S, T, C, Y, N, Q, D, E, K, R, and H, or non-natural amino acid
XAFPL

SEQ ID NO: 81 - protein, 5 aa, Artificial Sequence
  synthetic peptide
  PEPTIDE (2)..(2): G, A, V, L, I, M, F, W, P, S, T, C, Y, N, Q, D, E, K, R, and H, or non-natural amino acid
PXFPL

SEQ ID NO: 82 - protein, 5 aa, Artificial Sequence
  synthetic peptide
  PEPTIDE (3)..(3): G, A, V, L, I, M, F, W, P, S, T, C, Y, N, Q, D, E, K, R, and H, or non-natural amino acid
PAXPL

SEQ ID NO: 83 - protein, 5 aa, Artificial Sequence
  synthetic peptide
  PEPTIDE (4)..(4): G, A, V, L, I, M, F, W, P, S, T, C, Y, N, Q, D, E, K, R, and H, or non-natural amino acid
PAFXL

SEQ ID NO: 84 - protein, 5 aa, Artificial Sequence
  synthetic peptide
  PEPTIDE (5)..(5): G, A, V, L, I, M, F, W, P, S, T, C, Y, N, Q, D, E, K, R, and H, or non-natural amino acid
PAFPX

SEQ ID NO: 85 - protein, 5 aa, Artificial Sequence
  synthetic peptide
  PEPTIDE (1)..(1): G, A, V, L, I, M, F, W, P, S, T, C, Y, N, Q, D, E, K, R, and H, or non-natural amino acid
XQGGE

SEQ ID NO: 86 - protein, 5 aa, Artificial Sequence
  synthetic peptide
  PEPTIDE (2)..(2): G, A, V, L, I, M, F, W, P, S, T, C, Y, N, Q, D, E, K, R, and H, or non-natural amino acid
DXGGE

SEQ ID NO: 87 - protein, 5 aa, Artificial Sequence
  synthetic peptide
  PEPTIDE (3)..(3): G, A, V, L, I, M, F, W, P, S, T, C, Y, N, Q, D, E, K, R, and H, or non-natural amino acid
DQXGE

SEQ ID NO: 88 - protein, 5 aa, Artificial Sequence
  synthetic peptide
  PEPTIDE (4)..(4): G, A, V, L, I, M, F, W, P, S, T, C, Y, N, Q, D, E, K, R, and H, or non-natural amino acid
DQGXE

SEQ ID NO: 89 - protein, 5 aa, Artificial Sequence
  synthetic peptide
  PEPTIDE (5)..(5): G, A, V, L, I, M, F, W, P, S, T, C, Y, N, Q, D, E, K, R, and H, or non-natural amino acid
DQGGX

SEQ ID NO: 90 - protein, 6 aa, Artificial Sequence
  synthetic peptide
  PEPTIDE (1)..(6): G, A, V, L, I, M, F, W, P, S, T, C, Y, N, Q, D, E, K, R, and H, or non-natural amino acid
XXXXXX

SEQ ID NO: 91 - protein, 6 aa, Artificial Sequence
  synthetic peptide
DIADKY

SEQ ID NO: 92 - protein, 6 aa, Artificial Sequence
  synthetic peptide
  PEPTIDE (1)..(1): G, A, V, L, I, M, F, W, P, S, T, C, Y, N, Q, D, E, K, R, and H, or non-natural basic amino acid
XIADKY

SEQ ID NO: 93 - protein, 6 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
DXADKY

SEQ ID NO: 94 - protein, 6 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
DIXDKY

SEQ ID NO: 95 - protein, 6 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
DIAXKY

SEQ ID NO: 96 - protein, 6 aa, Artificial Sequence
  synthetic peptide
  PEPTIDE (5)..(5): G, A, V, L, I, M, F, W, P, S, T, C, Y, N, Q, D, E, K, R, and H, or non-natural basic amino acid
DIADXY

SEQ ID NO: 97 - protein, 6 aa, Artificial Sequence
  synthetic peptide
  PEPTIDE (6)..(6): G, A, V, L, I, M, F, W, P, S, T, C, Y, N, Q, D, E, K, R, and H, or non-natural basic amino acid
DIADKX

SEQ ID NO: 98 - protein, 4 aa, Artificial Sequence
  synthetic peptide
CPYC

SEQ ID NO: 99 - protein, 14 aa, Artificial Sequence
  synthetic peptide
  misc_feature (2)..(3): Xaa can be any naturally occurring amino acid
  misc_feature (5)..(5): Xaa can be any naturally occurring amino acid
  misc_feature (7)..(8): Xaa can be any naturally occurring amino acid
  misc_feature (10)..(10): Xaa can be any naturally occurring amino acid
  misc_feature (12)..(13): Xaa can be any naturally occurring amino acid
CXXCXCXXCXCXXC

SEQ ID NO: 100 - protein, 12 aa, Artificial Sequence
  synthetic peptide
  misc_feature (2)..(3): Xaa can be any naturally occurring amino acid
  misc_feature (6)..(7): Xaa can be any naturally occurring amino acid
  misc_feature (10)..(11): Xaa can be any naturally occurring amino acid
CXXCCXXCCXXC

SEQ ID NO: 101 - protein, 10 aa, Artificial Sequence
  synthetic peptide
  misc_feature (2)..(3): Xaa can be any naturally occurring amino acid
  misc_feature (5)..(6): Xaa can be any naturally occurring amino acid
  misc_feature (8)..(9): Xaa can be any naturally occurring amino acid
CXXCXXCXXC

SEQ ID NO: 102 - protein, 10 aa, Artificial Sequence
  synthetic peptide
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
CXCCXCCXCC

SEQ ID NO: 103 - protein, 7 aa, Artificial Sequence
  synthetic peptide
  PEPTIDE (1)..(1): F, W, Y, H, or T
  misc_feature (2)..(3): Xaa can be any naturally occurring amino acid
  PEPTIDE (4)..(4): V, I, L, or M
  misc_feature (5)..(6): Xaa can be any naturally occurring amino acid
  PEPTIDE (7)..(7): F, W, Y, H, or T
XXXXXXX

SEQ ID NO: 104 - protein, 7 aa, Artificial Sequence
  synthetic peptide
  PEPTIDE (1)..(1): F, W, Y, or H
  misc_feature (2)..(3): Xaa can be any naturally occurring amino acid
  PEPTIDE (4)..(4): V, I, L, or M
  misc_feature (5)..(6): Xaa can be any naturally occurring amino acid
  PEPTIDE (7)..(7): F, W, Y, or H
XXXXXXX

SEQ ID NO: 105 - protein, 7 aa, Artificial Sequence
  synthetic peptide
  PEPTIDE (1)..(1): F, W, Y, or T
  misc_feature (2)..(3): Xaa can be any naturally occurring amino acid
  PEPTIDE (4)..(4): V, I, L, or M
  misc_feature (5)..(6): Xaa can be any naturally occurring amino acid
  PEPTIDE (7)..(7): F, W, Y, or T
XXXXXXX

SEQ ID NO: 106 - protein, 7 aa, Artificial Sequence
  synthetic peptide
  PEPTIDE (1)..(1): F, W, or Y
  misc_feature (2)..(3): Xaa can be any naturally occurring amino acid
  PEPTIDE (4)..(4): V, I, L, or M
  misc_feature (5)..(6): Xaa can be any naturally occurring amino acid
  PEPTIDE (7)..(7): F, W, or Y
XXXXXXX

SEQ ID NO: 107 - protein, 7 aa, Artificial Sequence
  synthetic peptide
  PEPTIDE (1)..(1): F, W, Y, or H
  misc_feature (2)..(3): Xaa can be any naturally occurring amino acid
  PEPTIDE (4)..(4): I, L, or M
  misc_feature (5)..(6): Xaa can be any naturally occurring amino acid
  PEPTIDE (7)..(7): F, W, Y, or H
XXXXXXX

SEQ ID NO: 108 - protein, 7 aa, Artificial Sequence
  synthetic peptide
  PEPTIDE (1)..(1): F, W, Y, H, or T
  misc_feature (2)..(3): Xaa can be any naturally occurring amino acid
  PEPTIDE (4)..(4): I, L, or M
  misc_feature (5)..(6): Xaa can be any naturally occurring amino acid
  PEPTIDE (7)..(7): F, W, Y, H, or T
XXXXXXX

SEQ ID NO: 109 - protein, 7 aa, Artificial Sequence
  synthetic peptide
  PEPTIDE (1)..(1): F, W, or Y
  misc_feature (2)..(3): Xaa can be any naturally occurring amino acid
  PEPTIDE (4)..(4): I, L, or M
  misc_feature (5)..(6): Xaa can be any naturally occurring amino acid
  PEPTIDE (7)..(7): F, W, or Y
XXXXXXX

SEQ ID NO: 110 - protein, 6 aa, Artificial Sequence
  synthetic peptide
  PEPTIDE (1)..(1): D or E
  misc_feature (2)..(4): Xaa can be any naturally occurring amino acid
  PEPTIDE (6)..(6): L or I
XXXXLX

SEQ ID NO: 111 - protein, 5 aa, Artificial Sequence
  synthetic peptide
  misc_feature (2)..(3): Xaa can be any naturally occurring amino acid
DXXLL

SEQ ID NO: 112 - protein, 6 aa, Artificial Sequence
  synthetic peptide
  misc_feature (2)..(4): Xaa can be any naturally occurring amino acid
DXXXLL

SEQ ID NO: 113 - protein, 4 aa, Artificial Sequence
  synthetic peptide
  misc_feature (2)..(3): Xaa can be any naturally occurring amino acid
  PEPTIDE (4)..(4): amino acid residues with a bulky hydrophobic side chain such as Phe, Tyr and Trp
YXXX

SEQ ID NO: 114 - protein, 4 aa, Artificial Sequence
  synthetic peptide
KCHC

SEQ ID NO: 115 - protein, 4 aa, Artificial Sequence
  synthetic peptide
KCKC

SEQ ID NO: 116 - protein, 4 aa, Artificial Sequence
  synthetic peptide
KCRC

SEQ ID NO: 117 - protein, 4 aa, Artificial Sequence
  synthetic peptide
KCGC

SEQ ID NO: 118 - protein, 4 aa, Artificial Sequence
  synthetic peptide
KCAC

SEQ ID NO: 119 - protein, 4 aa, Artificial Sequence
  synthetic peptide
KCVC

SEQ ID NO: 120 - protein, 4 aa, Artificial Sequence
  synthetic peptide
KCLC

SEQ ID NO: 121 - protein, 4 aa, Artificial Sequence
  synthetic peptide
KCIC

SEQ ID NO: 122 - protein, 4 aa, Artificial Sequence
  synthetic peptide
KCMC

SEQ ID NO: 123 - protein, 4 aa, Artificial Sequence
  synthetic peptide
KCFC

SEQ ID NO: 124 - protein, 4 aa, Artificial Sequence
  synthetic peptide
KCWC

SEQ ID NO: 125 - protein, 4 aa, Artificial Sequence
  synthetic peptide
KCPC

SEQ ID NO: 126 - protein, 4 aa, Artificial Sequence
  synthetic peptide
KCSC

SEQ ID NO: 127 - protein, 4 aa, Artificial Sequence
  synthetic peptide
KCTC

SEQ ID NO: 128 - protein, 4 aa, Artificial Sequence
  synthetic peptide
KCYC

SEQ ID NO: 129 - protein, 4 aa, Artificial Sequence
  synthetic peptide
KCNC

SEQ ID NO: 130 - protein, 4 aa, Artificial Sequence
  synthetic peptide
KCQC

SEQ ID NO: 131 - protein, 4 aa, Artificial Sequence
  synthetic peptide
KCDC

SEQ ID NO: 132 - protein, 4 aa, Artificial Sequence
  synthetic peptide
KCEC

SEQ ID NO: 133 - protein, 4 aa, Artificial Sequence
  synthetic peptide
HCHC

SEQ ID NO: 134 - protein, 4 aa, Artificial Sequence
  synthetic peptide
HCKC

SEQ ID NO: 135 - protein, 4 aa, Artificial Sequence
  synthetic peptide
HCRC

SEQ ID NO: 136 - protein, 4 aa, Artificial Sequence
  synthetic peptide
HCGC

SEQ ID NO: 137 - protein, 4 aa, Artificial Sequence
  synthetic peptide
HCAC

SEQ ID NO: 138 - protein, 4 aa, Artificial Sequence
  synthetic peptide
HCVC

SEQ ID NO: 139 - protein, 4 aa, Artificial Sequence
  synthetic peptide
HCLC

SEQ ID NO: 140 - protein, 4 aa, Artificial Sequence
  synthetic peptide
HCIC

SEQ ID NO: 141 - protein, 4 aa, Artificial Sequence
  synthetic peptide
HCMC

SEQ ID NO: 142 - protein, 4 aa, Artificial Sequence
  synthetic peptide
HCFC

SEQ ID NO: 143 - protein, 4 aa, Artificial Sequence
  synthetic peptide
HCWC

SEQ ID NO: 144 - protein, 4 aa, Artificial Sequence
  synthetic peptide
HCPC

SEQ ID NO: 145 - protein, 4 aa, Artificial Sequence
  synthetic peptide
HCSC

SEQ ID NO: 146 - protein, 4 aa, Artificial Sequence
  synthetic peptide
HCTC

SEQ ID NO: 147 - protein, 4 aa, Artificial Sequence
  synthetic peptide
HCYC

SEQ ID NO: 148 - protein, 4 aa, Artificial Sequence
  synthetic peptide
HCNC

SEQ ID NO: 149 - protein, 4 aa, Artificial Sequence
  synthetic peptide
HCQC

SEQ ID NO: 150 - protein, 4 aa, Artificial Sequence
  synthetic peptide
HCDC

SEQ ID NO: 151 - protein, 4 aa, Artificial Sequence
  synthetic peptide
HCEC

SEQ ID NO: 152 - protein, 4 aa, Artificial Sequence
  synthetic peptide
RCHC

SEQ ID NO: 153 - protein, 4 aa, Artificial Sequence
  synthetic peptide
RCKC

SEQ ID NO: 154 - protein, 4 aa, Artificial Sequence
  synthetic peptide
RCRC

SEQ ID NO: 155 - protein, 4 aa, Artificial Sequence
  synthetic peptide
RCGC

SEQ ID NO: 156 - protein, 4 aa, Artificial Sequence
  synthetic peptide
RCAC

SEQ ID NO: 157 - protein, 4 aa, Artificial Sequence
  synthetic peptide
RCVC

SEQ ID NO: 158 - protein, 4 aa, Artificial Sequence
  synthetic peptide
RCLC

SEQ ID NO: 159 - protein, 4 aa, Artificial Sequence
  synthetic peptide
RCIC

SEQ ID NO: 160 - protein, 4 aa, Artificial Sequence
  synthetic peptide
RCMC

SEQ ID NO: 161 - protein, 4 aa, Artificial Sequence
  synthetic peptide
RCFC

SEQ ID NO: 162 - protein, 4 aa, Artificial Sequence
  synthetic peptide
RCWC

SEQ ID NO: 163 - protein, 4 aa, Artificial Sequence
  synthetic peptide
RCPC

SEQ ID NO: 164 - protein, 4 aa, Artificial Sequence
  synthetic peptide
RCSC

SEQ ID NO: 165 - protein, 4 aa, Artificial Sequence
  synthetic peptide
RCTC

SEQ ID NO: 166 - protein, 4 aa, Artificial Sequence
  synthetic peptide
RCYC

SEQ ID NO: 167 - protein, 4 aa, Artificial Sequence
  synthetic peptide
RCNC

SEQ ID NO: 168 - protein, 4 aa, Artificial Sequence
  synthetic peptide
RCQC

SEQ ID NO: 169 - protein, 4 aa, Artificial Sequence
  synthetic peptide
RCDC

SEQ ID NO: 170 - protein, 4 aa, Artificial Sequence
  synthetic peptide
RCEC

SEQ ID NO: 171 - protein, 4 aa, Artificial Sequence
  synthetic peptide
  PEPTIDE (1)..(2): G, A, V, L, I, M, F, W, P, S, T, C, Y, N, Q, D, E, K, R, and H, or non-natural basic amino acid
  misc_feature (3)..(3): Xaa can be any naturally occurring amino acid
CXXC

SEQ ID NO: 172 - protein, 5 aa, Artificial Sequence
  synthetic peptide
  PEPTIDE (3)..(4): G, A, V, L, I, M, F, W, P, S, T, C, Y, N, Q, D, E, K, R, and H, or non-natural basic amino acid
HCXXC

SEQ ID NO: 173 - protein, 6 aa, Artificial Sequence
  synthetic peptide
  PEPTIDE (4)..(5): G, A, V, L, I, M, F, W, P, S, T, C, Y, N, Q, D, E, K, R, and H, or non-natural basic amino acid
KHCXXC

SEQ ID NO: 174 - protein, 5 aa, Artificial Sequence
  synthetic peptide
  PEPTIDE (3)..(4): G, A, V, L, I, M, F, W, P, S, T, C, Y, N, Q, D, E, K, R, and H, or non-natural basic amino acid
KCXXC

SEQ ID NO: 175 - protein, 5 aa, Artificial Sequence
  synthetic peptide
  PEPTIDE (3)..(4): G, A, V, L, I, M, F, W, P, S, T, C, Y, N, Q, D, E, K, R, and H, or non-natural basic amino acid
RCXXC

SEQ ID NO: 176 - protein, 5 aa, Artificial Sequence
  synthetic peptide
  PEPTIDE (3)..(4): G, A, V, L, I, M, F, W, P, S, T, C, Y, N, Q, D, E, K, R, and H, or non-natural basic amino acid
HCXXC

SEQ ID NO: 177 - protein, 5 aa, Artificial Sequence
  synthetic peptide
  PEPTIDE (3)..(4): G, A, V, L, I, M, F, W, P, S, T, C, Y, N, Q, D, E, K, R, and H, or non-natural basic amino acid
KCXXC

SEQ ID NO: 178 - protein, 5 aa, Artificial Sequence
  synthetic peptide
  PEPTIDE (3)..(4): G, A, V, L, I, M, F, W, P, S, T, C, Y, N, Q, D, E, K, R, and H, or non-natural basic amino acid
RCXXC

SEQ ID NO: 179 - protein, 6 aa, Artificial Sequence
  synthetic peptide
  PEPTIDE (4)..(5): G, A, V, L, I, M, F, W, P, S, T, C, Y, N, Q, D, E, K, R, and H, or non-natural basic amino acid
KKCXXC

SEQ ID NO: 180 - protein, 6 aa, Artificial Sequence
  synthetic peptide
  PEPTIDE (4)..(5): G, A, V, L, I, M, F, W, P, S, T, C, Y, N, Q, D, E, K, R, and H, or non-natural basic amino acid
KRCXXC

SEQ ID NO: 181 - protein, 6 aa, Artificial Sequence
  synthetic peptide
  PEPTIDE (4)..(5): G, A, V, L, I, M, F, W, P, S, T, C, Y, N, Q, D, E, K, R, and H, or non-natural basic amino acid
KHCXXC

SEQ ID NO: 182 - protein, 6 aa, Artificial Sequence
  synthetic peptide
  PEPTIDE (4)..(5): G, A, V, L, I, M, F, W, P, S, T, C, Y, N, Q, D, E, K, R, and H, or non-natural basic amino acid
KKCXXC

SEQ ID NO: 183 - protein, 6 aa, Artificial Sequence
  synthetic peptide
  PEPTIDE (4)..(5): G, A, V, L, I, M, F, W, P, S, T, C, Y, N, Q, D, E, K, R, and H, or non-natural basic amino acid
KRCXXC

SEQ ID NO: 184 - protein, 5 aa, Artificial Sequence
  synthetic peptide
  PEPTIDE (2)..(4): G, A, V, L, I, M, F, W, P, S, T, C, Y, N, Q, D, E, K, R, and H, or non-natural basic amino acid
CXXXC

SEQ ID NO: 185 - protein, 5 aa, Artificial Sequence
  synthetic peptide
  misc_feature (2)..(2): Xaa can be any naturally occurring amino acid
CXPYC

SEQ ID NO: 186 - protein, 5 aa, Artificial Sequence
  synthetic peptide
CKPYC

SEQ ID NO: 187 - protein, 5 aa, Artificial Sequence
  synthetic peptide
CRPYC

SEQ ID NO: 188 - protein, 5 aa, Artificial Sequence
  synthetic peptide
CHPYC

SEQ ID NO: 189 - protein, 5 aa, Artificial Sequence
  synthetic peptide
CGPYC

SEQ ID NO: 190 - protein, 5 aa, Artificial Sequence
  synthetic peptide
CAPYC

SEQ ID NO: 191 - protein, 5 aa, Artificial Sequence
  synthetic peptide
CVPYC

SEQ ID NO: 192 - protein, 5 aa, Artificial Sequence
  synthetic peptide
CLPYC

SEQ ID NO: 193 - protein, 5 aa, Artificial Sequence
  synthetic peptide
CIPYC

SEQ ID NO: 194 - protein, 5 aa, Artificial Sequence
  synthetic peptide
CMPYC

SEQ ID NO: 195 - protein, 5 aa, Artificial Sequence
  synthetic peptide
CFPYC

SEQ ID NO: 196 - protein, 5 aa, Artificial Sequence
  synthetic peptide
CWPYC

SEQ ID NO: 197 - protein, 5 aa, Artificial Sequence
  synthetic peptide
CPPYC

SEQ ID NO: 198 - protein, 5 aa, Artificial Sequence
  synthetic peptide
CSPYC

SEQ ID NO: 199 - protein, 5 aa, Artificial Sequence
  synthetic peptide
CTPYC

SEQ ID NO: 200 - protein, 5 aa, Artificial Sequence
  synthetic peptide
CCPYC

SEQ ID NO: 201 - protein, 5 aa, Artificial Sequence
  synthetic peptide
CYPYC

SEQ ID NO: 202 - protein, 5 aa, Artificial Sequence
  synthetic peptide
CNPYC

SEQ ID NO: 203 - protein, 5 aa, Artificial Sequence
  synthetic peptide
CQPYC

SEQ ID NO: 204 - protein, 5 aa, Artificial Sequence
  synthetic peptide
CDPYC

SEQ ID NO: 205 - protein, 5 aa, Artificial Sequence
  synthetic peptide
CEPYC

SEQ ID NO: 206 - protein, 5 aa, Artificial Sequence
  synthetic peptide
  misc_feature (3)..(3): Xaa can be any naturally occurring amino acid
CPXYC

SEQ ID NO: 207 - protein, 5 aa, Artificial Sequence
  synthetic peptide
CPKYC

SEQ ID NO: 208 - protein, 5 aa, Artificial Sequence
  synthetic peptide
CPRYC

SEQ ID NO: 209 - protein, 5 aa, Artificial Sequence
  synthetic peptide
CPHYC

SEQ ID NO: 210 - protein, 5 aa, Artificial Sequence
  synthetic peptide
CPGYC

SEQ ID NO: 211 - protein, 5 aa, Artificial Sequence
  synthetic peptide
CPAYC

SEQ ID NO: 212 - protein, 5 aa, Artificial Sequence
  synthetic peptide
CPVYC

SEQ ID NO: 213 - protein, 5 aa, Artificial Sequence
  synthetic peptide
CPLYC

SEQ ID NO: 214 - protein, 5 aa, Artificial Sequence
  synthetic peptide
CPIYC

SEQ ID NO: 215 - protein, 5 aa, Artificial Sequence
  synthetic peptide
CPMYC

SEQ ID NO: 216 - protein, 5 aa, Artificial Sequence
  synthetic peptide
CPFYC

SEQ ID NO: 217 - protein, 5 aa, Artificial Sequence
  synthetic peptide
CPWYC

SEQ ID NO: 218 - protein, 5 aa, Artificial Sequence
  synthetic peptide
CPPYC

SEQ ID NO: 219 - protein, 5 aa, Artificial Sequence
  synthetic peptide
CPSYC

SEQ ID NO: 220 - protein, 5 aa, Artificial Sequence
  synthetic peptide
CPTYC

SEQ ID NO: 221 - protein, 5 aa, Artificial Sequence
  synthetic peptide
CPCYC

SEQ ID NO: 222 - protein, 5 aa, Artificial Sequence
  synthetic peptide
CPYYC

SEQ ID NO: 223 - protein, 5 aa, Artificial Sequence
  synthetic peptide
CPNYC

SEQ ID NO: 224 - protein, 5 aa, Artificial Sequence
  synthetic peptide
CPQYC

SEQ ID NO: 225 - protein, 5 aa, Artificial Sequence
  synthetic peptide
CPDYC

SEQ ID NO: 226 - protein, 5 aa, Artificial Sequence
  synthetic peptide
CPEYC

SEQ ID NO: 227 - protein, 5 aa, Artificial Sequence
  synthetic peptide
CPLYC

SEQ ID NO: 228 - protein, 5 aa, Artificial Sequence
  synthetic peptide
  misc_feature (4)..(4): Xaa can be any naturally occurring amino acid
CPYXC

SEQ ID NO: 229 - protein, 5 aa, Artificial Sequence
  synthetic peptide
CPYKC

SEQ ID NO: 230 - protein, 5 aa, Artificial Sequence
  synthetic peptide
CPYRC

SEQ ID NO: 231 - protein, 5 aa, Artificial Sequence
  synthetic peptide
CPYHC

SEQ ID NO: 232 - protein, 5 aa, Artificial Sequence
  synthetic peptide
CPYGC

SEQ ID NO: 233 - protein, 5 aa, Artificial Sequence
  synthetic peptide
CPYAC

SEQ ID NO: 234 - protein, 5 aa, Artificial Sequence
  synthetic peptide
CPYVC

SEQ ID NO: 235 - protein, 5 aa, Artificial Sequence
  synthetic peptide
CPYLC

SEQ ID NO: 236 - protein, 5 aa, Artificial Sequence
  synthetic peptide
CPYIC

SEQ ID NO: 237 - protein, 5 aa, Artificial Sequence
  synthetic peptide
CPYMC

SEQ ID NO: 238 - protein, 5 aa, Artificial Sequence
  synthetic peptide
CPYFC

SEQ ID NO: 239 - protein, 5 aa, Artificial Sequence
  synthetic peptide
CPYWC

SEQ ID NO: 240 - protein, 5 aa, Artificial Sequence
  synthetic peptide
CPYPC

SEQ ID NO: 241 - protein, 5 aa, Artificial Sequence
  synthetic peptide
CPYSC

SEQ ID NO: 242 - protein, 5 aa, Artificial Sequence
  synthetic peptide
CPYTC

SEQ ID NO: 243 - protein, 5 aa, Artificial Sequence
  synthetic peptide
CPYCC

SEQ ID NO: 244 - protein, 5 aa, Artificial Sequence
  synthetic peptide
CPYYC

SEQ ID NO: 245 - protein, 5 aa, Artificial Sequence
  synthetic peptide
CPYNC

SEQ ID NO: 246 - protein, 5 aa, Artificial Sequence
  synthetic peptide
CPYQC

SEQ ID NO: 247 - protein, 5 aa, Artificial Sequence
  synthetic peptide
CPYDC

SEQ ID NO: 248 - protein, 5 aa, Artificial Sequence
  synthetic peptide
CPYEC

SEQ ID NO: 249 - protein, 5 aa, Artificial Sequence
  synthetic peptide
CPYLC

SEQ ID NO: 250 - protein, 5 aa, Artificial Sequence
  synthetic peptide
  misc_feature (2)..(2): Xaa can be any naturally occurring amino acid
CXHGC

SEQ ID NO: 251 - protein, 5 aa, Artificial Sequence
  synthetic peptide
CKHGC

SEQ ID NO: 252 - protein, 5 aa, Artificial Sequence
  synthetic peptide
CRHGC

SEQ ID NO: 253 - protein, 5 aa, Artificial Sequence
  synthetic peptide
CHHGC

SEQ ID NO: 254 - protein, 5 aa, Artificial Sequence
  synthetic peptide
CGHGC

SEQ ID NO: 255 - protein, 5 aa, Artificial Sequence
  synthetic peptide
CAHGC

SEQ ID NO: 256 - protein, 5 aa, Artificial Sequence
  synthetic peptide
CVHGC

SEQ ID NO: 257 - protein, 5 aa, Artificial Sequence
  synthetic peptide
CLHGC

SEQ ID NO: 258 - protein, 5 aa, Artificial Sequence
  synthetic peptide
CIHGC

SEQ ID NO: 259 - protein, 5 aa, Artificial Sequence
  synthetic peptide
CMHGC

SEQ ID NO: 260 - protein, 5 aa, Artificial Sequence
  synthetic peptide
CFHGC

SEQ ID NO: 261 - protein, 5 aa, Artificial Sequence
  synthetic peptide
CWHGC

SEQ ID NO: 262 - protein, 5 aa, Artificial Sequence
  synthetic peptide
CPHGC

SEQ ID NO: 263 - protein, 5 aa, Artificial Sequence
  synthetic peptide
CSHGC

SEQ ID NO: 264 - protein, 5 aa, Artificial Sequence
  synthetic peptide
CTHGC

SEQ ID NO: 265 - protein, 5 aa, Artificial Sequence
  synthetic peptide
CCHGC

SEQ ID NO: 266 - protein, 5 aa, Artificial Sequence
  synthetic peptide
CYHGC

SEQ ID NO: 267 - protein, 5 aa, Artificial Sequence
  synthetic peptide
CNHGC

SEQ ID NO: 268 - protein, 5 aa, Artificial Sequence
  synthetic peptide
CQHGC

SEQ ID NO: 269 - protein, 5 aa, Artificial Sequence
  synthetic peptide
CDHGC

SEQ ID NO: 270 - protein, 5 aa, Artificial Sequence
  synthetic peptide
CEHGC

SEQ ID NO: 271 - protein, 5 aa, Artificial Sequence
  synthetic peptide
CKHGC

SEQ ID NO: 272 - protein, 5 aa, Artificial Sequence
  synthetic peptide
  misc_feature (3)..(3): Xaa can be any naturally occurring amino acid
CGXHC

SEQ ID NO: 273 - protein, 5 aa, Artificial Sequence
  synthetic peptide
CGKHC

SEQ ID NO: 274 - protein, 5 aa, Artificial Sequence
  synthetic peptide
CGRHC

SEQ ID NO: 275 - protein, 5 aa, Artificial Sequence
  synthetic peptide
CGHHC

SEQ ID NO: 276 - skipped

SEQ ID NO: 277 - protein, 5 aa, Artificial Sequence
  synthetic peptide
CGAHC

SEQ ID NO: 278 - protein, 5 aa, Artificial Sequence
  synthetic peptide
CGVHC

SEQ ID NO: 279 - protein, 5 aa, Artificial Sequence
  synthetic peptide
CGLHC

SEQ ID NO: 280 - protein, 5 aa, Artificial Sequence
  synthetic peptide
CGIHC

SEQ ID NO: 281 - protein, 5 aa, Artificial Sequence
  synthetic peptide
CGMHC

SEQ ID NO: 282 - protein, 5 aa, Artificial Sequence
  synthetic peptide
CGFHC

SEQ ID NO: 283 - protein, 5 aa, Artificial Sequence
  synthetic peptide
CGWHC

SEQ ID NO: 284 - protein, 5 aa, Artificial Sequence
  synthetic peptide
CGPHC

SEQ ID NO: 285 - protein, 5 aa, Artificial Sequence
  synthetic peptide
CGSHC

SEQ ID NO: 286 - protein, 5 aa, Artificial Sequence
  synthetic peptide
CGTHC

SEQ ID NO: 287 - protein, 5 aa, Artificial Sequence
  synthetic peptide
CGCHC

SEQ ID NO: 288 - protein, 5 aa, Artificial Sequence
  synthetic peptide
CGYHC

SEQ ID NO: 289 - protein, 5 aa, Artificial Sequence
  synthetic peptide
CGNHC

SEQ ID NO: 290 - protein, 5 aa, Artificial Sequence
  synthetic peptide
CGQHC

SEQ ID NO: 291 - protein, 5 aa, Artificial Sequence
  synthetic peptide
CGDHC

SEQ ID NO: 292 - protein, 5 aa, Artificial Sequence
  synthetic peptide
CGEHC

SEQ ID NO: 293 - protein, 5 aa, Artificial Sequence
  synthetic peptide
CGLHC

SEQ ID NO: 294 - protein, 5 aa, Artificial Sequence
  synthetic peptide
  misc_feature (4)..(4): Xaa can be any naturally occurring amino acid
CHGXC

SEQ ID NO: 295 - protein, 5 aa, Artificial Sequence
  synthetic peptide
CHGKC

SEQ ID NO: 296 - protein, 5 aa, Artificial Sequence
  synthetic peptide
CHGRC

SEQ ID NO: 297 - protein, 5 aa, Artificial Sequence
  synthetic peptide
CHGHC

SEQ ID NO: 298 - protein, 5 aa, Artificial Sequence
  synthetic peptide
CHGGC

SEQ ID NO: 299 - protein, 5 aa, Artificial Sequence
  synthetic peptide
CHGAC

SEQ ID NO: 300 - protein, 5 aa, Artificial Sequence
  synthetic peptide
CHGVC

SEQ ID NO: 301 - protein, 5 aa, Artificial Sequence
  synthetic peptide
CHGLC

SEQ ID NO: 302 - protein, 5 aa, Artificial Sequence
  synthetic peptide
CHGIC

SEQ ID NO: 303 - protein, 5 aa, Artificial Sequence
  synthetic peptide
CHGMC

SEQ ID NO: 304 - protein, 5 aa, Artificial Sequence
  synthetic peptide
CHGFC

SEQ ID NO: 305 - protein, 5 aa, Artificial Sequence
  synthetic peptide
CHGWC

SEQ ID NO: 306 - protein, 5 aa, Artificial Sequence
  synthetic peptide
CHGPC

SEQ ID NO: 307 - protein, 5 aa, Artificial Sequence
  synthetic peptide
CHGSC

SEQ ID NO: 308 - protein, 5 aa, Artificial Sequence
  synthetic peptide
CHGTC

SEQ ID NO: 309 - protein, 5 aa, Artificial Sequence
  synthetic peptide
CHGCC

SEQ ID NO: 310 - protein, 5 aa, Artificial Sequence
  synthetic peptide
CHGYC

SEQ ID NO: 311 - protein, 5 aa, Artificial Sequence
  synthetic peptide
CHGNC

SEQ ID NO: 312 - protein, 5 aa, Artificial Sequence
  synthetic peptide
CHGQC

SEQ ID NO: 313 - protein, 5 aa, Artificial Sequence
  synthetic peptide
CHGDC

SEQ ID NO: 314 - protein, 5 aa, Artificial Sequence
  synthetic peptide
CHGEC

SEQ ID NO: 315 - protein, 5 aa, Artificial Sequence
  synthetic peptide
CHGLC

SEQ ID NO: 316 - protein, 5 aa, Artificial Sequence
  synthetic peptide
  misc_feature (2)..(2): Xaa can be any naturally occurring amino acid
CXGPC

SEQ ID NO: 317 - protein, 5 aa, Artificial Sequence
  synthetic peptide
CKGPC

SEQ ID NO: 318 - protein, 5 aa, Artificial Sequence
  synthetic peptide
CRGPC

SEQ ID NO: 319 - protein, 5 aa, Artificial Sequence
  synthetic peptide
CHGPC

SEQ ID NO: 320 - protein, 5 aa, Artificial Sequence
  synthetic peptide
CGGPC

SEQ ID NO: 321 - protein, 5 aa, Artificial Sequence
  synthetic peptide
CAGPC

SEQ ID NO: 322 - protein, 5 aa, Artificial Sequence
  synthetic peptide
CVGPC

SEQ ID NO: 323 - protein, 5 aa, Artificial Sequence
  synthetic peptide
CLGPC

SEQ ID NO: 324 - protein, 5 aa, Artificial Sequence
  synthetic peptide
CIGPC

SEQ ID NO: 325 - protein, 5 aa, Artificial Sequence
  synthetic peptide
CMGPC

SEQ ID NO: 326 - protein, 5 aa, Artificial Sequence
  synthetic peptide
CFGPC

SEQ ID NO: 327 - protein, 5 aa, Artificial Sequence
  synthetic peptide
CWGPC

SEQ ID NO: 328 - protein, 5 aa, Artificial Sequence
  synthetic peptide
CPGPC

SEQ ID NO: 329 - protein, 5 aa, Artificial Sequence
  synthetic peptide
CSGPC

SEQ ID NO: 330 - protein, 5 aa, Artificial Sequence
  synthetic peptide
CTGPC

SEQ ID NO: 331 - protein, 5 aa, Artificial Sequence
  synthetic peptide
CCGPC

SEQ ID NO: 332 - protein, 5 aa, Artificial Sequence
  synthetic peptide
CYGPC

SEQ ID NO: 333 - protein, 5 aa, Artificial Sequence
  synthetic peptide
CNGPC

SEQ ID NO: 334 - protein, 5 aa, Artificial Sequence
  synthetic peptide
CQGPC

SEQ ID NO: 335 - protein, 5 aa, Artificial Sequence
  synthetic peptide
CDGPC

SEQ ID NO: 336 - protein, 5 aa, Artificial Sequence
  synthetic peptide
CEGPC

SEQ ID NO: 337 - protein, 5 aa, Artificial Sequence
  synthetic peptide
CKGPC

SEQ ID NO: 338 - protein, 5 aa, Artificial Sequence
  synthetic peptide
  misc_feature (3)..(3): Xaa can be any naturally occurring amino acid
CGXPC

SEQ ID NO: 339 - protein, 5 aa, Artificial Sequence
  synthetic peptide
CGKPC

SEQ ID NO: 340 - protein, 5 aa, Artificial Sequence
  synthetic peptide
CGRPC

SEQ ID NO: 341 - protein, 5 aa, Artificial Sequence
  synthetic peptide
CGHPC

SEQ ID NO: 342 - protein, 5 aa, Artificial Sequence
  synthetic peptide
CGGPC

SEQ ID NO: 343 - protein, 5 aa, Artificial Sequence
  synthetic peptide
CGAPC

SEQ ID NO: 344 - protein, 5 aa, Artificial Sequence
  synthetic peptide
CGVPC

SEQ ID NO: 345 - protein, 5 aa, Artificial Sequence
  synthetic peptide
CGLPC

SEQ ID NO: 346 - protein, 5 aa, Artificial Sequence
  synthetic peptide
CGIPC

SEQ ID NO: 347 - protein, 5 aa, Artificial Sequence
  synthetic peptide
CGMPC

SEQ ID NO: 348 - protein, 5 aa, Artificial Sequence
  synthetic peptide
CGFPC

SEQ ID NO: 349 - protein, 5 aa, Artificial Sequence
  synthetic peptide
CGWPC

SEQ ID NO: 350 - protein, 5 aa, Artificial Sequence
  synthetic peptide
CGPPC

SEQ ID NO: 351 - protein, 5 aa, Artificial Sequence
  synthetic peptide
CGSPC

SEQ ID NO: 352 - protein, 5 aa, Artificial Sequence
  synthetic peptide
CGTPC

SEQ ID NO: 353 - protein, 5 aa, Artificial Sequence
  synthetic peptide
CGCPC

SEQ ID NO: 354 - protein, 5 aa, Artificial Sequence
  synthetic peptide
CGYPC

SEQ ID NO: 355 - protein, 5 aa, Artificial Sequence
  synthetic peptide
CGNPC

SEQ ID NO: 356 - protein, 5 aa, Artificial Sequence
  synthetic peptide
CGQPC

SEQ ID NO: 357 - protein, 5 aa, Artificial Sequence
  synthetic peptide
CGDPC

SEQ ID NO: 358 - protein, 5 aa, Artificial Sequence
  synthetic peptide
CGEPC

SEQ ID NO: 359 - protein, 5 aa, Artificial Sequence
  synthetic peptide
CGLPC

SEQ ID NO: 360 - protein, 5 aa, Artificial Sequence
  synthetic peptide
  misc_feature (4)..(4): Xaa can be any naturally occurring amino acid
CGPXC

SEQ ID NO: 361 - protein, 5 aa, Artificial Sequence
  synthetic peptide
CGPKC

SEQ ID NO: 362 - protein, 5 aa, Artificial Sequence
  synthetic peptide
CGPRC

SEQ ID NO: 363 - protein, 5 aa, Artificial Sequence
  synthetic peptide
CGPHC

SEQ ID NO: 364 - protein, 5 aa, Artificial Sequence
  synthetic peptide
CGPGC

SEQ ID NO: 365 - protein, 5 aa, Artificial Sequence
  synthetic peptide
CGPAC

SEQ ID NO: 366 - protein, 5 aa, Artificial Sequence
  synthetic peptide
CGPVC

SEQ ID NO: 367 - protein, 5 aa, Artificial Sequence
  synthetic peptide
CGPLC

SEQ ID NO: 368 - protein, 5 aa, Artificial Sequence
  synthetic peptide
CGPIC

SEQ ID NO: 369 - protein, 5 aa, Artificial Sequence
  synthetic peptide
CGPMC

SEQ ID NO: 370 - protein, 5 aa, Artificial Sequence
  synthetic peptide
CGPFC

SEQ ID NO: 371 - protein, 5 aa, Artificial Sequence
  synthetic peptide
CGPWC

SEQ ID NO: 372 - protein, 5 aa, Artificial Sequence
  synthetic peptide
CGPPC

SEQ ID NO: 373 - protein, 5 aa, Artificial Sequence
  synthetic peptide
CGPSC

SEQ ID NO: 374 - protein, 5 aa, Artificial Sequence
  synthetic peptide
CGPTC

SEQ ID NO: 375 - protein, 5 aa, Artificial Sequence
  synthetic peptide
CGPCC

SEQ ID NO: 376 - protein, 5 aa, Artificial Sequence
  synthetic peptide
CGPYC

SEQ ID NO: 377 - protein, 5 aa, Artificial Sequence
  synthetic peptide
CGPNC

SEQ ID NO: 378 - protein, 5 aa, Artificial Sequence
  synthetic peptide
CGPQC

SEQ ID NO: 379 - protein, 5 aa, Artificial Sequence
  synthetic peptide
CGPDC

SEQ ID NO: 380 - protein, 5 aa, Artificial Sequence
  synthetic peptide
CGPEC

SEQ ID NO: 381 - protein, 5 aa, Artificial Sequence
  synthetic peptide
CGPLC

SEQ ID NO: 382 - protein, 5 aa, Artificial Sequence
  synthetic peptide
  misc_feature (2)..(2): Xaa can be any naturally occurring amino acid
CXGHC

SEQ ID NO: 383 - protein, 5 aa, Artificial Sequence
  synthetic peptide
CKGHC

SEQ ID NO: 384 - protein, 5 aa, Artificial Sequence
  synthetic peptide
CRGHC

SEQ ID NO: 385 - protein, 5 aa, Artificial Sequence
  synthetic peptide
CHGHC

SEQ ID NO: 386 - protein, 5 aa, Artificial Sequence
  synthetic peptide
CGGHC

SEQ ID NO: 387 - protein, 5 aa, Artificial Sequence
  synthetic peptide
CAGHC

SEQ ID NO: 388 - protein, 5 aa, Artificial Sequence
  synthetic peptide
CVGHC

SEQ ID NO: 389 - protein, 5 aa, Artificial Sequence
  synthetic peptide
CLGHC

SEQ ID NO: 390 - protein, 5 aa, Artificial Sequence
  synthetic peptide
CIGHC

SEQ ID NO: 391 - protein, 5 aa, Artificial Sequence
  synthetic peptide
CMGHC

SEQ ID NO: 392 - protein, 5 aa, Artificial Sequence
  synthetic peptide
CFGHC

SEQ ID NO: 393 - protein, 5 aa, Artificial Sequence
  synthetic peptide
CWGHC

SEQ ID NO: 394 - protein, 5 aa, Artificial Sequence
  synthetic peptide
CPGHC

SEQ ID NO: 395 - protein, 5 aa, Artificial Sequence
  synthetic peptide
CSGHC

SEQ ID NO: 396 - protein, 5 aa, Artificial Sequence
  synthetic peptide
CTGHC

SEQ ID NO: 397 - protein, 5 aa, Artificial Sequence
  synthetic peptide
CCGHC

SEQ ID NO: 398 - protein, 5 aa, Artificial Sequence
  synthetic peptide
CYGHC

SEQ ID NO: 399 - protein, 5 aa, Artificial Sequence
  synthetic peptide
CNGHC

SEQ ID NO: 400 - protein, 5 aa, Artificial Sequence
  synthetic peptide
CQGHC

SEQ ID NO: 401 - protein, 5 aa, Artificial Sequence
  synthetic peptide
CDGHC

SEQ ID NO: 402 - protein, 5 aa, Artificial Sequence
  synthetic peptide
CEGHC

SEQ ID NO: 403 - protein, 5 aa, Artificial Sequence
  synthetic peptide
CKGHC

SEQ ID NO: 404 - protein, 5 aa, Artificial Sequence
  synthetic peptide
  misc_feature (3)..(3): Xaa can be any naturally occurring amino acid
CGXFC

SEQ ID NO: 405 - protein, 5 aa, Artificial Sequence
  synthetic peptide
CGKFC

SEQ ID NO: 406 - protein, 5 aa, Artificial Sequence
  synthetic peptide
CGRFC

SEQ ID NO: 407 - protein, 5 aa, Artificial Sequence
  synthetic peptide
CGHFC

SEQ ID NO: 408 - protein, 5 aa, Artificial Sequence
  synthetic peptide
CGGFC

SEQ ID NO: 409 - protein, 5 aa, Artificial Sequence
  synthetic peptide
CGAFC

SEQ ID NO: 410 - protein, 5 aa, Artificial Sequence
  synthetic peptide
CGVFC

SEQ ID NO: 411 - protein, 5 aa, Artificial Sequence
  synthetic peptide
CGLFC

SEQ ID NO: 412 - protein, 5 aa, Artificial Sequence
  synthetic peptide
CGIFC

SEQ ID NO: 413 - protein, 5 aa, Artificial Sequence
  synthetic peptide
CGMFC

SEQ ID NO: 414 - protein, 5 aa, Artificial Sequence
  synthetic peptide
CGFFC

SEQ ID NO: 415 - protein, 5 aa, Artificial Sequence
  synthetic peptide
CGWFC

SEQ ID NO: 416 - protein, 5 aa, Artificial Sequence
  synthetic peptide
CGPFC

SEQ ID NO: 417 - protein, 5 aa, Artificial Sequence
  synthetic peptide
CGSFC

SEQ ID NO: 418 - protein, 5 aa, Artificial Sequence
  synthetic peptide
CGTFC

SEQ ID NO: 419 - protein, 5 aa, Artificial Sequence
  synthetic peptide
CGCFC

SEQ ID NO: 420 - protein, 5 aa, Artificial Sequence
  synthetic peptide
CGYFC

SEQ ID NO: 421 - protein, 5 aa, Artificial Sequence
  synthetic peptide
CGNFC

SEQ ID NO: 422 - protein, 5 aa, Artificial Sequence
  synthetic peptide
CGQFC

SEQ ID NO: 423 - protein, 5 aa, Artificial Sequence
  synthetic peptide
CGDFC

SEQ ID NO: 424 - protein, 5 aa, Artificial Sequence
  synthetic peptide
CGEFC

SEQ ID NO: 425 - protein, 5 aa, Artificial Sequence
  synthetic peptide
CGLFC

SEQ ID NO: 426 - protein, 5 aa, Artificial Sequence
  synthetic peptide
  misc_feature (4)..(4): Xaa can be any naturally occurring amino acid
CGHXC

SEQ ID NO: 427 - protein, 5 aa, Artificial Sequence
  synthetic peptide
CGHKC

SEQ ID NO: 428 - protein, 5 aa, Artificial Sequence
  synthetic peptide
CGHRC

SEQ ID NO: 429 - protein, 5 aa, Artificial Sequence
  synthetic peptide
CGHHC

SEQ ID NO: 430 - protein, 5 aa, Artificial Sequence
  synthetic peptide
CGHGC

SEQ ID NO: 431 - protein, 5 aa, Artificial Sequence
  synthetic peptide
CGHAC

SEQ ID NO: 432 - protein, 5 aa, Artificial Sequence
  synthetic peptide
CGHVC

SEQ ID NO: 433 - protein, 5 aa, Artificial Sequence
  synthetic peptide
CGHLC

SEQ ID NO: 434 - protein, 5 aa, Artificial Sequence
  synthetic peptide
CGHIC

SEQ ID NO: 435 - protein, 5 aa, Artificial Sequence
  synthetic peptide
CGHMC

SEQ ID NO: 436 - protein, 5 aa, Artificial Sequence
  synthetic peptide
CGHFC

SEQ ID NO: 437 - protein, 5 aa, Artificial Sequence
  synthetic peptide
CGHWC

SEQ ID NO: 438 - protein, 5 aa, Artificial Sequence
  synthetic peptide
CGHPC

SEQ ID NO: 439 - protein, 5 aa, Artificial Sequence
  synthetic peptide
CGHSC

SEQ ID NO: 440 - protein, 5 aa, Artificial Sequence
  synthetic peptide
CGHTC

SEQ ID NO: 441 - protein, 5 aa, Artificial Sequence
  synthetic peptide
CGHCC

SEQ ID NO: 442 - protein, 5 aa, Artificial Sequence
  synthetic peptide
CGHYC

SEQ ID NO: 443 - protein, 5 aa, Artificial Sequence
  synthetic peptide
CGHNC

SEQ ID NO: 444 - protein, 5 aa, Artificial Sequence
  synthetic peptide
CGHQC

SEQ ID NO: 445 - protein, 5 aa, Artificial Sequence
  synthetic peptide
CGHDC

SEQ ID NO: 446 - protein, 5 aa, Artificial Sequence
  synthetic peptide
CGHEC

SEQ ID NO: 447 - protein, 5 aa, Artificial Sequence
  synthetic peptide
CGHLC

SEQ ID NO: 448 - protein, 5 aa, Artificial Sequence
  synthetic peptide
  misc_feature (2)..(2): Xaa can be any naturally occurring amino acid
CXGFC

SEQ ID NO: 449 - protein, 5 aa, Artificial Sequence
  synthetic peptide
CKGFC

SEQ ID NO: 450 - protein, 5 aa, Artificial Sequence
  synthetic peptide
CRGFC

SEQ ID NO: 451 - protein, 5 aa, Artificial Sequence
  synthetic peptide
CHGFC

SEQ ID NO: 452 - protein, 5 aa, Artificial Sequence
  synthetic peptide
CGGFC

SEQ ID NO: 453 - protein, 5 aa, Artificial Sequence
  synthetic peptide
CAGFC

SEQ ID NO: 454 - protein, 5 aa, Artificial Sequence
  synthetic peptide
CVGFC

SEQ ID NO: 455 - protein, 5 aa, Artificial Sequence
  synthetic peptide
CLGFC

SEQ ID NO: 456 - protein, 5 aa, Artificial Sequence
  synthetic peptide
CIGFC

SEQ ID NO: 457 - protein, 5 aa, Artificial Sequence
  synthetic peptide
CMGFC

SEQ ID NO: 458 - protein, 5 aa, Artificial Sequence
  synthetic peptide
CFGFC

SEQ ID NO: 459 - protein, 5 aa, Artificial Sequence
  synthetic peptide
CWGFC

SEQ ID NO: 460 - protein, 5 aa, Artificial Sequence
  synthetic peptide
CPGFC

SEQ ID NO: 461 - protein, 5 aa, Artificial Sequence
  synthetic peptide
CSGFC

SEQ ID NO: 462 - protein, 5 aa, Artificial Sequence
  synthetic peptide
CTGFC

SEQ ID NO: 463 - protein, 5 aa, Artificial Sequence
  synthetic peptide
CCGFC

SEQ ID NO: 464 - protein, 5 aa, Artificial Sequence
  synthetic peptide
CYGFC

SEQ ID NO: 465 - protein, 5 aa, Artificial Sequence
  synthetic peptide
CNGFC

SEQ ID NO: 466 - protein, 5 aa, Artificial Sequence
  synthetic peptide
CQGFC

SEQ ID NO: 467 - protein, 5 aa, Artificial Sequence
  synthetic peptide
CDGFC

SEQ ID NO: 468 - protein, 5 aa, Artificial Sequence
  synthetic peptide
CEGFC

SEQ ID NO: 469 - protein, 5 aa, Artificial Sequence
  synthetic peptide
CKGFC

SEQ ID NO: 470 - protein, 5 aa, Artificial Sequence
  synthetic peptide
  misc_feature (3)..(3): Xaa can be any naturally occurring amino acid
CGXFC

SEQ ID NO: 471 - protein, 5 aa, Artificial Sequence
  CGKFC
CGKFC

SEQ ID NO: 472 - protein, 5 aa, Artificial Sequence
  synthetic peptide
CGRFC

SEQ ID NO: 473 - protein, 5 aa, Artificial Sequence
  synthetic peptide
CGHFC

SEQ ID NO: 474 - protein, 5 aa, Artificial Sequence
  synthetic peptide
CGGFC

SEQ ID NO: 475 - protein, 5 aa, Artificial Sequence
  synthetic peptide
CGAFC

SEQ ID NO: 476 - protein, 5 aa, Artificial Sequence
  synthetic peptide
CGVFC

SEQ ID NO: 477 - protein, 5 aa, Artificial Sequence
  synthetic peptide
CGLFC

SEQ ID NO: 478 - protein, 5 aa, Artificial Sequence
  synthetic peptide
CGIFC

SEQ ID NO: 479 - protein, 5 aa, Artificial Sequence
  synthetic peptide
CGMFC

SEQ ID NO: 480 - protein, 5 aa, Artificial Sequence
  synthetic peptide
CGFFC

SEQ ID NO: 481 - protein, 5 aa, Artificial Sequence
  synthetic peptide
CGWFC

SEQ ID NO: 482 - protein, 5 aa, Artificial Sequence
  synthetic peptide
CGPFC

SEQ ID NO: 483 - protein, 5 aa, Artificial Sequence
  synthetic peptide
CGSFC

SEQ ID NO: 484 - protein, 5 aa, Artificial Sequence
  synthetic peptide
CGTFC

SEQ ID NO: 485 - protein, 5 aa, Artificial Sequence
  synthetic peptide
CGCFC

SEQ ID NO: 486 - protein, 5 aa, Artificial Sequence
  synthetic peptide
CGYFC

SEQ ID NO: 487 - protein, 5 aa, Artificial Sequence
  synthetic peptide
CGNFC

SEQ ID NO: 488 - protein, 5 aa, Artificial Sequence
  synthetic peptide
CGQFC

SEQ ID NO: 489 - protein, 5 aa, Artificial Sequence
  synthetic peptide
CGDFC

SEQ ID NO: 490 - protein, 5 aa, Artificial Sequence
  synthetic peptide
CGEFC

SEQ ID NO: 491 - protein, 5 aa, Artificial Sequence
  synthetic peptide
CGLFC

SEQ ID NO: 492 - protein, 5 aa, Artificial Sequence
  synthetic peptide
  misc_feature (4)..(4): Xaa can be any naturally occurring amino acid
CGFXC

SEQ ID NO: 493 - protein, 5 aa, Artificial Sequence
  synthetic peptide
CGFKC

SEQ ID NO: 494 - protein, 5 aa, Artificial Sequence
  synthetic peptide
CGFRC

SEQ ID NO: 495 - protein, 5 aa, Artificial Sequence
  synthetic peptide
CGFHC

SEQ ID NO: 496 - protein, 5 aa, Artificial Sequence
  synthetic peptide
CGFGC

SEQ ID NO: 497 - protein, 5 aa, Artificial Sequence
  synthetic peptide
CGFAC

SEQ ID NO: 498 - protein, 5 aa, Artificial Sequence
  synthetic peptide
CGFVC

SEQ ID NO: 499 - protein, 5 aa, Artificial Sequence
  synthetic peptide
CGFLC

SEQ ID NO: 500 - protein, 5 aa, Artificial Sequence
  synthetic peptide
CGFIC

SEQ ID NO: 501 - protein, 5 aa, Artificial Sequence
  synthetic peptide
CGFMC

SEQ ID NO: 502 - protein, 5 aa, Artificial Sequence
  synthetic peptide
CGFFC

SEQ ID NO: 503 - protein, 5 aa, Artificial Sequence
  synthetic peptide
CGFWC

SEQ ID NO: 504 - protein, 5 aa, Artificial Sequence
  synthetic peptide
CGFPC

SEQ ID NO: 505 - protein, 5 aa, Artificial Sequence
  synthetic peptide
CGFSC

SEQ ID NO: 506 - protein, 5 aa, Artificial Sequence
  synthetic peptide
CGFTC

SEQ ID NO: 507 - protein, 5 aa, Artificial Sequence
  synthetic peptide
CGFCC

SEQ ID NO: 508 - protein, 5 aa, Artificial Sequence
  synthetic peptide
CGFYC

SEQ ID NO: 509 - protein, 5 aa, Artificial Sequence
  synthetic peptide
CGFNC

SEQ ID NO: 510 - protein, 5 aa, Artificial Sequence
  synthetic peptide
CGFQC

SEQ ID NO: 511 - protein, 5 aa, Artificial Sequence
  synthetic peptide
CGFDC

SEQ ID NO: 512 - protein, 5 aa, Artificial Sequence
  synthetic peptide
CGFEC

SEQ ID NO: 513 - protein, 5 aa, Artificial Sequence
  synthetic peptide
CGFLC

SEQ ID NO: 514 - protein, 5 aa, Artificial Sequence
  synthetic peptide
  misc_feature (2)..(2): Xaa can be any naturally occurring amino acid
CXRLC

SEQ ID NO: 515 - protein, 5 aa, Artificial Sequence
  synthetic peptide
CKRLC

SEQ ID NO: 516 - protein, 5 aa, Artificial Sequence
  synthetic peptide
CRRLC

SEQ ID NO: 517 - protein, 5 aa, Artificial Sequence
  synthetic peptide
CHRLC

SEQ ID NO: 518 - protein, 5 aa, Artificial Sequence
  synthetic peptide
CGRLC

SEQ ID NO: 519 - protein, 5 aa, Artificial Sequence
  synthetic peptide
CARLC

SEQ ID NO: 520 - protein, 5 aa, Artificial Sequence
  synthetic peptide
CVRLC

SEQ ID NO: 521 - protein, 5 aa, Artificial Sequence
  synthetic peptide
CLRLC

SEQ ID NO: 522 - protein, 5 aa, Artificial Sequence
  synthetic peptide
CIRLC

SEQ ID NO: 523 - protein, 5 aa, Artificial Sequence
  synthetic peptide
CMRLC

SEQ ID NO: 524 - protein, 5 aa, Artificial Sequence
  synthetic peptide
CFRLC

SEQ ID NO: 525 - protein, 5 aa, Artificial Sequence
  synthetic peptide
CWRLC

SEQ ID NO: 526 - protein, 5 aa, Artificial Sequence
  synthetic peptide
CPRLC

SEQ ID NO: 527 - protein, 5 aa, Artificial Sequence
  synthetic peptide
CSRLC

SEQ ID NO: 528 - protein, 5 aa, Artificial Sequence
  synthetic peptide
CTRLC

SEQ ID NO: 529 - protein, 5 aa, Artificial Sequence
  synthetic peptide
CCRLC

SEQ ID NO: 530 - protein, 5 aa, Artificial Sequence
  synthetic peptide
CYRLC

SEQ ID NO: 531 - protein, 5 aa, Artificial Sequence
  synthetic peptide
CNRLC

SEQ ID NO: 532 - protein, 5 aa, Artificial Sequence
  synthetic peptide
CQRLC

SEQ ID NO: 533 - protein, 5 aa, Artificial Sequence
  synthetic peptide
CDRLC

SEQ ID NO: 534 - protein, 5 aa, Artificial Sequence
  synthetic peptide
CERLC

SEQ ID NO: 535 - protein, 5 aa, Artificial Sequence
  synthetic peptide
CKRLC

SEQ ID NO: 536 - protein, 5 aa, Artificial Sequence
  synthetic peptide
  misc_feature (3)..(3): Xaa can be any naturally occurring amino acid
CRXLC

SEQ ID NO: 537 - protein, 5 aa, Artificial Sequence
  synthetic peptide
CRKLC

SEQ ID NO: 538 - protein, 5 aa, Artificial Sequence
  synthetic peptide
CRRLC

SEQ ID NO: 539 - protein, 5 aa, Artificial Sequence
  synthetic peptide
CRHLC

SEQ ID NO: 540 - protein, 5 aa, Artificial Sequence
  synthetic peptide
CRGLC

SEQ ID NO: 541 - protein, 5 aa, Artificial Sequence
  synthetic peptide
CRALC

SEQ ID NO: 542 - protein, 5 aa, Artificial Sequence
  synthetic peptide
CRVLC

SEQ ID NO: 543 - protein, 5 aa, Artificial Sequence
  synthetic peptide
CRLLC

SEQ ID NO: 544 - protein, 5 aa, Artificial Sequence
  synthetic peptide
CRILC

SEQ ID NO: 545 - protein, 5 aa, Artificial Sequence
  synthetic peptide
CRMLC

SEQ ID NO: 546 - protein, 5 aa, Artificial Sequence
  synthetic peptide
CRFLC

SEQ ID NO: 547 - protein, 5 aa, Artificial Sequence
  synthetic peptide
CRWLC

SEQ ID NO: 548 - protein, 5 aa, Artificial Sequence
  synthetic peptide
CRPLC

SEQ ID NO: 549 - protein, 5 aa, Artificial Sequence
  synthetic peptide
CRSLC

SEQ ID NO: 550 - protein, 5 aa, Artificial Sequence
  synthetic peptide
CRTLC

SEQ ID NO: 551 - protein, 5 aa, Artificial Sequence
  synthetic peptide
CRCLC

SEQ ID NO: 552 - protein, 5 aa, Artificial Sequence
  synthetic peptide
CRYLC

SEQ ID NO: 553 - protein, 5 aa, Artificial Sequence
  synthetic peptide
CRNLC

SEQ ID NO: 554 - protein, 5 aa, Artificial Sequence
  synthetic peptide
CRQLC

SEQ ID NO: 555 - protein, 5 aa, Artificial Sequence
  synthetic peptide
CRDLC

SEQ ID NO: 556 - protein, 5 aa, Artificial Sequence
  synthetic peptide
CRELC

SEQ ID NO: 557 - protein, 5 aa, Artificial Sequence
  synthetic peptide
CRLLC

SEQ ID NO: 558 - protein, 5 aa, Artificial Sequence
  synthetic peptide
  misc_feature (4)..(4): Xaa can be any naturally occurring amino acid
CRLXC

SEQ ID NO: 559 - protein, 5 aa, Artificial Sequence
  synthetic peptide
CRLKC

SEQ ID NO: 560 - protein, 5 aa, Artificial Sequence
  synthetic peptide
CRLRC

SEQ ID NO: 561 - protein, 5 aa, Artificial Sequence
  synthetic peptide
CRLHC

SEQ ID NO: 562 - protein, 5 aa, Artificial Sequence
  synthetic peptide
CRLGC

SEQ ID NO: 563 - protein, 5 aa, Artificial Sequence
  synthetic peptide
CRLAC

SEQ ID NO: 564 - protein, 5 aa, Artificial Sequence
  synthetic peptide
CRLVC

SEQ ID NO: 565 - protein, 5 aa, Artificial Sequence
  synthetic peptide
CRLLC

SEQ ID NO: 566 - protein, 5 aa, Artificial Sequence
  synthetic peptide
CRLIC

SEQ ID NO: 567 - protein, 5 aa, Artificial Sequence
  synthetic peptide
CRLMC

SEQ ID NO: 568 - protein, 5 aa, Artificial Sequence
  synthetic peptide
CRLFC

SEQ ID NO: 569 - protein, 5 aa, Artificial Sequence
  synthetic peptide
CRLWC

SEQ ID NO: 570 - protein, 5 aa, Artificial Sequence
  synthetic peptide
CRLPC

SEQ ID NO: 571 - protein, 5 aa, Artificial Sequence
  synthetic peptide
CRLSC

SEQ ID NO: 572 - protein, 5 aa, Artificial Sequence
  synthetic peptide
CRLTC

SEQ ID NO: 573 - protein, 5 aa, Artificial Sequence
  synthetic peptide
CRLCC

SEQ ID NO: 574 - protein, 5 aa, Artificial Sequence
  synthetic peptide
CRLYC

SEQ ID NO: 575 - protein, 5 aa, Artificial Sequence
  synthetic peptide
CRLNC

SEQ ID NO: 576 - protein, 5 aa, Artificial Sequence
  synthetic peptide
CRLQC

SEQ ID NO: 577 - protein, 5 aa, Artificial Sequence
  synthetic peptide
CRLDC

SEQ ID NO: 578 - protein, 5 aa, Artificial Sequence
  synthetic peptide
CRLEC

SEQ ID NO: 579 - protein, 5 aa, Artificial Sequence
  synthetic peptide
CRLLC

SEQ ID NO: 580 - protein, 5 aa, Artificial Sequence
  synthetic peptide
  misc_feature (2)..(2): Xaa can be any naturally occurring amino acid
CXHPC

SEQ ID NO: 581 - protein, 5 aa, Artificial Sequence
  synthetic peptide
CKHPC

SEQ ID NO: 582 - protein, 5 aa, Artificial Sequence
  synthetic peptide
CRHPC

SEQ ID NO: 583 - protein, 5 aa, Artificial Sequence
  synthetic peptide
CHHPC

SEQ ID NO: 584 - protein, 5 aa, Artificial Sequence
  synthetic peptide
CGHPC

SEQ ID NO: 585 - protein, 5 aa, Artificial Sequence
  synthetic peptide
CAHPC

SEQ ID NO: 586 - protein, 5 aa, Artificial Sequence
  synthetic peptide
CVHPC

SEQ ID NO: 587 - protein, 5 aa, Artificial Sequence
  synthetic peptide
CLHPC

SEQ ID NO: 588 - protein, 5 aa, Artificial Sequence
  synthetic peptide
CIHPC

SEQ ID NO: 589 - protein, 5 aa, Artificial Sequence
  synthetic peptide
CMHPC

SEQ ID NO: 590 - protein, 5 aa, Artificial Sequence
  synthetic peptide
CFHPC

SEQ ID NO: 591 - protein, 5 aa, Artificial Sequence
  synthetic peptide
CWHPC

SEQ ID NO: 592 - protein, 5 aa, Artificial Sequence
  synthetic peptide
CPHPC

SEQ ID NO: 593 - protein, 5 aa, Artificial Sequence
  synthetic peptide
CSHPC

SEQ ID NO: 594 - protein, 5 aa, Artificial Sequence
  synthetic peptide
CTHPC

SEQ ID NO: 595 - protein, 5 aa, Artificial Sequence
  synthetic peptide
CCHPC

SEQ ID NO: 596 - protein, 5 aa, Artificial Sequence
  synthetic peptide
CYHPC

SEQ ID NO: 597 - protein, 5 aa, Artificial Sequence
  synthetic peptide
CNHPC

SEQ ID NO: 598 - protein, 5 aa, Artificial Sequence
  synthetic peptide
CQHPC

SEQ ID NO: 599 - protein, 5 aa, Artificial Sequence
  synthetic peptide
CDHPC

SEQ ID NO: 600 - protein, 5 aa, Artificial Sequence
  synthetic peptide
CEHPC

SEQ ID NO: 601 - protein, 5 aa, Artificial Sequence
  synthetic peptide
CKHPC

SEQ ID NO: 602 - protein, 5 aa, Artificial Sequence
  synthetic peptide
  misc_feature (3)..(3): Xaa can be any naturally occurring amino acid
CHXPC

SEQ ID NO: 603 - protein, 5 aa, Artificial Sequence
  synthetic peptide
CHKPC

SEQ ID NO: 604 - protein, 5 aa, Artificial Sequence
  synthetic peptide
CHRPC

SEQ ID NO: 605 - protein, 5 aa, Artificial Sequence
  synthetic peptide
CHHPC

SEQ ID NO: 606 - protein, 5 aa, Artificial Sequence
  synthetic peptide
CHGPC

SEQ ID NO: 607 - protein, 5 aa, Artificial Sequence
  synthetic peptide
CHAPC

SEQ ID NO: 608 - protein, 5 aa, Artificial Sequence
  synthetic peptide
CHVPC

SEQ ID NO: 609 - protein, 5 aa, Artificial Sequence
  synthetic peptide
CHLPC

SEQ ID NO: 610 - protein, 5 aa, Artificial Sequence
  synthetic peptide
CHIPC

SEQ ID NO: 611 - protein, 5 aa, Artificial Sequence
  synthetic peptide
CHMPC

SEQ ID NO: 612 - protein, 5 aa, Artificial Sequence
  synthetic peptide
CHFPC

SEQ ID NO: 613 - protein, 5 aa, Artificial Sequence
  synthetic peptide
CHWPC

SEQ ID NO: 614 - protein, 5 aa, Artificial Sequence
  synthetic peptide
CHPPC

SEQ ID NO: 615 - protein, 5 aa, Artificial Sequence
  synthetic peptide
CHSPC

SEQ ID NO: 616 - protein, 5 aa, Artificial Sequence
  synthetic peptide
CHTPC

SEQ ID NO: 617 - protein, 5 aa, Artificial Sequence
  synthetic peptide
CHCPC

SEQ ID NO: 618 - protein, 5 aa, Artificial Sequence
  synthetic peptide
CHYPC

SEQ ID NO: 619 - protein, 5 aa, Artificial Sequence
  synthetic peptide
CHNPC

SEQ ID NO: 620 - protein, 5 aa, Artificial Sequence
  synthetic peptide
CHQPC

SEQ ID NO: 621 - protein, 5 aa, Artificial Sequence
  synthetic peptide
CHDPC

SEQ ID NO: 622 - protein, 5 aa, Artificial Sequence
  synthetic peptide
CHEPC

SEQ ID NO: 623 - protein, 5 aa, Artificial Sequence
  synthetic peptide
CHLPC

SEQ ID NO: 624 - protein, 5 aa, Artificial Sequence
  synthetic peptide
  misc_feature (4)..(4): Xaa can be any naturally occurring amino acid
CHPXC

SEQ ID NO: 625 - protein, 5 aa, Artificial Sequence
  synthetic peptide
CHPKC

SEQ ID NO: 626 - protein, 5 aa, Artificial Sequence
  synthetic peptide
CHPRC

SEQ ID NO: 627 - protein, 5 aa, Artificial Sequence
  synthetic peptide
CHPHC

SEQ ID NO: 628 - protein, 5 aa, Artificial Sequence
  synthetic peptide
CHPGC

SEQ ID NO: 629 - protein, 5 aa, Artificial Sequence
  synthetic peptide
CHPAC

SEQ ID NO: 630 - protein, 5 aa, Artificial Sequence
  synthetic peptide
CHPVC

SEQ ID NO: 631 - protein, 5 aa, Artificial Sequence
  synthetic peptide
CHPLC

SEQ ID NO: 632 - protein, 5 aa, Artificial Sequence
  synthetic peptide
CHPIC

SEQ ID NO: 633 - protein, 5 aa, Artificial Sequence
  synthetic peptide
CHPMC

SEQ ID NO: 634 - protein, 5 aa, Artificial Sequence
  synthetic peptide
CHPFC

SEQ ID NO: 635 - protein, 5 aa, Artificial Sequence
  synthetic peptide
CHPWC

SEQ ID NO: 636 - protein, 5 aa, Artificial Sequence
  synthetic peptide
CHPPC

SEQ ID NO: 637 - protein, 5 aa, Artificial Sequence
  synthetic peptide
CHPSC

SEQ ID NO: 638 - protein, 5 aa, Artificial Sequence
  synthetic peptide
CHPTC

SEQ ID NO: 639 - protein, 5 aa, Artificial Sequence
  synthetic peptide
CHPCC

SEQ ID NO: 640 - protein, 5 aa, Artificial Sequence
  synthetic peptide
CHPYC

SEQ ID NO: 641 - protein, 5 aa, Artificial Sequence
  synthetic peptide
CHPNC

SEQ ID NO: 642 - protein, 5 aa, Artificial Sequence
  synthetic peptide
CHPQC

SEQ ID NO: 643 - protein, 5 aa, Artificial Sequence
  synthetic peptide
CHPDC

SEQ ID NO: 644 - protein, 5 aa, Artificial Sequence
  synthetic peptide
CHPEC

SEQ ID NO: 645 - protein, 5 aa, Artificial Sequence
  synthetic peptide
CHPLC

SEQ ID NO: 646 - protein, 6 aa, Artificial Sequence
  synthetic peptide
  PEPTIDE (2)..(5): G, A, V, L, I, M, F, W, P, S, T, C, Y, N, Q, D, E, K, R, and H, or non-natural basic amino acid
CXXXXC

SEQ ID NO: 647 - protein, 6 aa, Artificial Sequence
  synthetic peptide
CLAVLC

SEQ ID NO: 648 - protein, 6 aa, Artificial Sequence
  synthetic peptide
CTVQAC

SEQ ID NO: 649 - protein, 6 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
CXAVLC

SEQ ID NO: 650 - protein, 6 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
CLXVLC

SEQ ID NO: 651 - protein, 6 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
CLAXLC

SEQ ID NO: 652 - protein, 6 aa, Artificial Sequence
  synthetic peptide
  PEPTIDE (5)..(5): G, A, V, L, I, M, F, W, P, S, T, C, Y, N, Q, D, E, K, R, and H, or non-natural basic amino acid
CLAVXC

SEQ ID NO: 653 - protein, 6 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
CXVQAC

SEQ ID NO: 654 - protein, 6 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
CTXQAC

SEQ ID NO: 655 - protein, 6 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
CTVXAC

SEQ ID NO: 656 - protein, 6 aa, Artificial Sequence
  synthetic peptide
  PEPTIDE (5)..(5): G, A, V, L, I, M, F, W, P, S, T, C, Y, N, Q, D, E, K, R, and H, or non-natural basic amino acid
CTVQXC

SEQ ID NO: 657 - protein, 6 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
CXAVHC

SEQ ID NO: 658 - protein, 6 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
CGXVHC

SEQ ID NO: 659 - protein, 6 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
CGAXHC

SEQ ID NO: 660 - protein, 6 aa, Artificial Sequence
  synthetic peptide
  PEPTIDE (5)..(5): G, A, V, L, I, M, F, W, P, S, T, C, Y, N, Q, D, E, K, R, and H, or non-natural basic amino acid
CGAVXC

SEQ ID NO: 661 - protein, 7 aa, Artificial Sequence
  synthetic peptide
  PEPTIDE (2)..(6): G, A, V, L, I, M, F, W, P, S, T, C, Y, N, Q, D, E, K, R, and H, or non-natural basic amino acid
CXXXXXC

SEQ ID NO: 662 - protein, 7 aa, Artificial Sequence
  synthetic peptide
CPAFPLC

SEQ ID NO: 663 - protein, 7 aa, Artificial Sequence
  synthetic peptide
CDQGGEC

SEQ ID NO: 664 - protein, 7 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
CXAFPLC

SEQ ID NO: 665 - protein, 7 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
CPXFPLC

SEQ ID NO: 666 - protein, 7 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
CPAXPLC

SEQ ID NO: 667 - protein, 7 aa, Artificial Sequence
  synthetic peptide
  PEPTIDE (5)..(5): G, A, V, L, I, M, F, W, P, S, T, C, Y, N, Q, D, E, K, R, and H, or non-natural basic amino acid
CPAFXLC

SEQ ID NO: 668 - protein, 7 aa, Artificial Sequence
  synthetic peptide
  misc_feature (6)..(6): Xaa can be any naturally occurring amino acid
  PEPTIDE (7)..(7): G, A, V, L, I, M, F, W, P, S, T, C, Y, N, Q, D, E, K, R, and H, or non-natural basic amino acid
CPAFPXC

SEQ ID NO: 669 - protein, 7 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
CXQGGEC

SEQ ID NO: 670 - protein, 7 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
CDXGGEC

SEQ ID NO: 671 - protein, 7 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
CDQXGEC

SEQ ID NO: 672 - protein, 7 aa, Artificial Sequence
  synthetic peptide
  PEPTIDE (5)..(5): G, A, V, L, I, M, F, W, P, S, T, C, Y, N, Q, D, E, K, R, and H, or non-natural basic amino acid
CDQGXEC

SEQ ID NO: 673 - protein, 7 aa, Artificial Sequence
  synthetic peptide
  PEPTIDE (6)..(6): G, A, V, L, I, M, F, W, P, S, T, C, Y, N, Q, D, E, K, R, and H, or non-natural basic amino acid
CDQGGXC

SEQ ID NO: 674 - protein, 8 aa, Artificial Sequence
  synthetic peptide
  PEPTIDE (2)..(7): G, A, V, L, I, M, F, W, P, S, T, C, Y, N, Q, D, E, K, R, and H, or non-natural basic amino acid
CXXXXXXC

SEQ ID NO: 675 - protein, 8 aa, Artificial Sequence
  synthetic peptide
CDIADKYC

SEQ ID NO: 676 - protein, 8 aa, Artificial Sequence
  synthetic peptide
  PEPTIDE (2)..(2): G, A, V, L, I, M, F, W, P, S, T, C, Y, N, Q, D, E, K, R, and H, or non-natural basic amino acid
CXIADKYC

SEQ ID NO: 677 - protein, 8 aa, Artificial Sequence
  synthetic peptide
  PEPTIDE (3)..(3): G, A, V, L, I, M, F, W, P, S, T, C, Y, N, Q, D, E, K, R, and H, or non-natural basic amino acid
CDXADKYC

SEQ ID NO: 678 - protein, 8 aa, Artificial Sequence
  synthetic peptide
  PEPTIDE (4)..(4): G, A, V, L, I, M, F, W, P, S, T, C, Y, N, Q, D, E, K, R, and H, or non-natural basic amino acid
CDIXDKYC

SEQ ID NO: 679 - protein, 8 aa, Artificial Sequence
  synthetic peptide
  PEPTIDE (5)..(5): G, A, V, L, I, M, F, W, P, S, T, C, Y, N, Q, D, E, K, R, and H, or non-natural basic amino acid
CDIAXKYC

SEQ ID NO: 680 - protein, 8 aa, Artificial Sequence
  synthetic peptide
  PEPTIDE (6)..(6): G, A, V, L, I, M, F, W, P, S, T, C, Y, N, Q, D, E, K, R, and H, or non-natural basic amino acid
CDIADXYC

SEQ ID NO: 681 - protein, 8 aa, Artificial Sequence
  synthetic peptide
  PEPTIDE (7)..(7): G, A, V, L, I, M, F, W, P, S, T, C, Y, N, Q, D, E, K, R, and H, or non-natural basic amino acid
CDIADKXC

SEQ ID NO: 682 - protein, 6 aa, Artificial Sequence
  synthetic peptide
CGAVHC

SEQ ID NO: 683 - protein, 3 aa, Artificial Sequence
  synthetic peptide
  PEPTIDE (2)..(2): K, H, or R
CXC

SEQ ID NO: 684 - protein, 5 aa, Artificial Sequence
  synthetic peptide
  misc_feature (2)..(2): Xaa can be any naturally occurring amino acid
  PEPTIDE (3)..(3): K, H, or R
  misc_feature (4)..(4): Xaa can be any naturally occurring amino acid
CXXXC

SEQ ID NO: 685 - protein, 5 aa, Artificial Sequence
  synthetic peptide
  misc_feature (2)..(3): Xaa can be any naturally occurring amino acid
  PEPTIDE (4)..(4): K, H, or R
CXXXC

SEQ ID NO: 686 - protein, 5 aa, Artificial Sequence
  synthetic peptide
  PEPTIDE (2)..(2): K, H, or R
  misc_feature (3)..(4): Xaa can be any naturally occurring amino acid
CXXXC

SEQ ID NO: 687 - protein, 3 aa, Artificial Sequence
  synthetic peptide
  PEPTIDE (1)..(1): K, H, or R
XCC

SEQ ID NO: 688 - protein, 4 aa, Artificial Sequence
  synthetic peptide
  PEPTIDE (1)..(1): K, H, or R
  misc_feature (3)..(3): Xaa can be any naturally occurring amino acid
XCXC

SEQ ID NO: 689 - protein, 5 aa, Artificial Sequence
  synthetic peptide
  PEPTIDE (1)..(1): K, H, or R
  misc_feature (2)..(4): Xaa can be any naturally occurring amino acid
XXXXC

SEQ ID NO: 690 - protein, 3 aa, Artificial Sequence
  synthetic peptide
  PEPTIDE (3)..(3): K, H, or R
CCX

SEQ ID NO: 691 - protein, 4 aa, Artificial Sequence
  synthetic peptide
  misc_feature (2)..(2): Xaa can be any naturally occurring amino acid
  PEPTIDE (4)..(4): K, H, or R
CXCX

SEQ ID NO: 692 - protein, 6 aa, Artificial Sequence
  synthetic peptide
  misc_feature (2)..(4): Xaa can be any naturally occurring amino acid
  PEPTIDE (6)..(6): K, H, or R
CXXXCX

SEQ ID NO: 693 - protein, 4 aa, Artificial Sequence
  synthetic peptide
  PEPTIDE (1)..(1): K, H, or R
  PEPTIDE (4)..(4): K, H, or R
XCCX

SEQ ID NO: 694 - protein, 5 aa, Artificial Sequence
  synthetic peptide
  PEPTIDE (1)..(1): K, H, or R
  misc_feature (3)..(3): Xaa can be any naturally occurring amino acid
  PEPTIDE (5)..(5): K, H, or R
XCXCX

SEQ ID NO: 695 - protein, 7 aa, Artificial Sequence
  synthetic peptide
  PEPTIDE (1)..(1): K, H, or R
  misc_feature (3)..(5): Xaa can be any naturally occurring amino acid
  PEPTIDE (7)..(7): K, H, or R
XCXXXCX

SEQ ID NO: 696 - protein, 4 aa, Artificial Sequence
  synthetic peptide
  PEPTIDE (1)..(1): K, H, or R
  PEPTIDE (3)..(3): K, H, or R
XCXC

SEQ ID NO: 697 - protein, 4 aa, Artificial Sequence
  synthetic peptide
  PEPTIDE (1)..(1): K, H, or R
  misc_feature (2)..(2): Xaa can be any naturally occurring amino acid
  PEPTIDE (4)..(4): K, H, or R
CXCX

SEQ ID NO: 698 - protein, 6 aa, Artificial Sequence
  synthetic peptide
  PEPTIDE (1)..(1): K, H, or R
  misc_feature (3)..(4): Xaa can be any naturally occurring amino acid
  PEPTIDE (5)..(5): K, H, or R
XCXXXC

SEQ ID NO: 699 - protein, 6 aa, Artificial Sequence
  synthetic peptide
  PEPTIDE (1)..(1): K, H, or R
  misc_feature (3)..(3): Xaa can be any naturally occurring amino acid
  PEPTIDE (4)..(4): K, H, or R
  misc_feature (5)..(5): Xaa can be any naturally occurring amino acid
XCXXXC

SEQ ID NO: 700 - protein, 6 aa, Artificial Sequence
  synthetic peptide
  PEPTIDE (1)..(1): K, H, or R
  PEPTIDE (3)..(3): K, H, or R
  misc_feature (4)..(5): Xaa can be any naturally occurring amino acid
XCXXXC

SEQ ID NO: 701 - protein, 6 aa, Artificial Sequence
  synthetic peptide
  misc_feature (2)..(3): Xaa can be any naturally occurring amino acid
  PEPTIDE (4)..(4): K, H, or R
  PEPTIDE (6)..(6): K, H, or R
CXXXCX

SEQ ID NO: 702 - protein, 6 aa, Artificial Sequence
  synthetic peptide
  misc_feature (2)..(2): Xaa can be any naturally occurring amino acid
  PEPTIDE (3)..(3): K, H, or R
  misc_feature (4)..(4): Xaa can be any naturally occurring amino acid
  PEPTIDE (6)..(6): K, H, or R
CXXXCX

SEQ ID NO: 703 - protein, 6 aa, Artificial Sequence
  synthetic peptide
  PEPTIDE (2)..(2): K, H, or R
  misc_feature (3)..(4): Xaa can be any naturally occurring amino acid
  PEPTIDE (6)..(6): K, H, or R
CXXXCX

SEQ ID NO: 704 - protein, 9 aa, Artificial Sequence
  synthetic peptide
YRSPFSRVV

SEQ ID NO: 705 - protein, 4 aa, Artificial Sequence
  synthetic peptide
HLYR

SEQ ID NO: 706 - protein, 19 aa, Artificial Sequence
  synthetic peptide
CPYCGWYRSPFSRVVHLYR

SEQ ID NO: 707 - protein, 20 aa, Artificial Sequence
  synthetic peptide
HCPYCGWYRSPFSRVVHLYR

SEQ ID NO: 708 - protein, 20 aa, Artificial Sequence
  synthetic peptide
HAPYAGWYRSPFSRVVHLYR

SEQ ID NO: 709 - protein, 18 aa, Artificial Sequence
  synthetic peptide
KCCGWYRSPFSRVVHLYR

SEQ ID NO: 710 - protein, 18 aa, Artificial Sequence
  synthetic peptide
KAAGWYRSPFSRVVHLYR

SEQ ID NO: 711 - protein, 17 aa, Artificial Sequence
  synthetic peptide
CCGWYRSPFSRVVHLYR

SEQ ID NO: 712 - protein, 19 aa, Artificial Sequence
  synthetic peptide
KCRCGWYRSPFSRVVHLYR

SEQ ID NO: 713 - protein, 19 aa, Artificial Sequence
  synthetic peptide
KARAGWYRSPFSRVVHLYR

SEQ ID NO: 714 - protein, 18 aa, Artificial Sequence
  synthetic peptide
CRCGWYRSPFSRVVHLYR

SEQ ID NO: 715 - protein, 18 aa, Artificial Sequence
  synthetic peptide
ARAGWYRSPFSRVVHLYR

SEQ ID NO: 716 - protein, 21 aa, Artificial Sequence
  synthetic peptide
KCRPYCGWYRSPFSRVVHLYR

SEQ ID NO: 717 - protein, 21 aa, Artificial Sequence
  synthetic peptide
KARPYAGWYRSPFSRVVHLYR

SEQ ID NO: 718 - protein, 20 aa, Artificial Sequence
  synthetic peptide
CRPYCGWYRSPFSRVVHLYR

SEQ ID NO: 719 - protein, 20 aa, Artificial Sequence
  synthetic peptide
ARPYAGWYRSPFSRVVHLYR

SEQ ID NO: 720 - protein, 21 aa, Artificial Sequence
  synthetic peptide
MEVGWYRSPFSRVVHLYRNGK

SEQ ID NO: 721 - protein, 20 aa, Artificial Sequence
  synthetic peptide
HCPYCNGGFNSNRANSSRSS

SEQ ID NO: 722 - protein, 19 aa, Artificial Sequence
  synthetic peptide
HCPYCLALWEPKPTQAFVK

SEQ ID NO: 723 - protein, 4 aa, Artificial Sequence
  synthetic peptide
  PEPTIDE (1)..(1): C, S, or T
  misc_feature (2)..(3): Xaa can be any naturally occurring amino acid
XXXC

SEQ ID NO: 724 - protein, 5 aa, Artificial Sequence
  synthetic peptide
  PEPTIDE (1)..(1): C, S, or T
  misc_feature (2)..(4): Xaa can be any naturally occurring amino acid
XXXXC

SEQ ID NO: 725 - protein, 6 aa, Artificial Sequence
  synthetic peptide
  PEPTIDE (1)..(1): C, S, or T
  misc_feature (2)..(5): Xaa can be any naturally occurring amino acid
XXXXXC

SEQ ID NO: 726 - protein, 7 aa, Artificial Sequence
  synthetic peptide
  PEPTIDE (1)..(1): C, S, or T
  misc_feature (2)..(6): Xaa can be any naturally occurring amino acid
XXXXXXC

SEQ ID NO: 727 - protein, 8 aa, Artificial Sequence
  synthetic peptide
  PEPTIDE (1)..(1): C, S, or T
  misc_feature (2)..(7): Xaa can be any naturally occurring amino acid
XXXXXXXC

SEQ ID NO: 728 - protein, 5 aa, Artificial Sequence
  synthetic peptide
  misc_feature (1)..(1): Xaa can be any naturally occurring amino acid
  PEPTIDE (2)..(2): C, S, or T
  misc_feature (3)..(4): Xaa can be any naturally occurring amino acid
XXXXC

SEQ ID NO: 729 - protein, 6 aa, Artificial Sequence
  synthetic peptide
  misc_feature (1)..(1): Xaa can be any naturally occurring amino acid
  PEPTIDE (2)..(2): C, S, or T
  misc_feature (3)..(5): Xaa can be any naturally occurring amino acid
XXXXXC

SEQ ID NO: 730 - protein, 7 aa, Artificial Sequence
  synthetic peptide
  misc_feature (1)..(1): Xaa can be any naturally occurring amino acid
  PEPTIDE (2)..(2): C, S, or T
  misc_feature (3)..(6): Xaa can be any naturally occurring amino acid
XXXXXXC

SEQ ID NO: 731 - protein, 8 aa, Artificial Sequence
  synthetic peptide
  misc_feature (1)..(1): Xaa can be any naturally occurring amino acid
  PEPTIDE (2)..(2): C, S, or T
  misc_feature (3)..(7): Xaa can be any naturally occurring amino acid
XXXXXXXC

SEQ ID NO: 732 - protein, 9 aa, Artificial Sequence
  synthetic peptide
  misc_feature (1)..(1): Xaa can be any naturally occurring amino acid
  PEPTIDE (2)..(2): C, S, or T
  misc_feature (3)..(8): Xaa can be any naturally occurring amino acid
XXXXXXXXC

SEQ ID NO: 733 - protein, 4 aa, Artificial Sequence
  synthetic peptide
  misc_feature (1)..(2): Xaa can be any naturally occurring amino acid
  PEPTIDE (3)..(3): C, S, or T
XXXC

SEQ ID NO: 734 - protein, 5 aa, Artificial Sequence
  synthetic peptide
  misc_feature (1)..(2): Xaa can be any naturally occurring amino acid
  PEPTIDE (3)..(3): C, S, or T
  misc_feature (4)..(4): Xaa can be any naturally occurring amino acid
XXXXC

SEQ ID NO: 735 - protein, 6 aa, Artificial Sequence
  synthetic peptide
  misc_feature (1)..(2): Xaa can be any naturally occurring amino acid
  PEPTIDE (3)..(3): C, S, or T
  misc_feature (4)..(5): Xaa can be any naturally occurring amino acid
XXXXXC

SEQ ID NO: 736 - protein, 7 aa, Artificial Sequence
  synthetic peptide
  misc_feature (1)..(2): Xaa can be any naturally occurring amino acid
  PEPTIDE (3)..(3): C, S, or T
  misc_feature (4)..(6): Xaa can be any naturally occurring amino acid
XXXXXXC

SEQ ID NO: 737 - protein, 8 aa, Artificial Sequence
  synthetic peptide
  misc_feature (1)..(2): Xaa can be any naturally occurring amino acid
  PEPTIDE (3)..(3): C, S, or T
  misc_feature (4)..(7): Xaa can be any naturally occurring amino acid
XXXXXXXC

SEQ ID NO: 738 - protein, 9 aa, Artificial Sequence
  synthetic peptide
  misc_feature (1)..(2): Xaa can be any naturally occurring amino acid
  PEPTIDE (3)..(3): C, S, or T
  misc_feature (4)..(8): Xaa can be any naturally occurring amino acid
XXXXXXXXC

SEQ ID NO: 739 - protein, 10 aa, Artificial Sequence
  synthetic peptide
  misc_feature (1)..(2): Xaa can be any naturally occurring amino acid
  PEPTIDE (3)..(3): C, S, or T
  misc_feature (4)..(9): Xaa can be any naturally occurring amino acid
XXXXXXXXXC

SEQ ID NO: 740 - protein, 4 aa, Artificial Sequence
  synthetic peptide
  misc_feature (2)..(3): Xaa can be any naturally occurring amino acid
  PEPTIDE (4)..(4): C, S, or T
CXXX

SEQ ID NO: 741 - protein, 5 aa, Artificial Sequence
  synthetic peptide
  misc_feature (2)..(4): Xaa can be any naturally occurring amino acid
  PEPTIDE (5)..(5): C, S, or T
CXXXX

SEQ ID NO: 742 - protein, 6 aa, Artificial Sequence
  synthetic peptide
  misc_feature (2)..(5): Xaa can be any naturally occurring amino acid
  PEPTIDE (6)..(6): C, S, or T
CXXXXX

SEQ ID NO: 743 - protein, 7 aa, Artificial Sequence
  synthetic peptide
  misc_feature (2)..(6): Xaa can be any naturally occurring amino acid
  PEPTIDE (7)..(7): C, S, or T
CXXXXXX

SEQ ID NO: 744 - protein, 8 aa, Artificial Sequence
  synthetic peptide
  misc_feature (2)..(7): Xaa can be any naturally occurring amino acid
  PEPTIDE (8)..(8): C, S, or T
CXXXXXXX

SEQ ID NO: 745 - protein, 5 aa, Artificial Sequence
  synthetic peptide
  misc_feature (1)..(1): Xaa can be any naturally occurring amino acid
  misc_feature (3)..(4): Xaa can be any naturally occurring amino acid
  PEPTIDE (5)..(5): C, S, or T
XCXXX

SEQ ID NO: 746 - protein, 6 aa, Artificial Sequence
  synthetic peptide
  misc_feature (1)..(1): Xaa can be any naturally occurring amino acid
  misc_feature (3)..(5): Xaa can be any naturally occurring amino acid
  PEPTIDE (6)..(6): C, S, or T
XCXXXX

SEQ ID NO: 747 - protein, 7 aa, Artificial Sequence
  synthetic peptide
  misc_feature (1)..(1): Xaa can be any naturally occurring amino acid
  misc_feature (3)..(6): Xaa can be any naturally occurring amino acid
  PEPTIDE (7)..(7): C, S, or T
XCXXXXX

SEQ ID NO: 748 - protein, 8 aa, Artificial Sequence
  synthetic peptide
  misc_feature (1)..(1): Xaa can be any naturally occurring amino acid
  misc_feature (3)..(7): Xaa can be any naturally occurring amino acid
  PEPTIDE (8)..(8): C, S, or T
XCXXXXXX

SEQ ID NO: 749 - protein, 9 aa, Artificial Sequence
  synthetic peptide
  misc_feature (1)..(1): Xaa can be any naturally occurring amino acid
  misc_feature (3)..(8): Xaa can be any naturally occurring amino acid
  PEPTIDE (9)..(9): C, S, or T
XCXXXXXXX

SEQ ID NO: 750 - protein, 4 aa, Artificial Sequence
  synthetic peptide
  misc_feature (1)..(2): Xaa can be any naturally occurring amino acid
  PEPTIDE (4)..(4): C, S, or T
XXCX

SEQ ID NO: 751 - protein, 5 aa, Artificial Sequence
  synthetic peptide
  misc_feature (1)..(2): Xaa can be any naturally occurring amino acid
  misc_feature (4)..(4): Xaa can be any naturally occurring amino acid
  PEPTIDE (5)..(5): C, S, or T
XXCXX

SEQ ID NO: 752 - protein, 6 aa, Artificial Sequence
  synthetic peptide
  misc_feature (1)..(2): Xaa can be any naturally occurring amino acid
  misc_feature (4)..(5): Xaa can be any naturally occurring amino acid
  PEPTIDE (6)..(6): C, S, or T
XXCXXX

SEQ ID NO: 753 - protein, 7 aa, Artificial Sequence
  synthetic peptide
  misc_feature (1)..(2): Xaa can be any naturally occurring amino acid
  misc_feature (4)..(6): Xaa can be any naturally occurring amino acid
  PEPTIDE (7)..(7): C, S, or T
XXCXXXX

SEQ ID NO: 754 - protein, 8 aa, Artificial Sequence
  synthetic peptide
  misc_feature (1)..(2): Xaa can be any naturally occurring amino acid
  misc_feature (4)..(7): Xaa can be any naturally occurring amino acid
  PEPTIDE (8)..(8): C, S, or T
XXCXXXXX

SEQ ID NO: 755 - protein, 9 aa, Artificial Sequence
  synthetic peptide
  misc_feature (1)..(2): Xaa can be any naturally occurring amino acid
  misc_feature (4)..(8): Xaa can be any naturally occurring amino acid
  PEPTIDE (9)..(9): C, S, or T
XXCXXXXXX

SEQ ID NO: 756 - protein, 10 aa, Artificial Sequence
  synthetic peptide
  misc_feature (1)..(2): Xaa can be any naturally occurring amino acid
  misc_feature (4)..(9): Xaa can be any naturally occurring amino acid
  PEPTIDE (10)..(10): C, S, or T
XXCXXXXXXX

SEQ ID NO: 757 - protein, 4 aa, Artificial Sequence
  synthetic peptide
  PEPTIDE (1)..(1): C, S, or T
  misc_feature (2)..(3): Xaa can be any naturally occurring amino acid
XXXC

SEQ ID NO: 758 - protein, 4 aa, Artificial Sequence
  synthetic peptide
  misc_feature (2)..(3): Xaa can be any naturally occurring amino acid
  PEPTIDE (4)..(4): C, S, or T
CXXX

SEQ ID NO: 759 - protein, 5 aa, Artificial Sequence
  synthetic peptide
  PEPTIDE (1)..(1): C, S, or T
  misc_feature (2)..(4): Xaa can be any naturally occurring amino acid
XXXXC

SEQ ID NO: 760 - protein, 5 aa, Artificial Sequence
  synthetic peptide
  misc_feature (2)..(4): Xaa can be any naturally occurring amino acid
  PEPTIDE (5)..(5): C, S, or T
CXXXX

SEQ ID NO: 761 - protein, 6 aa, Artificial Sequence
  synthetic peptide
  PEPTIDE (1)..(1): C, S, or T
  misc_feature (2)..(5): Xaa can be any naturally occurring amino acid
XXXXXC

SEQ ID NO: 762 - protein, 6 aa, Artificial Sequence
  synthetic peptide
  misc_feature (2)..(5): Xaa can be any naturally occurring amino acid
  PEPTIDE (6)..(6): C, S, or T
CXXXXX

SEQ ID NO: 763 - protein, 7 aa, Artificial Sequence
  synthetic peptide
  PEPTIDE (1)..(1): C, S, or T
  misc_feature (2)..(6): Xaa can be any naturally occurring amino acid
XXXXXXC

SEQ ID NO: 764 - protein, 7 aa, Artificial Sequence
  synthetic peptide
  misc_feature (2)..(6): Xaa can be any naturally occurring amino acid
  PEPTIDE (7)..(7): C, S, or T
CXXXXXX

SEQ ID NO: 765 - protein, 8 aa, Artificial Sequence
  synthetic peptide
  PEPTIDE (1)..(1): C, S, or T
  misc_feature (2)..(7): Xaa can be any naturally occurring amino acid
XXXXXXXC

SEQ ID NO: 766 - protein, 8 aa, Artificial Sequence
  synthetic peptide
  misc_feature (2)..(7): Xaa can be any naturally occurring amino acid
  PEPTIDE (8)..(8): C, S, or T
CXXXXXXX

SEQ ID NO: 767 - protein, 21 aa, Artificial Sequence
  synthetic peptide
MHCPYCGWYRSPFSRVVHLYR

SEQ ID NO: 768 - DNA, 63 bp, Artificial Sequence
  synthetic mRNA
atgcactgtccctattgcggttggtaccgttctcccttctcaagagtggttcacctctaccga

SEQ ID NO: 769 - DNA, 63 bp, Artificial Sequence
  synthetic mRNA
atggaggtgggttggtaccgttctcccttctcaagagtggttcacctctaccgaaatggcaag

SEQ ID NO: 770 - protein, 4 aa, Artificial Sequence
  synthetic peptide
  PEPTIDE (1)..(1): C, S, or T and modified with an acetyl, methyl, ethyl, or propionyl group
  misc_feature (2)..(3): Xaa can be any naturally occurring amino acid
XXXC

SEQ ID NO: 771 - protein, 5 aa, Artificial Sequence
  synthetic peptide
  PEPTIDE (1)..(1): C, S, or T and modified with an acetyl, methyl, ethyl, or propionyl group
  misc_feature (2)..(4): Xaa can be any naturally occurring amino acid
XXXXC

SEQ ID NO: 772 - protein, 6 aa, Artificial Sequence
  synthetic peptide
  PEPTIDE (1)..(1): C, S, or T and modified with an acetyl, methyl, ethyl, or propionyl group
  misc_feature (2)..(5): Xaa can be any naturally occurring amino acid
XXXXXC

SEQ ID NO: 773 - protein, 7 aa, Artificial Sequence
  synthetic peptide
  PEPTIDE (1)..(1): C, S, or T and modified with an acetyl, methyl, ethyl, or propionyl group
  misc_feature (2)..(6): Xaa can be any naturally occurring amino acid
XXXXXXC

SEQ ID NO: 774 - protein, 8 aa, Artificial Sequence
  synthetic peptide
  PEPTIDE (1)..(1): C, S, or T and modified with an acetyl, methyl, ethyl, or propionyl group
  misc_feature (2)..(7): Xaa can be any naturally occurring amino acid
XXXXXXXC

SEQ ID NO: 775 - protein, 4 aa, Artificial Sequence
  synthetic peptide
  PEPTIDE (1)..(1): C that is modified with an acetyl, methyl, ethyl or propionyl group
  misc_feature (2)..(3): Xaa can be any naturally occurring amino acid
  PEPTIDE (4)..(4): C, S, or T
XXXX

SEQ ID NO: 776 - protein, 5 aa, Artificial Sequence
  synthetic peptide
  PEPTIDE (1)..(1): C that is modified with an acetyl, methyl, ethyl or propionyl group
  misc_feature (2)..(4): Xaa can be any naturally occurring amino acid
  PEPTIDE (5)..(5): C, S, or T
XXXXX

SEQ ID NO: 777 - protein, 6 aa, Artificial Sequence
  synthetic peptide
  PEPTIDE (1)..(1): C that is modified with an acetyl, methyl, ethyl or propionyl group
  misc_feature (2)..(5): Xaa can be any naturally occurring amino acid
  PEPTIDE (6)..(6): C, S, or T
XXXXXX

SEQ ID NO: 778 - protein, 7 aa, Artificial Sequence
  synthetic peptide
  PEPTIDE (1)..(1): C that is modified with an acetyl, methyl, ethyl or propionyl group
  misc_feature (2)..(6): Xaa can be any naturally occurring amino acid
  PEPTIDE (7)..(7): C, S, or T
XXXXXXX

SEQ ID NO: 779 - protein, 8 aa, Artificial Sequence
  synthetic peptide
  PEPTIDE (1)..(1): C that is modified with an acetyl, methyl, ethyl or propionyl group
  misc_feature (2)..(7): Xaa can be any naturally occurring amino acid
  PEPTIDE (8)..(8): C, S, or T
XXXXXXXX

SEQ ID NO: 780 - protein, 4 aa, Artificial Sequence
  synthetic peptide
  PEPTIDE (1)..(1): C, S, or T
  misc_feature (2)..(3): Xaa can be any naturally occurring amino acid
  PEPTIDE (4)..(4): C that is modified with an acetyl, methyl, ethyl, or propionyl group
XXXX

SEQ ID NO: 781 - protein, 5 aa, Artificial Sequence
  synthetic peptide
  PEPTIDE (1)..(1): C, S, or T
  misc_feature (2)..(4): Xaa can be any naturally occurring amino acid
  PEPTIDE (5)..(5): C that is modified with an acetyl, methyl, ethyl, or propionyl group
XXXXX

SEQ ID NO: 782 - protein, 6 aa, Artificial Sequence
  synthetic peptide
  PEPTIDE (1)..(1): C, S, or T
  misc_feature (2)..(5): Xaa can be any naturally occurring amino acid
  PEPTIDE (6)..(6): C that is modified with an acetyl, methyl, ethyl, or propionyl group
XXXXXX

SEQ ID NO: 783 - protein, 7 aa, Artificial Sequence
  synthetic peptide
  PEPTIDE (1)..(1): C, S, or T
  misc_feature (2)..(6): Xaa can be any naturally occurring amino acid
  PEPTIDE (7)..(7): C that is modified with an acetyl, methyl, ethyl, or propionyl group
XXXXXXX

SEQ ID NO: 784 - protein, 8 aa, Artificial Sequence
  synthetic peptide
  PEPTIDE (1)..(1): C, S, or T
  misc_feature (2)..(7): Xaa can be any naturally occurring amino acid
  PEPTIDE (8)..(8): C that is modified with an acetyl, methyl, ethyl, or propionyl group
XXXXXXXX

SEQ ID NO: 785 - protein, 4 aa, Artificial Sequence
  synthetic peptide
  misc_feature (2)..(3): Xaa can be any naturally occurring amino acid
  PEPTIDE (4)..(4): C, S, or T and modified with an acetyl, methyl, ethyl, or propionyl group
CXXX

SEQ ID NO: 786 - protein, 5 aa, Artificial Sequence
  synthetic peptide
  misc_feature (2)..(4): Xaa can be any naturally occurring amino acid
  PEPTIDE (5)..(5): C, S, or T and modified with an acetyl, methyl, ethyl, or propionyl group
CXXXX

SEQ ID NO: 787 - protein, 6 aa, Artificial Sequence
  synthetic peptide
  misc_feature (2)..(5): Xaa can be any naturally occurring amino acid
  PEPTIDE (6)..(6): C, S, or T and modified with an acetyl, methyl, ethyl, or propionyl group
CXXXXX

SEQ ID NO: 788 - protein, 7 aa, Artificial Sequence
  synthetic peptide
  misc_feature (2)..(6): Xaa can be any naturally occurring amino acid
  PEPTIDE (7)..(7): C, S, or T and modified with an acetyl, methyl, ethyl, or propionyl group
CXXXXXX

SEQ ID NO: 789 - protein, 8 aa, Artificial Sequence
  synthetic peptide
  misc_feature (2)..(7): Xaa can be any naturally occurring amino acid
  PEPTIDE (8)..(8): C, S, or T and modified with an acetyl, methyl, ethyl, or propionyl group
CXXXXXXX

SEQ ID NO: 790 - DNA, 63 bp, Artificial Sequence
  synthetic mRNA
atgcactgtccctattgcagcctgcagcccttggccctggaggggtccctgcagaagcgtggc

SEQ ID NO: 791 - protein, 21 aa, Artificial Sequence
  synthetic peptide
MHCPYCSLQPLALEGSLQKRG

SEQ ID NO: 792 - DNA, 72 bp, Artificial Sequence
  synthetic mRNA
atgaagcactgtccctattgcgtgaggtactttctgagggtgcccagctggaagataaccctgtttaaaaag

SEQ ID NO: 793 - protein, 24 aa, Artificial Sequence
  synthetic peptide
MKHCPYCVRYFLRVPSWKITLFKK

(19) United States
(12) Patent Application Publication
(10) Pub. No.: US 2025/0277223 A1
(43) Pub. Date: Sep. 4, 2025

(54) METHODS OF TREATING OR PREVENTING AUTOIMMUNE DISEASES

(71) Applicant: IMCYSE SA, Angleur (Liège) (BE)

(72) Inventor: Geoffrey GLOIRE, Liège (BE)

(21) Appl. No.: 18/563,288

(22) PCT Filed: May 25, 2022
(86) PCT No.: PCT/EP2022/064145

§ 371 Date: Nov. 21, 2023

(30) Foreign Application Priority Data
May 26, 2021 (EP) 21176003.8

(51) Int. Cl. C12N 15/62; A61K 9/00; A61K 9/50; A61K 31/7115; A61K 38/16; A61P 37/06

(52) U.S. Cl. CPC C12N 15/62; A61K 9/0019; A61K 9/5089; A61K 31/7115; A61K 38/16; A61P 37/06; C12N 2310/335

(57) ABSTRACT

The invention relates to non-immunogenic mRNA encoding immunogenic peptides comprising a T-cell epitope and an oxidoreductase motifs, and their use in the treatment and/or prevention of e.g. type-1 diabetes (T1D), multiple sclerosis (MS), neuromyelitis optica (NMO), or rheumatoid arthritis (RA) in subjects.

### CROSS-REFERENCE TO RELATED APPLICATIONS

This application is the U.S. national phase of International Application No. PCT/EP2022/064145 filed May 25, 2022 which designated the U.S. and claims priority to EP 21176003.8 filed May 26, 2021, the entire contents of each of which are hereby incorporated by reference.

### REFERENCE TO SEQUENCE LISTING SUBMITTED ELECTRONICALLY

The content of the electronically submitted sequence listing (Name: 2752-146_Sequence_Listing.txt, Size: 170,667 bytes, and Date of Creation: May 27, 2024) is herein incorporated by reference in its entirety.

### FIELD OF THE INVENTION

The present invention is situated in the medical field and provides methods of treating auto-immune diseases making use of non-immunogenic mRNA encoding peptides comprising a T-cell epitope of an autoantigen and an oxidoreductase motif.

### BACKGROUND OF THE INVENTION

Several strategies have been described to prevent the generation of an unwanted immune response against an antigen. WO2008/017517 describes a new strategy using peptides comprising an MHC class II epitope of a given antigenic protein and an oxidoreductase motif. These peptides convert CD4+ T cells into a cell type with cytolytic properties called cytolytic CD4+ T cells (cCD4). These cells are capable to kill via triggering apoptosis those antigen presenting cells (APC), which present the antigen from which the peptide is derived. WO2008/017517 demonstrates this concept for allergies and auto-immune diseases such as type I diabetes. Herein insulin can act as an autoantigen.

WO2009101207 and Carlier et al. (2012) Plos one 7, 10 e45366 further describe the antigen specific cytolytic cells in more detail.

WO2016059236, WO2020099356 and WO2020099352 disclose modified peptides comprising different improved types of oxidoreductase motifs.

In addition to the peptides comprising an MHC class II epitope of an allergen or antigen, WO2012069568 further disclosed the possibility of using NKT cell epitopes, binding the CD1d receptor and resulting in activation of cytolytic antigen-specific NKT cells, which have been shown to eliminate, in an antigen-specific manner, APC presenting said specific antigen.

WO2018188730 discloses non-immunogenic RNAs for the treatment of autoimmune 5 diseases.

In order to improve the efficacy of a treatment using peptides comprising a T cell epitope of an antigen and an oxidoreductase motif, the search for new ways of administering such peptides continues.

### SUMMARY OF THE INVENTION

The present invention provides a new manner of administrating peptides comprising a T-cell epitope of an antigen and an oxidoreductase motif, in particular to treat autoimmune diseases. The inventors have found that rather than administering the peptides themselves, it is also possible to administer non-immunogenic RNA encoding said peptides to patients suffering from e.g. autoimmune diseases. This was unexpected because according to the literature on administration of non-immunogenic mRNA coding for disease-related autoantigen to treat auto-immune diseases like experimental autoimmune encephalomyelitis (EAE) in mice (reviewed in e.g. Wardell and Levings 2021, Nature Biotechnology volume 39, pages 419-421), the latter induce immune suppression via the induction of regulatory T-cells (Tregs), while the redox-epitope fusion peptides are believed to act through the creation of antigen-specific cytolytic CD4+ T-cells (cCD4) killing antigen presenting cells (APCs) expressing said antigen and thereby repressing the immune-response towards said antigen. It was hence surprising to see that when using non-immunogenic mRNA delivery, said cytolytic T-cell response could also be induced. Activation of Tregs or inducing a population of cCD4 cells is indeed a completely different tolerizing pathway in the immune system. Furthermore, in a comparative MOG-induced mouse EAE model system experiment using on the one hand a non-immunogenic mRNA encoding a fragment of the MOG autoantigen known to induce immunosuppression by Tregs induction, and on the other hand a non-immunogenic mRNA encoding said same fragment of the MOG autoantigen fused to an oxidoreductase motif, the latter had an improved reducing effect on clinical EAE manifestation. Also, this was unexpected since it was not certain that the oxidoreductase motif upon translation in vivo would reach the same effect as when administered as a fusion peptide in vivo.

The inventor hence provides a proof of concept of using non-immunogenic mRNA encoding a fusion peptide comprising an oxidoreductase motif coupled to a T-cell epitope of an antigen in order to reduce immune response against said antigen.

The present invention therefore relates to the following aspects:
1. A method of treating or preventing a disease or condition selected from an autoimmune disease, an infection with an intracellular pathogen, a tumor, an allograft rejection, or an immune response to a soluble allofactor, to an allergen exposure or to a viral vector used for gene therapy or gene vaccination in a subject, comprising administering to the subject a non-immunogenic RNA encoding a peptide, said peptide comprising: a) an oxidoreductase motif;
b) a T-cell epitope of an antigenic protein involved in said disease or condition; and
c) a linker between a) and b) of between 0 and 7 amino acids, preferably of between 0 and 4 amino acids;
wherein said oxidoreductase motif a) has the following general structure:

Zₘ—[CST]-Xₙ—C— or Zₘ—C—Xₙ—[CST]-;
wherein Z is any amino acid, preferably a basic amino acid, more preferably selected from the group comprising: K, H, R and a non-natural basic amino acid, preferably K or H; wherein m is an integer selected from the group comprising: 1, 0, or 2; wherein X is any amino acid, preferably a basic amino acid, more preferably selected from the group comprising: K, H, R and a non-natural basic amino acid, preferably R; wherein n is an integer selected from 0 to 6, preferably from 0 to 3, most preferably 2; wherein in all general structures disclosed herein, the hyphen (-) in said oxidoreductase motif indicates the point of attachment of the oxidoreductase motif to the N-terminal end of the linker or the T cell epitope, or to the C-terminal end of the linker or the T cell epitope.
2. A method of inducing cytolytic CD4+ T cells in a subject, comprising administering to the subject non-immunogenic RNA encoding a peptide comprising:
a) an oxidoreductase motif;
b) a T-cell epitope of an antigenic protein involved in said disease; and
c) a linker between a) and b) of between 0 and 7 amino acids, preferably of between 0 and 4 amino acids;
wherein said oxidoreductase motif a) has the following general structure:

Zₘ—[CST]-Xₙ—C— or Zₘ—C—Xₙ—[CST]-;
wherein Z is any amino acid, preferably a basic amino acid, more preferably selected 5 from the group comprising: K, H, R and a non-natural basic amino acid, preferably K or H; wherein m is an integer selected from the group comprising: 1, 0, or 2; wherein X is any amino acid, preferably a basic amino acid, more preferably selected from the group comprising: K, H, R and a non-natural basic amino acid, preferably R; wherein n is an integer selected from 0 to 6, preferably from 0 to 3, most preferably wherein n is 2,
wherein in all general structures disclosed herein, the hyphen (-) in said oxidoreductase motif indicates the point of attachment of the oxidoreductase motif to the N-terminal end of the linker or the epitope, or to the C-terminal end of the linker or the T cell epitope.

In any one of the embodiments, prone to the mRNA translation mechanism, an additional start-codon (atg) is generally included at the 5′ end of the DNA coding sequence used to generate the mRNA, hence adding a methionine (M) residue to the encoded peptides disclosed herein. Said methionine residue can in some cases be cleaved off in vivo due to processing of the peptide.

In any one of the embodiments, the peptides encoded by the non-immunogenic RNA according to the invention have a reducing activity on disulfide bridges of peptides or proteins.
3. The method of aspect 2, wherein the subject has an autoimmune disease.
4. The method of aspects 1 or 3, wherein the autoimmune disease is selected from the group comprising: type-1 diabetes (T1D), a demyelinating disorder such as multiple sclerosis (MS) or neuromyelitis optica (NMO), or in rheumatoid arthritis (RA).

Preferably said antigenic protein is an autoantigen, an allergen, a soluble allofactor, an alloantigen shed by the graft, an antigen of an intracellular pathogen, an antigen of a viral vector used for gene therapy or gene vaccination, a tumor-associated antigen or an allergen.

Exemplary antigens can be:
myelin antigens, neuronal antigens, and astrocyte-derived antigens, for example: Myelin Oligodendrocyte Glycoprotein (MOG), Myelin basic protein (MBP), Proteolipid protein (PLP), Oligodendrocyte-specific protein (OSP), myelin-associated antigen (MAG), myelin-associated oligodendrocyte basic protein (MOBP), and 2′,3′-cyclic-nucleotide 3′-phosphodiesterase (CNPase), S100P protein or transaldolase H autoantigens in case of MS (Riedhammer and Weissert, 2015; Front Immunol. 2015; 6: 322), preferably MOG, MBP, PLP and MOBP.
allergens such as those derived from pollen, spores, dust mites, and pet dander in case of asthma.
tumour or cancer associated antigens such as oncogenes, proto-oncogenes, viral proteins, surviving factors or clonotypic or idiotypic determinants in case of cancer. Specific examples are: MAGE (melanoma-associated gene) products were shown to be spontaneously expressed by tumour cells in the context of MHC class I determinants, and as such, recognised by CD8+ cytolytic T cells. However, MAGE-derived antigens, such as MAGE-3, are also expressed in MHC class II determinants and CD4+ specific T cells have been cloned from melanoma patients (Schutz et al. (2000) Cancer Research 60: 6272-6275; Schuler-Thurner et al. (2002) J. Exp. Med. 195: 1279-1288). Peptides presented by MHC class II determinants are known in the art. Other examples include the gp100 antigen expressed by the P815 mastocytoma and by melanoma cells (Lapointe (2001; J. Immunol. 167: 4758-4764; Cochlovius et al. (1999) Int. J. Cancer, 83: 547-554). Proto-oncogenes include a number of polypeptides and proteins which are preferentially expressed in tumours cells, and only minimally in healthy tissues. Cyclin D1 is cell cycle regulator which is involved in the G1 to S transition. High expression of cyclin D1 has been demonstrated in renal cell carcinoma, parathyroid carcinomas and multiple myeloma. A peptide encompassing residues 198 to 212 has been shown to carry a T cell epitope recognised in the context of MHC class II determinants (Dengiel et al. (2004) Eur. J. of Immunol. 34: 3644-3651). Survivin is one example of a factor inhibiting apoptosis, thereby conferring an expansion advantage to survivin-expressing cells. Survivin is aberrantly expressed in human cancers of epithelial and hematopoietic origins and not expressed in healthy adult tissues except the thymus, testis and placenta, and in growth-hormone stimulated hematopoietic progenitors and endothelial cells. Interestingly, survivin-specific CD8+ T cells are detectable in blood of melanoma patients. Survivin is expressed by a broad variety of malignant cell lines, including renal carcinoma, breast cancer, and multiple myeloma, but also in acute myeloid leukemia, and in acute and chronic lymphoid leukemia (Schmidt (2003) Blood 102: 571-576). Other examples on inhibitors of apoptosis are Bcl2 and spi6. Idiotypic determinants are presented by B cells in follicular lymphomas, multiple myeloma and some forms of leukemia, and by T cell lymphomas and some T cell leukemias. Idiotypic determinants are part of the antigen-specific receptor of either the B cell receptor (BCR) or the T cell receptor (TCR). Such determinants are essentially encoded by hypervariable regions of the receptor, corresponding to complementarity-determining regions (CDR) of either the VH or VL regions in B cells, or the CDR3 of the beta chain in T cells. As receptors are created by the random rearrangement of genes, they are unique to each individual. Peptides derived from idiotypic determinants are presented in MHC class II determinants (Baskar et al. (2004) J. Clin. Invest. 113: 1498-1510). Some tumours are associated with expression of virus-derived antigens. Thus, some forms of Hodgkin disease express antigens from the Epstein-Barr virus (EBV). Such antigens are expressed in both class I and class II determinants. CD8+ cytolytic T cells specific for EBV antigens can eliminate Hodgkin lymphoma cells (Bollard et a/. (2004) J. Exp. Med. 200: 1623-1633). Antigenic determinants such as LMP-1 and LMP-2 are presented by MHC class II determinants.
transplant-specific antigens in case of transplant rejection, which will obviously be dependent on the type of tissue or organ being transplanted. Examples can be tissues such as cornea, skin, bones (bone chips), vessels or fascia; organs such as kidney, heart, liver, lungs, pancreas or intestine; or even individual cells such as pancreatic islet cells, alpha cells, beta cells, muscle cells, cartilage cells, heart cells, brain cells, blood cells, bone marrow cells, kidney cells and liver cells. Specific exemplary antigens involved in transplantation rejection are minor histocompatibility antigens, major histocompatibility antigens or tissue-specific antigens. Where the alloantigenic protein is a major histocompatibility antigen, this is either an MHC class I-antigen or an MHC class II-antigen. An important point to keep in mind is the variability of the mechanisms by which alloantigen-specific T cells recognize cognate peptides at the surface of APC. Alloreactive T cells can recognize either alloantigen-determinants of the MHC molecule itself, an alloantigen peptide bound to a MHC molecule of either autogenic or allogeneic source, or a combination of residues located within the alloantigen-derived peptide and the MHC molecule, the latter being of autogenic or allogeneic origin. Examples of minor histocompatibility antigens are those derived from proteins encoded by the HY chromosome (H—Y antigens), such as Dby. Other examples can be found in, for instance, Goulmy E, Current Opinion in Immunology, vol 8, 75-81, 1996 (see Table 3 therein in particular). It has to be noted that many minor histocompatibility antigens in man have been detected via their presentation into MHC class I determinants by use of cytolytic CD8+ T cells. However, such peptides are derived by the processing of proteins that also contain MHC class II restricted T cell epitopes, thereby providing the possibility of designing peptides of the present invention. Tissue-specific alloantigens can be identified using the same procedure. One example of this is the MHC class I restricted epitope derived from a protein expressed in kidneys but not in spleen and capable of eliciting CD8+ T cells with cytotoxic activity on kidney cells (Poindexter et al, Journal of Immunology, 154: 3880-3887, 1995).

More preferably, said antigenic protein is an autoantigen involved in type-1 diabetes (T1D), demyelinating disorders such as multiple sclerosis (MS) or neuromyelitis optica (NMO), or in rheumatoid arthritis (RA).

### Non-Limiting Examples of Autoantigens Involved in T1D.

Source: Mallone R et al., Clin Dev Immunol. 2011:513210.

| | UniProtKB | Examples of T-cell |
|---|---|---|
| Auto-antigen | identifier | epitopes |
| Insulin | P01308 | LALEGSLQK |
| | | (SEQ ID NO: 3) |
| GAD65 | Q9UGI5 | |
| GAD67 | Q99259 | |
| IA-2 (ICA512) | Q16849 | |
| IA-2 (beta/phogrin) | Q92932 | |
| IGRP | Q9NQR9 | |
| Chromogranin | P10645 | |
| ZnT8 | Q8IWU4 | |
| HSP-60 | P10809 | |

### Non-Limiting Examples of Autoantigens Involved in MS.

| | UniProtKB | | Examples of T- |
|---|---|---|---|
| Auto-antigen | identifier | Comments | cell epitopes |
| Myelin oligodendrocyte | Q16653 | Immunodominant | FLRVPCWKI |
| glycoprotein (MOG) | | epitopes in the following | (SEQ ID NO: 4) |
| | | regions: amino acids 1- | or FLRVPSWKI |
| | | 22, 35-55 and 92-106. | (SEQ ID NO: 5) |
| Myelin basic protein | P02686 | Immunodominant | |
| (MBP) | | epitopes in the amino | |
| | | acids 85-99 region. | |
| Proteolipid protein | P60201 | Immunodominant | |
| (PLP) | | epitopes in the following | |
| | | regions: amino acids 31- | |
| | | 70, 91-120 and 178-228. | |
| Myelin-oligodendrocytic | Q13875 | Immunodominant | |
| basic protein (MOBP) | | epitopes in the amino | |
| | | acids 15-36 region. | |
| Oligodendrocyte- | O75508 | Immunodominant | |
| specific protein (OSP) | | epitopes in the amino | |
| | | acids 179-207 region. | |

### Non-Limiting Examples of Autoantigens Involved in RA.

| Auto-antigen | UniProtKB identifier |
|---|---|
| GRP78 | P11021 |
| HSP60 | P10809 |
| 60 kDa chaperonin 2 | P9WPE7 |
| Gelsolin | P06396 |
| Chitinase-3-like protein 1 | P36222 |
| Cathepsin S | P25774 |
| Serum albumin | P02768 |
| Cathepsin D | P07339 |

### Non-Limiting Examples of Autoantigens Involved in NMO

| | UniProtKB | | Examples of T- |
|---|---|---|---|
| Auto-antigen | identifier | Comments | cell epitopes |
| Myelin | Q16653 | Immunodominant | FLRVPCWKI (SEQ |
| oligodendrocyte | | epitopes in the following | ID NO: 4) or |
| glycoprotein (MOG) | | regions: amino acids 1- | FLRVPSWKI (SEQ |
| | | 22, 35-55 and 92-106. | ID NO: 5) |

5. The method of any one of aspects 1 to 4, wherein the non-immunogenic RNA is rendered non-immunogenic by the incorporation of modified nucleotides and the removal of dsRNA.
6. The method of aspect 5, wherein the modified nucleotides suppress RNA-mediated activation of innate immune receptors.
7. The method of aspect 5 or 6, wherein the modified nucleotides comprise a replacement of one or more uridines with a nucleoside comprising a modified nucleobase.
8. The method of aspect 7, wherein the modified nucleobase is a modified uracil.
9. The method of aspect 7 or 8, wherein the nucleoside comprising a modified nucleobase is selected from the group consisting of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine (τm5s2U), 1-taurinomethyl-4-thio-pseudouridine, 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N(1)-methyl-pseudouridine (m1ψ), 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine.
10. The method of any one of aspects 7 to 9, wherein the nucleoside comprising a modified nucleobase is pseudouridine (ψ), N(1)-methyl-pseudouridine (m1ψ) or 5-methyl-uridine (m5U).
11. The method of any one of aspects 7 to 10, wherein the nucleoside comprising a modified nucleobase is N(1)-methyl-pseudouridine (m1ψ).
12. The method of any one of aspects 1 to 11, wherein the non-immunogenic RNA is mRNA.
13. The method of any one of aspects 1 to 12, wherein the non-immunogenic RNA is in vitro transcribed RNA.
14. The method of any one of aspects 1 to 13, wherein the non-immunogenic RNA is transiently expressed in cells of the subject.
15. The method of any one of aspects 1 to 14, wherein the non-immunogenic RNA is delivered to dendritic cells.
16. The method of aspect 15, wherein the dendritic cells are immature dendritic cells.
17. The method of any one of aspects 1 to 16, wherein the non-immunogenic RNA is formulated in a delivery vehicle.
18. The method of aspect 17, wherein the delivery vehicle comprises particles.
19. The method of aspect 17 or 18, wherein the delivery vehicle comprises a lipid.
20. The method of aspect 19, wherein the lipid comprises a cationic lipid.
21. The method of aspect 19 or 20, wherein the lipid forms a complex with and/or encapsulates the non-immunogenic RNA.
22. The method of any one of aspects 1 to 21, wherein the non-immunogenic RNA is formulated in liposomes.
23. The method of any one of aspects 1 to 22, wherein said oxidoreductase motif is selected from the following amino acid motifs:

Zₘ—[CST]-Xₙ—C— or Zₘ—C—Xₙ—[CST]-, (a)
wherein n is 0, and wherein m is an integer selected from 0, 1, or 2,
wherein Z is a basic amino acid preferably selected from: H, K, R, and a non-natural basic amino acid as defined herein, such as L-ornithine, more preferably K or H, most preferably K. Non-limiting preferred examples of such motifs are KCC, KKCC (SEQ ID NO: 6), RCC, RRCC (SEQ ID NO: 7), RKCC (SEQ ID NO: 8), or KRCC (SEQ ID NO: 9);

Zₘ—[CST]-Xₙ—C— or Zₘ—C—Xₙ—[CST]-, (b)
wherein n is 1, wherein X is any amino acid, preferably a basic amino acid selected from: H, K, R, and a non-natural basic amino acid such as L-ornithine, more preferably K or R, wherein m is an integer selected from 0, 1, or 2,
wherein Z is a basic amino acid preferably selected from: H, K, R, and a non-natural basic amino acid as defined herein, such as L-ornithine, more preferably K or H, most preferably K. Non-limiting preferred examples of such motifs are KCXC (SEQ ID NO: 10), KKCXC (SEQ ID NO: 11), RCXC (SEQ ID NO: 12), RRCXC (SEQ ID NO: 13), RKCXC (SEQ ID NO:14), KRCXC (SEQ ID NO: 15), KCKC (SEQ ID NO: 16), KKCKC (SEQ ID NO: 17), KCRC (SEQ ID NO: 18), KKCRC (SEQ ID NO: 19), RCRC (SEQ ID NO: 20), RRCRC (SEQ ID NO: 21), RKCKC (SEQ ID NO: 22), KRCKC (SEQ ID NO: 23);
(c) Zₘ—[CST]-Xₙ—C— or Zₘ—C—Xₙ—[CST]-, wherein n is 2, thereby creating an internal X¹X² amino acid couple within the oxidoreductase motif, wherein X is any amino acid, preferably wherein at least one X is a basic amino acid selected from: H, K, R, and a non-natural basic amino acid such as L-ornithine, more preferably K or R,
wherein m is an integer selected from 0, 1, or 2,
wherein Z is a basic amino acid preferably selected from: H, K, R, and a non-natural basic amino acid as defined herein, such as L-ornithine, more preferably K or H, most preferably K. In these motifs, an internal X¹X² amino acid couple is situated within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2 and Z is a basic amino acid selected from: H, K, or R, or a non-natural basic amino acid as defined herein, such as L-ornithine, preferably K or H. X¹ and X², each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a further example, at least one of X¹ or X² in said motif is a basic amino acid selected from: H, K, or R, or a non-natural basic amino acid as defined herein, such as L-ornithine. In another example of the motif, at least one of X¹ or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Particularly preferred motifs of this form are [CST]XXC or CXX[CST](SEQ ID NO: 1 or 2), HCPYC, KHCPYC, KCPYC, RCPYC, HCGHC, KCGHC, and RCGHC (corresponding to SEQ ID NOs: 24 to 30). Alternative preferred motifs of this form are KKCPYC, KRCPYC, KHCGHC, KKCGHC, and KRCGHC (SEQ ID NOs: 31 to 35);
(d) Zₘ—[CST]-Xₙ—C— or Zₘ—C—Xₙ—[CST]-, wherein n is 3, thereby creating an internal X¹X²X³ amino acid stretch within the oxidoreductase motif, wherein X is any amino acid, preferably wherein at least one X is a basic amino acid selected from: H, K, R, and a non-natural basic amino acid such as L-ornithine, more preferably K or R,
wherein m is an integer selected from 0, 1, or 2,
wherein Z is a basic amino acid preferably selected from: H, K, R, and a non-natural basic amino acid as defined herein, such as L-ornithine, more preferably K or H. Preferred are motifs wherein m is 1 or 2 and Z is a basic amino acid selected from: H, K, or R, or a non-natural basic amino acid as defined herein, such as L-ornithine, preferably K or H. In certain examples, X¹, X², and X³, each individually can be any amino acid selected from the group consisting of: G, A, V, L, I, M, F, W, P, S, T, C, Y, N, Q, D, E, K, R, and H, or non-natural amino acids. Preferably, X¹, X², and X³ in said motif is any amino acid except for C, S, or T. In a specific embodiment, at least one of X¹, X², or X³ in said motif is a basic amino acid selected from: H, K, or R, or a non-natural basic amino acid as defined herein, such as L-ornithine. Specific examples of the internal X¹X²X³ amino acid stretch within the oxidoreductase motif are: XPY, PXY, and PYX, wherein X can be any amino acid, preferably a basic amino acid such as K, R, or H, or a non-natural basic amino acid such as L-ornithine. Non-limiting examples include KPY, RPY, HPY, GPY, APY, VPY, LPY, IPY, MPY, FPY, WPY, PPY, SPY, TPY, CPY, YPY, NPY, QPY, DPY, EPY, KPY, PKY, PRY, PHY, PGY, PAY, PVY, PLY, PIY, PMY, PFY, PWY, PPY, PSY, PTY, PCY, PYY, PNY, PQY, PDY, PEY, PLY, PYK, PYR, PYH, PYG, PYA, PYV, PYL, PYI, PYM, PYF, PYW, PYP, PYS, PYT, PYC, PYY, PYN, PYQ, PYD, or PYE. Alternative examples of the internal X¹X²X³ amino acid stretch within the oxidoreductase motif are XHG, HXG, and HGX, wherein X can be any amino acid, such as in KHG, RHG, HHG, GHG, AHG, VHG, LHG, IHG, MHG, FHG, WHG, PHG, SHG, THG, CHG, YHG, NHG, QHG, DHG, EHG, and KHG, HKG, HRG, HHG, HGG, HAG, HVG, HLG, HIG, HMG, HFG, HWG, HPG, HSG, HTG, HCG, HYG, HNG, HQG, HDG, HEG, HLG, HGK, HGR, HGH, HGG, HGA, HGV, HGL, HGI, HGM, HGF, HGW, HGP, HGS, HGT, HGC, HGY, HGN, HGQ, HGD, or HGE. Yet alternative examples of the internal X¹X²X³ amino acid stretch within the oxidoreductase motif are XGP, GXP, and GPX, wherein X can be any amino acid, such as in KGP, RGP, HGP, GGP, AGP, VGP, LGP, IGP, MGP, FGP, WGP, PGP, SGP, TGP, CGP, YGP, NGP, QGP, DGP, EGP, KGP, GKP, GRP, GHP, GGP, GAP, GVP, GLP, GIP, GMP, GFP, GWP, GPP, GSP, GTP, GCP, GYP, GNP, GQP, GDP, GEP, GLP, GPK, GPR, GPH, GPG, GPA, GPV, GPL, GPI, GPM, GPF, GPW, GPP, GPS, GPT, GPC, GPY, GPN, GPQ, GPD, or GPE. Yet alternative examples of the internal X¹X²X³ amino acid stretch within the oxidoreductase motif are XGH, GXH, and GHX, wherein X can be any amino acid, such as in KGH, RGH, HGH, GGH, AGH, VGH, LGH, IGH, MGH, FGH, WGH, PGH, SGH, TGH, CGH, YGH, NGH, QGH, DGH, EGH, KGH, GKH, GRH, GHH, GGH, GAH, GVH, GLH, GIH, GMH, GFH, GWH, GPH, GSH, GTH, GCH, GYH, GNH, GQH, GDH, GEH, GLH, GHK, GHR, GHH, GHG, GHA, GHV, GHL, GHI, GHM, GHF, GHW, GHP, GHS, GHT, GHC, GHY, GHN, GHQ, GHD, or GHE. Yet alternative examples of the internal X¹X²X³ amino acid stretch within the oxidoreductase motif are XGF, GXF, and GFX, wherein X can be any amino acid, such as in KGF, RGF, HGF, GGF, AGF, VGF, LGF, IGF, MGF, FGF, WGF, PGF, SGF, TGF, CGF, YGF, NGF, QGF, DGF, EGF, and KGF, GKF, GRF, GHF, GGF, GAF, GVF, GLF, GIF, GMF, GFF, GWF, GPF, GSF, GTF, GCF, GYF, GNF, GQF, GDF, GEF, GLF, GFK, GFR, GFH, GFG, GFA, GFV, GFL, GFI, GFM, GFF, GFW, GFP, GFS, GFT, GFC, GFY, GFN, GFQ, GFD, or GFE. Yet alternative examples of the internal X¹X²X³ amino acid stretch within the oxidoreductase motif are XRL, RXL, and RLX, wherein X can be any amino acid, such as in KRL, RRL, HRL, GRL, ARL, VRL, LRL, IRL, MRL, FRL, WRL, PRL, SRL, TRL, CRL, YRL, NRL, QRLRL, DRL, ERL, KRL, GKF, GRF, GHF, GGF, GAF, GVF, GLF, GIF, GMF, GFF, GWF, GPF, GSF, GTF, GCF, GYF, GNF, GQF, GDF, GEF, and GLF, RLK, RLR, RLH, RLG, RLA, RLV, RLL, RLI, RLM, RLF, RLW, RLP, RLS, RLT, RLC, RLY, RLN, RLQ, RLD, or RLE. Yet alternative examples of the internal X¹X²X³ amino acid stretch within the oxidoreductase motif are XHP, HXP, and HPX, wherein X can be any amino acid, such as in KHP, RHP, HHP, GHP, AHP, VHP, LHP, IHP, MHP, FHP, WHP, PHP, SHP, THP, CHP, YHP, NHP, QHP, DHP, EHP, KHP, HKP, HRP, HHP, HGP, HAF, HVF, HLF, HIF, HMF, HFF, HWF, HPF, HSF, HTF, HCF, HYP, HNF, HQF, HDF, HEF, HLP, HPK, HPR, HPH, HPG, HPA, HPV, HPL, HPI, HPM, HPF, HPW, HPP, HPS, HPT, HPC, HPY, HPN, HPQ, HPD, or HP^{E}.

Particularly preferred examples are: CRPYC, KCRPYC, KHCRPYC, RCRPYC, HCRPYC, CPRYC, KCPRYC, RCPRYC, HCPRYC, CPYRC, KCPYRC, RCPYRC, HCPYRC, CKPYC, KCKPYC, RCKPYC, HCKPYC, CPKYC, KCPKYC, RCPKYC, HCPKYC, CPYKC, KCPYKC, RCPYKC, and HCPYKC (corresponding to SEQ ID NOs: 36 to 60);
(e) Zₘ—[CST]-Xₙ—C— or Zₘ—C—Xₙ—[CST]-, wherein n is 4, thereby creating an internal X¹X²X³X⁴ (SEQ ID NO: 76) amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K. Preferred are motifs wherein m is 1 or 2 and Z is a basic amino acid selected from: H, K, or R, or a non-natural basic amino acid as defined herein, such as L-ornithine, preferably K or H. X¹, X², X³ and X⁴ each individually can be any amino acid selected from the group consisting of: G, A, V, L, I, M, F, W, P, S, T, C, Y, N, Q, D, E, K, R, and H, or non-natural amino acids as defined herein. Preferably, X¹, X², X³ and X⁴ in said motif is any amino acid except for C, S, or T. In certain non-limiting examples, at least one of X¹, X², X³ or X⁴ in said motif is a basic amino acid selected from: H, K, or R, or a non-natural basic amino acid as defined herein. Specific examples include LAVL (SEQ ID NO: 61), TVQA (SEQ ID NO: 62) or GAVH (SEQ ID NO: 63) and their variants such as: X¹AVL, LX²VL, LAX³L, or LAVX⁴; X¹VQA, TX²QA, TVX³A, or TVQX⁴; X¹AVH, GX²VH, GAX³H, or GAVX⁴ (corresponding to SEQ ID NO: 64 to 75); wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein;
(f) Zₘ—[CST]-Xₙ—C— or Zₘ—C—Xₙ—[CST]-, wherein n is 5, thereby creating an internal X¹X²X³X⁴X⁵ (SEQ ID NO: 77) amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K. Preferred are motifs wherein m is 1 or 2 and Z is a basic amino acid selected from: H, K, or R, or a non-natural basic amino acid as defined herein, such as L-ornithine, preferably K or H. X¹, X², X³, X⁴ and X⁵ each individually can be any amino acid selected from the group consisting of: G, A, V, L, I, M, F, W, P, S, T, C, Y, N, Q, D, E, K, R, and H, or non-natural amino acids. Preferably, X¹, X², X³, X⁴ and X⁵ in said motif is any amino acid except for C, S, or T. In certain examples, at least one of X¹, X², X³ X⁴ or X⁵ in said motif is a basic amino acid selected from: H, K, or R, or a non-natural basic amino acid as defined herein. Specific examples include PAFPL (SEQ ID NO: 78) or DQGGE (SEQ ID NO: 79) and their variants such as: X¹AFPL, PX²FPL, PAX³PL, PAFX⁴L, or PAFPX⁵; X¹QGGE, DX²GGE, DQX³GE, DQGX⁴E, or DQGGX⁵ (corresponding to SEQ ID NO: 80 to 89), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural amino acids as defined herein;
(g) Zₘ—[CST]-Xₙ—C— or Zₘ—C—Xₙ—[CST]-, wherein n is 6, thereby creating an internal X¹X²X³X⁴X⁵X⁶ (SEQ ID NO: 90) amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K. Preferred are motifs wherein m is 1 or 2 and Z is a basic amino acid selected from: H, K, or R, or a non-natural basic amino acid as defined herein, such as L-ornithine, preferably K or H. X¹, X², X³, X⁴ X⁵ and X⁶ each individually can be any amino acid selected from the group consisting of: G, A, V, L, I, M, F, W, P, S, T, C, Y, N, Q, D, E, K, R, and H, or non-natural amino acid. Preferably, X¹, X², X³, X⁴, X⁵ and X⁶ in said motif is any amino acid except for C, S, or T. In certain examples, at least one of X¹, X², X³ X⁴, X⁵ or X⁶ in said motif is a basic amino acid selected from: H, K, or R, or a non-natural basic amino acid as defined herein. Specific examples include DIADKY (SEQ ID NO: 91) or variants thereof such as: X¹IADKY, DX²ADKY, DIX³DKY, DIAX⁴KY, DIADX⁵Y, or DIADKX⁶ (corresponding to SEQ ID NO: 92 to 97), wherein X¹, X², X³, X⁴, X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein; or
(h) Zₘ—[CST]-Xₙ—C— or Zₘ—C—Xₙ—[CST]-, wherein n is 0 to 6 and wherein m is 0, and wherein one of the C or [CST] residues has been modified so as to carry an acetyl, methyl, ethyl or propionyl group, either on the N-terminal amide of the amino acid residue of the motif or on the C-terminal carboxy group. In preferred embodiments of such a motif, n is 2 and m is 0, wherein the internal X¹X², each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a further example, at least one of X¹ or X² in said motif is a basic amino acid selected from: H, K, or R, or a non-natural basic amino acid as defined herein, such as L-ornithine. In another example of the motif, at least one of X¹ or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Preferably said modification results in an N-terminal acetylation of the first cysteine in the motif (N-acetyl-cysteine).
25. The method according to any one of aspects 1 to 24, wherein the Xₒ portion of said oxidoreductase motif comprises the sequence PY, preferably wherein the oxidoreductase motif comprises the sequence CPYC (SEQ ID NO: 98).
26. The method according to any one of aspects 1 to 25, wherein amino acid Z of the oxidoreductase motif is a basic amino acid selected from the group of amino acids consisting of: H, K, R, and any non-natural basic amino acid, more preferably a basic amino acid selected from: H, K, and R, most preferably wherein Z is H or K.
27. The method of any one of aspects 1 to 26, wherein said immunogenic peptide has a length of between 9 and 50 amino acids, preferably of between 9 and 30 amino acids.
28. The method of any one of aspects 1 to 27, wherein said oxidoreductase motif does not naturally occur in the amino acid sequence within a region of 11 amino acids N-terminally or C-terminally of the T-cell epitope in said antigenic protein and/or wherein said the T-cell epitope does not naturally comprise said oxidoreductase motif in its amino acid sequence.
29. The method of any one of aspects 1 to 28, wherein said antigenic protein is selected from the group consisting of: (pro)insulin, GAD65, GAD67, IA-2 (ICA512), IA-2 (beta/phogrin), IGRP, Chromogranin, ZnT8 and HSP-60, and wherein said autoimmune disease is type-1 diabetes (T1D).
30. The method of any one of aspects 1 to 28, wherein said antigenic protein is selected from the group consisting of: Myelin oligodendrocyte glycoprotein (MOG), Myelin basic protein (MBP), Proteolipid protein (PLP), Myelin-oligodendrocytic basic protein (MOBP), and Oligodendrocyte-specific protein (OSP), and wherein said autoimmune disease is multiple sclerosis (MS), and/or neuromyelitis optica (NMO).
31. The method of any one of aspects 1 to 28, wherein said antigenic protein is selected from the group consisting of: GRP78, HSP60, 60 kDa chaperonin 2, Gelsolin, Chitinase-3-like protein 1, Cathepsin S, Serum albumin, and Cathepsin D, and wherein said autoimmune disease is rheumatoid arthritis (RA).
32. The method of any one of aspects 1 to 28, wherein said antigenic protein is a tumor or cancer antigen such as: an oncogene, proto-oncogene, viral protein, surviving factor or clonotypic or idiotypic determinant, wherein said disease is cancer.
33. A non-immunogenic RNA encoding a peptide, said peptide comprising:
a) an oxidoreductase motif;
b) a T-cell epitope of an antigenic protein; and
c) a linker between a) and b) of between 0 and 7 amino acids, preferably of between 0 and 4 amino acids;
wherein said oxidoreductase motif a) has the following general structure: Zₘ—[CST]-Xₙ—C— or Zₘ—C—Xₙ—[CST], wherein Z is any amino acid, preferably a basic amino acid, more preferably selected from the group comprising: K, H, R and a non-natural basic amino acid, preferably K or H, more preferably K; wherein m is an integer selected from the group comprising: 1, 0, or 2;
wherein X is any amino acid, preferably a basic amino acid, more preferably selected from the group comprising: K, H, R and a non-natural basic amino acid, preferably K or H, more preferably R;
wherein n is an integer selected from 0 to 6, preferably from 0 to 3, most preferably 2, wherein the hyphen (-) in said oxidoreductase motif indicates the point of attachment of the oxidoreductase motif to the N-terminal end of the linker or the T cell epitope, or to the C-terminal end of the linker or the T cell epitope.
34. The non-immunogenic RNA of aspect 33, wherein the non-immunogenic RNA is rendered non-immunogenic by the incorporation of modified nucleotides and the removal of dsRNA.
35. The non-immunogenic RNA of aspect 34, wherein the modified nucleotides suppress RNA-mediated activation of innate immune receptors.
36. The non-immunogenic RNA of aspect 34 or 35, wherein the modified nucleotides comprise a replacement of one or more uridines with a nucleoside comprising a modified nucleobase.
37. The non-immunogenic RNA of aspect 36, wherein the modified nucleobase is a modified uracil.
38. The non-immunogenic RNA of aspect 36 or 37, wherein the nucleoside comprising a modified nucleobase is selected from the group consisting of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine (τm5s2U), 1-taurinomethyl-4-thio-pseudouridine, 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N(1)-methyl-pseudouridine (m1Ψ), 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Urn), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine.
39. The non-immunogenic RNA of any one of aspects 33 to 38, wherein the nucleoside comprising a modified nucleobase is pseudouridine (ψ), N(1)-methyl-pseudouridine (m1ψ) or 5-methyl-uridine (m5U).
40. The non-immunogenic RNA of any one of aspects 33 to 39, wherein the nucleoside comprising a modified nucleobase is N(1)-methyl-pseudouridine (m1ψ).
41. The non-immunogenic RNA of any one of aspects 33 to 40, wherein the non-immunogenic RNA is mRNA.
42. The non-immunogenic RNA of any one of aspects 33 to 41, wherein the non-immunogenic RNA is in vitro transcribed RNA.
43. The non-immunogenic RNA of any one of cl aspects aims 33 to 42, wherein the non-immunogenic RNA is transiently expressed in cells of a subject to whom the pharmaceutical composition is administered.
44. The non-immunogenic RNA of any one of aspects 33 to 43, wherein the non-immunogenic RNA is delivered to dendritic cells of a subject to whom the pharmaceutical composition is administered.
45. The non-immunogenic RNA of aspect 44, wherein the dendritic cells are immature dendritic cells.
46. The non-immunogenic RNA of any one of aspects 33 to 45, wherein the non-immunogenic RNA is formulated in a delivery vehicle.
47. The non-immunogenic RNA of aspect 46, wherein the delivery vehicle comprises particles.
48. The non-immunogenic RNA of aspect 46 or 47, wherein the delivery vehicle comprises a lipid.
49. The non-immunogenic RNA of aspect 48, wherein the lipid comprises a cationic lipid.
50. The non-immunogenic RNA of aspect 48 or 49, wherein the lipid forms a complex with and/or encapsulates the non-immunogenic RNA.
51. The non-immunogenic RNA of any one of aspects 33 to 50, wherein the non-immunogenic RNA is formulated in liposomes.
52. The non-immunogenic RNA of any one of aspects 33 to 51, wherein the oxidoreductase motif in said peptide is selected from the following amino acid motifs:

Zₘ—[CST]-Xₙ—C— or Zₘ—C—Xₙ—[CST]-, (a)
wherein n is 0 (e.g. SEQ ID NO: 733), and wherein m is an integer selected from 0, 1, or 2,
wherein Z is a basic amino acid preferably selected from: H, K, R, and a non-natural basic amino acid as defined herein, such as L-ornithine, more preferably K or H, most preferably K. Non-limiting preferred examples of such motifs are KCC, KKCC (SEQ ID NO: 6), RCC, RRCC (SEQ ID NO: 7), RKCC (SEQ ID NO: 8), or KRCC (SEQ ID NO: 9);

Zₘ—[CST]-Xₙ—C— or Zₘ—C—Xₙ—[CST]-, (b)
wherein n is 1 (e.g. SEQ ID Nos: 734, 750 and 751), wherein X is any amino acid, preferably a basic amino acid selected from: H, K, R, and a non-natural basic amino acid such as L-ornithine, more preferably K or R,
wherein m is an integer selected from 0, 1, or 2,
wherein Z is a basic amino acid preferably selected from: H, K, R, and a non-natural basic amino acid as defined herein, such as L-ornithine, more preferably K or H, most preferably K. Non-limiting preferred examples of such motifs are KCXC (SEQ ID NO: 10), KKCXC (SEQ ID NO: 11), RCXC (SEQ ID NO: 12), RRCXC (SEQ ID NO: 13), RKCXC (SEQ ID NO:14), KRCXC (SEQ ID NO: 15), KCKC (SEQ ID NO: 16), KKCKC (SEQ ID NO: 17), KCRC (SEQ ID NO: 18), KKCRC (SEQ ID NO: 19), RCRC (SEQ ID NO: 20), RRCRC (SEQ ID NO: 21), RKCKC (SEQ ID NO: 22), KRCKC (SEQ ID NO: 23);
(c) Zₘ—[CST]-Xₙ—C— or Zₘ—C—Xₙ—[CST]-, wherein n is 2 (SEQ ID Nos: 723, 728, 735, 740, 745, 752, 757 and 758), thereby creating an internal X¹X² amino acid couple within the oxidoreductase motif, wherein X is any amino acid, preferably wherein at least one X is a basic amino acid selected from: H, K, R, and a non-natural basic amino acid such as L-ornithine, more preferably K or R,
wherein m is an integer selected from 0, 1, or 2,
wherein Z is a basic amino acid preferably selected from: H, K, R, and a non-natural basic amino acid as defined herein, such as L-ornithine, more preferably K or H, most preferably K. these motifs, an internal X¹X² amino acid couple is situated within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2 and Z is a basic amino acid selected from: H, K, or R, or a non-natural basic amino acid as defined herein, such as L-ornithine, preferably K or H. X¹ and X², each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a further example, at least one of X¹ or X² in said motif is a basic amino acid selected from: H, K, or R, or a non-natural basic amino acid as defined herein, such as L-ornithine. In another example of the motif, at least one of X¹ or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Particularly preferred motifs of this form are CPYC (SEQ ID NO: 98), HCPYC, KHCPYC, KCPYC, RCPYC, HCGHC, KCGHC, and RCGHC (corresponding to SEQ ID NOs: 24 to 30). Alternative preferred motifs of this form are KKCPYC, KRCPYC, KHCGHC, KKCGHC, and KRCGHC (SEQ ID NOs: 31 to 35);
(d) Zₘ—[CST]-Xₒ—C— or Zₘ—C—Xₒ—[CST]-, wherein n is 3 (SEQ ID Nos: 724, 729, 736, 741, 746, 753, 759 and 760), thereby creating an internal X¹X²X³ amino acid stretch within the oxidoreductase motif, wherein X is any amino acid, preferably wherein at least one X is a basic amino acid selected from: H, K, R, and a non-natural basic amino acid such as L-ornithine, more preferably K or R,
wherein m is an integer selected from 0, 1, or 2,
wherein Z is a basic amino acid preferably selected from: H, K, R, and a non-natural basic amino acid as defined herein, such as L-ornithine, more preferably K or H.

Preferred are motifs wherein m is 1 or 2 and Z is a basic amino acid selected from: H, K, or R, or a non-natural basic amino acid as defined herein, such as L-ornithine, preferably K or H. In certain examples, X¹, X², and X³, each individually can be any amino acid selected from the group consisting of: G, A, V, L, I, M, F, W, P, S, T, C, Y, N, Q, D, E, K, R, and H, or non-natural amino acids. Preferably, X¹, X², and X³ in said motif is any amino acid except for C, S, or T. In a specific embodiment, at least one of X¹, X², or X³ in said motif is a basic amino acid selected from: H, K, or R, or a non-natural basic amino acid as defined herein, such as L-ornithine. Specific examples of the internal X¹X²X³ amino acid stretch within the oxidoreductase motif are: XPY, PXY, and PYX, wherein X can be any amino acid, preferably a basic amino acid such as K, R, or H, or a non-natural basic amino acid such as L-ornithine. Non-limiting examples include KPY, RPY, HPY, GPY, APY, VPY, LPY, IPY, MPY, FPY, WPY, PPY, SPY, TPY, CPY, YPY, NPY, QPY, DPY, EPY, KPY, PKY, PRY, PHY, PGY, PAY, PVY, PLY, PIY, PMY, PFY, PWY, PPY, PSY, PTY, PCY, PYY, PNY, PQY, PDY, PEY, PLY, PYK, PYR, PYH, PYG, PYA, PYV, PYL, PYI, PYM, PYF, PYW, PYP, PYS, PYT, PYC, PYY, PYN, PYQ, PYD, or PYE. Alternative examples of the internal X¹X²X³ amino acid stretch within the oxidoreductase motif are XHG, HXG, and HGX, wherein X can be any amino acid, such as in KHG, RHG, HHG, GHG, AHG, VHG, LHG, IHG, MHG, FHG, WHG, PHG, SHG, THG, CHG, YHG, NHG, QHG, DHG, EHG, and KHG, HKG, HRG, HHG, HGG, HAG, HVG, HLG, HIG, HMG, HFG, HWG, HPG, HSG, HTG, HCG, HYG, HNG, HQG, HDG, HEG, HLG, HGK, HGR, HGH, HGG, HGA, HGV, HGL, HGI, HGM, HGF, HGW, HGP, HGS, HGT, HGC, HGY, HGN, HGQ, HGD, or HGE. Yet alternative examples of the internal X¹X²X³ amino acid stretch within the oxidoreductase motif are XGP, GXP, and GPX, wherein X can be any amino acid, such as in KGP, RGP, HGP, GGP, AGP, VGP, LGP, IGP, MGP, FGP, WGP, PGP, SGP, TGP, CGP, YGP, NGP, QGP, DGP, EGP, KGP, GKP, GRP, GHP, GGP, GAP, GVP, GLP, GIP, GMP, GFP, GWP, GPP, GSP, GTP, GCP, GYP, GNP, GQP, GDP, GEP, GLP, GPK, GPR, GPH, GPG, GPA, GPV, GPL, GPI, GPM, GPF, GPW, GPP, GPS, GPT, GPC, GPY, GPN, GPQ, GPD, or GPE. Yet alternative examples of the internal X¹X²X³ amino acid stretch within the oxidoreductase motif are XGH, GXH, and GHX, wherein X can be any amino acid, such as in KGH, RGH, HGH, GGH, AGH, VGH, LGH, IGH, MGH, FGH, WGH, PGH, SGH, TGH, CGH, YGH, NGH, QGH, DGH, EGH, KGH, GKH, GRH, GHH, GGH, GAH, GVH, GLH, GIH, GMH, GFH, GWH, GPH, GSH, GTH, GCH, GYH, GNH, GQH, GDH, GEH, GLH, GHK, GHR, GHH, GHG, GHA, GHV, GHL, GHI, GHM, GHF, GHW, GHP, GHS, GHT, GHC, GHY, GHN, GHQ, GHD, or GHE. Yet alternative examples of the internal X¹X²X³ amino acid stretch within the oxidoreductase motif are XGF, GXF, and GFX, wherein X can be any amino acid, such as in KGF, RGF, HGF, GGF, AGF, VGF, LGF, IGF, MGF, FGF, WGF, PGF, SGF, TGF, CGF, YGF, NGF, QGF, DGF, EGF, and KGF, GKF, GRF, GHF, GGF, GAF, GVF, GLF, GIF, GMF, GFF, GWF, GPF, GSF, GTF, GCF, GYF, GNF, GQF, GDF, GEF, GLF, GFK, GFR, GFH, GFG, GFA, GFV, GFL, GFI, GFM, GFF, GFW, GFP, GFS, GFT, GFC, GFY, GFN, GFQ, GFD, or GFE. Yet alternative examples of the internal X¹X²X³ amino acid stretch within the oxidoreductase motif are XRL, RXL, and RLX, wherein X can be any amino acid, such as in KRL, RRL, HRL, GRL, ARL, VRL, LRL, IRL, MRL, FRL, WRL, PRL, SRL, TRL, CRL, YRL, NRL, QRLRL, DRL, ERL, KRL, GKF, GRF, GHF, GGF, GAF, GVF, GLF, GIF, GMF, GFF, GWF, GPF, GSF, GTF, GCF, GYF, GNF, GQF, GDF, GEF, and GLF, RLK, RLR, RLH, RLG, RLA, RLV, RLL, RLI, RLM, RLF, RLW, RLP, RLS, RLT, RLC, RLY, RLN, RLQ, RLD, or RLE. Yet alternative examples of the internal X¹X²X³ amino acid stretch within the oxidoreductase motif are XHP, HXP, and HPX, wherein X can be any amino acid, such as in KHP, RHP, HHP, GHP, AHP, VHP, LHP, IHP, MHP, FHP, WHP, PHP, SHP, THP, CHP, YHP, NHP, QHP, DHP, EHP, KHP, HKP, HRP, HHP, HGP, HAF, HVF, HLF, HIF, HMF, HFF, HWF, HPF, HSF, HTF, HCF, HYP, HNF, HQF, HDF, HEF, HLP, HPK, HPR, HPH, HPG, HPA, HPV, HPL, HPI, HPM, HPF, HPW, HPP, HPS, HPT, HPC, HPY, HPN, HPQ, HPD, or HPE.

Particularly preferred examples are: CRPYC, KCRPYC, KHCRPYC, RCRPYC, HCRPYC, CPRYC, KCPRYC, RCPRYC, HCPRYC, CPYRC, KCPYRC, RCPYRC, HCPYRC, CKPYC, KCKPYC, RCKPYC, HCKPYC, CPKYC, KCPKYC, RCPKYC, HCPKYC, CPYKC, KCPYKC, RCPYKC, and HCPYKC (corresponding to SEQ ID NOs: 36 to 60);
(e) Zₘ—[CST]-Xₙ—C— or Zₘ—C—Xₙ—[CST]-, wherein n is 4 (SEQ ID Nos: 725, 730, 737, 742, 747, 754, 761 and 762), thereby creating an internal X¹X²X³X⁴ (SEQ ID NO: 76) amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K. Preferred are motifs wherein m is 1 or 2 and Z is a basic amino acid selected from: H, K, or R, or a non-natural basic amino acid as defined herein, such as L-ornithine, preferably K or H. X¹, X², X³ and X⁴ each individually can be any amino acid selected from the group consisting of: G, A, V, L, I, M, F, W, P, S, T, C, Y, N, Q, D, E, K, R, and H, or non-natural amino acids as defined herein. Preferably, X¹, X², X³ and X⁴ in said motif is any amino acid except for C, S, or T. In certain non-limiting examples, at least one of X¹, X², X³ or X⁴ in said motif is a basic amino acid selected from: H, K, or R, or a non-natural basic amino acid as defined herein. Specific examples include LAVL (SEQ ID NO: 61), TVQA (SEQ ID NO: 62) or GAVH (SEQ ID NO: 63) and their variants such as: X¹AVL, LX²VL, LAX³L, or LAVX⁴; X¹VQA, TX²QA, TVX³A, or TVQX⁴; X¹AVH, GX²VH, GAX³H, or GAVX⁴ (corresponding to SEQ ID NO: 64 to 75); wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein;
(f) Zₘ—[CST]-Xₙ—C— or Zₘ—C—Xₙ—[CST]-, wherein n is 5 (SEQ ID Nos: 726, 731, 738, 743, 748, 755, 763 and 764), thereby creating an internal X¹X²X³X⁴X⁵ (SEQ ID NO: 77) amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K. Preferred are motifs wherein m is 1 or 2 and Z is a basic amino acid selected from: H, K, or R, or a non-natural basic amino acid as defined herein, such as L-ornithine, preferably K or H. X¹, X², X³, X⁴ and X⁵ each individually can be any amino acid selected from the group consisting of: G, A, V, L, I, M, F, W, P, S, T, C, Y, N, Q, D, E, K, R, and H, or non-natural amino acids. Preferably, X¹, X², X³, X⁴ and X⁵ in said motif is any amino acid except for C, S, or T. In certain examples, at least one of X¹, X², X³ X⁴ or X⁵ in said motif is a basic amino acid selected from: H, K, or R, or a non-natural basic amino acid as defined herein. Specific examples include PAFPL (SEQ ID NO: 78) or DQGGE (SEQ ID NO: 79) and their variants such as: X¹AFPL, PX²FPL, PAX³PL, PAFX⁴L, or PAFPX⁵; X¹QGGE, DX²GGE, DQX³GE, DQGX⁴E, or DQGGX⁵ (corresponding to SEQ ID NO: 80 to 89), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural amino acids as defined herein;
(g) Zₘ—[CST]-Xₙ—C— or Zₘ—C—Xₙ—[CST]-, wherein n is 6 (SEQ ID Nos: 727, 732, 739, 744, 749, 756, 765 and 766), thereby creating an internal X¹X²X³X⁴X⁵X⁶ (SEQ ID NO: 90) amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K. Preferred are motifs wherein m is 1 or 2 and Z is a basic amino acid selected from: H, K, or R, or a non-natural basic amino acid as defined herein, such as L-ornithine, preferably K or H. X¹, X², X³, X⁴ X⁵ and X⁶ each individually can be any amino acid selected from the group consisting of: G, A, V, L, I, M, F, W, P, S, T, C, Y, N, Q, D, E, K, R, and H, or non-natural amino acid. Preferably, X¹, X², X³, X⁴, X⁵ and X⁶ in said motif is any amino acid except for C, S, or T. In certain examples, at least one of X¹, X², X³ X⁴, X⁵ or X⁶ in said motif is a basic amino acid selected from: H, K, or R, or a non-natural basic amino acid as defined herein. Specific examples include DIADKY (SEQ ID NO: 91) or variants thereof such as: X¹IADKY, DX²ADKY, DIX³DKY, DIAX⁴KY, DIADX⁵Y, or DIADKX⁶ (corresponding to SEQ ID NO: 92 to 97), wherein X¹, X², X³, X⁴, X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein; or
(h) Zₘ—[CST]-Xₙ—C— or Zₘ—C—Xₙ—[CST]-, wherein n is 0 to 6 and wherein m is 0, and wherein one of the C or [CST] residues has been modified so as to carry an acetyl, methyl, ethyl or propionyl group, either on the N-terminal amide (SEQ ID Nos: 770-779) of the amino acid residue of the motif or on the C-terminal carboxy group (SEQ ID Nos: 780-789). In preferred embodiments of such a motif, n is 2 and m is 0, wherein the internal X¹X², each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a further example, at least one of X¹ or X² in said motif is a basic amino acid selected from: H, K, or R, or a non-natural basic amino acid as defined herein, such as L-ornithine. In another example of the motif, at least one of X¹ or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Preferably said modification results in an N-terminal acetylation of the first cysteine in the motif (N-acetyl-cysteine).
53. The non-immunogenic RNA of any one of aspects 33 to 52, wherein the Xₙ portion of the oxidoreductase motif in said peptide comprises the sequence PY, preferably wherein the oxidoreductase motif comprises the sequence CPYC (SEQ ID NO: 98), or has any one of the following sequences: HCPYC, KHCPYC, KCPYC, RCPYC, HCGHC, KCGHC, RCGHC, KKCPYC, KRCPYC, KHCGHC, KKCGHC, and KRCGHC (SEQ ID NOs: 24 to 35).
54. The non-immunogenic RNA of any one of aspects 33 to 53, wherein amino acid Z of the oxidoreductase motif in said peptide is a basic amino acid selected from the group of amino acids consisting of: H, K, R, and any non-natural basic amino acid, more preferably a basic amino acid selected from: H, K, and R, most preferably wherein Z is H or K.
55. The non-immunogenic RNA of any one of aspects 33 to 54, wherein said immunogenic peptide has a length of between 9 and 50 amino acids, preferably of between 9 and 30 amino acids.
56. The non-immunogenic RNA of any one of aspects 33 to 55, wherein the oxidoreductase motif in said peptide does not naturally occur in the amino acid sequence within a region of 11 amino acids N-terminally or C-terminally of the T-cell epitope in said antigenic protein and/or wherein said the T-cell epitope does not naturally comprise said oxidoreductase motif in its amino acid sequence.
57. The non-immunogenic RNA of any one of aspects 33 to 56, wherein said antigenic protein is selected from the group consisting of: (pro)insulin, GAD65, GAD67, IA-2 (ICA512), IA-2 (beta/phogrin), IGRP, Chromogranin, ZnT8 and HSP-60, and wherein said autoimmune disease is type-1 diabetes (T1D).
58. The non-immunogenic RNA of any one of aspects 33 to 56, wherein said antigenic protein is selected from the group consisting of: Myelin oligodendrocyte glycoprotein (MOG), Myelin basic protein (MBP), Proteolipid protein (PLP), Myelin-oligodendrocytic basic protein (MOBP), and Oligodendrocyte-specific protein (OSP), and wherein said autoimmune disease is multiple sclerosis (MS), and/or neuromyelitis optica (NMO).
59. The non-immunogenic RNA of any one of aspects 33 to 56, wherein said antigenic protein is selected from the group consisting of: GRP78, HSP60, 60 kDa chaperonin 2, Gelsolin, Chitinase-3-like protein 1, Cathepsin S, Serum albumin, and Cathepsin D, and wherein said autoimmune disease is rheumatoid arthritis (RA).
60. The non-immunogenic RNA of any one of aspects 33 to 56, wherein said antigenic protein is Myelin oligodendrocyte glycoprotein (MOG), wherein said autoimmune disease is neuromyelitis optica (NMO).
61. The non-immunogenic RNA of any one of aspects 33 to 60 for use in the method of any one of claims 1 to 32.
62. A pharmaceutical composition comprising the non-immunogenic RNA according to any one of aspects 33 to 60 and optionally a pharmaceutically acceptable excipient.
63. In a preferred embodiment of any one of aspects 1 to 62, the linker in the peptide comprises at least 1 amino acid, at least 2 amino acids, at least 3 amino acids, or at least 4 amino acids. Preferably, said linker comprises between 1 and 7 amino acids, such as between 2 and 7 amino acids, between 3 and 7 amino acids, or between 4 and 7 amino acids.
64. In another preferred embodiment of any one of aspects 1 to 63, the T-cell epitope of the peptide does not comprise a basic amino acid at its N-terminal end, i.e. immediately adjacent to the linker or oxidoreductase motif, more particularly in case the linker is absent or only comprises 1 or 2 amino acids. More preferably, in all aspects, the T-cell epitope does not comprise a basic amino acid at its N-terminal end, i.e. immediately adjacent to the linker or oxidoreductase motif, more particularly in case the linker is absent or only comprises 1 or 2 amino acids.
65. In a further embodiment of any one of aspects 1 to 64, the T-cell epitope of the peptide does not comprise a basic amino acid in position 1, 2 and/or 3 counted from its N-terminal end, i.e. immediately adjacent to the linker or oxidoreductase motif, more particularly in case the linker is absent or only comprises 1 or 2 amino acids.
66. In any one of aspects 1 to 65, the oxidoreductase motif in the peptide forms the N-terminal end of said peptide. In an alternative set of embodiments, the oxidoreductase motif forms the C-terminal end of the peptide.
67. In any one of aspects 1 to 66, patients being treated for MS typically have HLA HLA-DRB1* types selected from the group consisting of: HLA-DRB1*15:01, HLA-DRB1*03:01, HLA-DRB1*04:01, and HLA-DRB1*07:01, preferably HLA-DRB1*15:01.
68. In any one of aspects 1 to 66, patients being treated for NMO typically have HLA types selected from the group consisting of: HLA-DRB1*03:01 and HLA-DPB1*05:01 (for Asia).
69. In any one of aspects 1 to 66, patients being treated for T1D, typically have HLA types selected from the group consisting of: HLA-DRB1*03:01 and 04:01.
70. In any one of aspects 1 to 66, patients being treated for RA typically have HLA types selected from the group consisting of: HLA-DRB1*01:01, 04:01, and 04:04.
71. In a preferred embodiment of any one of aspects 1 to 70, said T cell epitope in the peptide is an NKT cell epitope having a length of between 7 and 25 amino acids; or said T cell epitope is an MHC class II T cell epitope having a length of between 7 and 25 amino acids, preferably between 9 and 25 amino acids.
72. In a preferred embodiment of any one of aspects 1 to 71, said T cell epitope in the peptide is an NKT cell epitope having a length of between 7 and 50 amino acids, or said peptide comprises an MHC class II T cell epitope has a length of between 7 and 50 amino acids, preferably between 9 and 50 amino acids.
73. In any one of the aspects listed herein, the oxidoreductase motif is not part of a repeat of the standard C—XX—[CST] or [CST]-XX—C oxidoreductase motifs such as repeats of said motif which can be spaced from each other by one or more amino acids (e.g. CXXC X CXXC X CXXC (SEQ ID NO: 99)), as repeats which are adjacent to each other (CXXCCXXCCXXC (SEQ ID NO: 100)) or as repeats which overlap with each other CXXCXXCXXC (SEQ ID NO: 101) or CXCCXCCXCC (SEQ ID NO: 102)), especially when n is 0 or 1 and m is 0.

### BRIEF DESCRIPTION OF THE DRAWINGS

FIG. 1: represents blinded evaluation of clinical EAE scoring (0-5) performed daily from day 7 to day 28. Mice were injected with MOG₃₅₋₅₅ to induce EAE at day 0 and were left untreated (vehicle) or therapeutically treated with IMCY-0189 m1Ψ LNP-encapsulated mRNA or MOG₃₅₋₅₅ m1Ψ LNP-encapsulated mRNA (see table 2 for details). The mean clinical score was determined each day for each group of mice.

FIG. 2: represents AUC calculated from EAE scores displayed in FIG. 1 for each group of mice. Significant differences are referred as follows: *p<0.05, **p<0.01.

FIG. 3: represents MMS calculated from EAE scores displayed in FIG. 1 for each group of mice. Significant difference is referred as follows: *p<0.05.

### DETAILED DESCRIPTION OF THE INVENTION

The present invention will be described with respect to particular embodiments, although the invention is not limited thereto but only by the claims. Any reference signs in the claims shall not be construed as limiting the scope. The following terms or definitions are provided solely to aid in the understanding of the invention. Unless specifically defined herein, all terms used herein have the same meaning as they would have to one skilled in the art of the present invention. The definitions provided herein should not be construed to have a scope less than the one understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise. The term “any” when used in relation to aspects, claims or embodiments as used herein refers to any single one (i.e. anyone) as well as to all combinations of said aspects, claims or embodiments referred to.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. Said terms also encompass the embodiments “consisting essentially of” and “consisting of”.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

As used herein, the term “for use” as used in “composition for use in treatment of a disease” shall disclose also the corresponding method of treatment and the corresponding use of a preparation for the manufacture of a medicament for the treatment of a disease”.

The term “peptide” as used herein refers to a molecule comprising an amino acid sequence of between 9 and 200 amino acids, connected by peptide bonds, but which can comprise non-amino acid structures.

The term “immunogenic peptide” as used herein refers to a peptide that is immunogenic, i.e. that comprises a T-cell epitope capable of eliciting an immune response.

Peptides according to the invention can contain any of the conventional 20 amino acids or modified versions thereof, or can contain non-naturally occurring amino-acids incorporated by chemical peptide synthesis or by chemical or enzymatic modification.

The term “antigen” as used herein refers to a structure of a macromolecule, typically a protein (with or without polysaccharides) or made of proteic composition comprising one or more hapten(s) and comprising T or NKT cell epitopes.

The term “antigenic protein” as used herein refers to a protein comprising one or more T or NKT cell epitopes. An auto-antigen or auto-antigenic protein as used herein refers to a human or animal protein or fragment thereof present in the body, which elicits an immune response within the same human or animal body.

The term “food or pharmaceutical antigenic protein” refers to an antigenic protein present in a food or pharmaceutical product, such as in a vaccine.

The term “epitope” refers to one or several portions (which may define a conformational epitope) of an antigenic protein which is/are specifically recognised and bound by an antibody or a portion thereof (Fab′, Fab2′, etc.) or a receptor presented at the cell surface of a B—, or T-, or NKT cell, and which is able, by said binding, to induce an immune response.

The term “T cell epitope” in the context of the present invention refers to a dominant, sub-dominant or minor T cell epitope, i.e. a part of an antigenic protein that is specifically recognised and bound by a receptor at the cell surface of a T lymphocyte. Whether an epitope is dominant, sub-dominant or minor depends on the immune reaction elicited against the epitope. Dominance depends on the frequency at which such epitopes are recognised by T cells and able to activate them, among all the possible T cell epitopes of a protein. The T cell epitope can be an epitope recognised by MHC class II molecules or an NKT cell epitope recognised by a CD1d molecule.

A T cell epitope can be an epitope recognised by MHC class II molecules, typically consists of a sequence of 9 amino acids that fits in the groove of the MHC II molecule. Within a peptide sequence representing an MHC class II T cell epitope, the amino acids in the epitope can be numbered P1 to P9, amino acids N-terminal of the epitope are numbered P−1, P−2 and so on, amino acids C terminal of the epitope are numbered P+1, P+2 and so on. Peptides recognised by MHC class II molecules and not by MHC class I molecules are referred to as MHC class II restricted T cell epitopes.

The identification and selection of a T-cell epitope from antigenic proteins is known to a person skilled in the art.

To identify an epitope suitable in the context of the present invention, isolated peptide sequences of an antigenic protein are tested by, for example, T cell biology techniques, to determine whether the peptide sequences elicit a T cell response. Those peptide sequences found to elicit a T cell response are defined as having T cell stimulating activity.

Human T cell stimulating activity can further be tested by culturing T cells obtained from e.g. an individual having T1D, with a peptide/epitope derived from the auto-antigen involved in T1D and determining whether proliferation of T cells occurs in response to the peptide/epitope as measured, e.g., by cellular uptake of tritiated thymidine. Stimulation indices for responses by T cells to peptides/epitopes can be calculated as the maximum CPM in response to a peptide/epitope divided by the control CPM. A T cell stimulation index (S.I.) equal to or greater than two times the background level is considered “positive.” Positive results are used to calculate the mean stimulation index for each peptide/epitope for the group of peptides/epitopes tested.

Non-natural (or modified) T-cell epitopes can further optionally be tested on their binding affinity to MHC class II molecules. This can be performed in different ways. For instance, soluble HLA class II molecules are obtained by lysis of cells homozygous for a given class II molecule. The latter is purified by affinity chromatography. Soluble class II molecules are incubated with a biotin-labelled reference peptide produced according to its strong binding affinity for that class II molecule. Peptides to be assessed for class II binding are then incubated at different concentrations and their capacity to displace the reference peptide from its class II binding is calculated by addition of neutravidin.

In order to determine optimal T cell epitopes by, for example, fine mapping techniques, a peptide having T cell stimulating activity and thus comprising at least one T cell epitope as determined by T cell biology techniques is modified by addition or deletion of amino acid residues at either the amino- or carboxy-terminus of the peptide and tested to determine a change in T cell reactivity to the modified peptide. If two or more peptides which share an area of overlap in the native protein sequence are found to have human T cell stimulating activity, as determined by T cell biology techniques, additional peptides can be produced comprising all or a portion of such peptides and these additional peptides can be tested by a similar procedure. Following this technique, peptides are selected and produced recombinantly or synthetically. T cell epitopes or peptides are selected based on various factors, including the strength of the T cell response to the peptide/epitope (e.g., stimulation index) and the frequency of the T cell response to the peptide in a population of individuals.

Additionally and/or alternatively, one or more in vitro algorithms can be used to identify a T cell epitope sequence within an antigenic protein. Suitable algorithms include, but are not limited to those described in Zhang et al. (2005) Nucleic Acids Res 33, W180-W183 (PREDBALB); Salomon & Flower (2006) BMC Bioinformatics 7, 501 (MHCBN); Schuler et al. (2007) Methods Mol. Biol.409, 75-93 (SYFPEITHI); Donnes & Kohlbacher (2006) Nucleic Acids Res. 34, W194-W197 (SVMHC); Kolaskar & Tongaonkar (1990) FEBS Lett. 276, 172-174, Guan et al. (2003) Appl. Bioinformatics 2, 63-66 (MHCPred) and Singh and Raghava (2001) Bioinformatics 17, 1236-1237 (Propred). More particularly, such algorithms allow the prediction within an antigenic protein of one or more octa- or nonapeptide sequences which will fit into the groove of an MHC II molecule and this for different HLA types.

The term “MHC” refers to “major histocompatibility antigen”. In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such the terms “MHC” and “HLA” are equivalents when used herein. The HLA system in man has its equivalent in the mouse, i.e., the H2 system. The most intensely-studied HLA genes are the nine so-called classical MHC genes: HLA-A, HLA-B, HLA-C, HLA-DPA1, HLA-DPB1, HLA-DQA1, HLAs DQB1, HLA-DRA, and HLA-DRB1. In humans, the MHC is divided into three regions: Class I, II, and III. The A, B, and C genes belong to MHC class I, whereas the six D genes belong to class II. MHC class I molecules are made of a single polymorphic chain containing 3 domains (alpha 1, 2 and 3), which associates with beta 2 microglobulin at cell surface. Class II molecules are made of 2 polymorphic chains, each containing 2 chains (alpha 1 and 2, and beta 1 and 2).

Patients being treated for MS typically have HLA HLA-DRB1*types selected from the group consisting of: HLA-DRB1*15:01, HLA-DRB1*03:01, HLA-DRB1*04:01, and HLA-DRB1*07:01, preferably HLA-DRB1*15:01.

Patients being treated for NMO typically have HLA types selected from the group consisting of: HLA-DRB1*03:01 and HLA-DPB1*05:01 (for Asia).

Patients being treated for T1D, typically have HLA types selected from the group consisting of: HLA-DRB1*03:01 and 04:01.

Patients being treated for RA typically have HLA types selected from the group consisting of: HLA-DRB1*01:01, 04:01, and 04:04.

Class I MHC molecules are expressed on virtually all nucleated cells.

Peptide fragments presented in the context of class I MHC molecules are recognised by CD8+T lymphocytes (cytolytic T lymphocytes or CTLs). CD8+T lymphocytes frequently mature into cytolytic effectors which can lyse cells bearing the stimulating antigen. Class II MHC molecules are expressed primarily on activated lymphocytes and antigen-presenting cells. CD4+T lymphocytes (helper T lymphocytes or Th) are activated with recognition of a unique peptide fragment presented by a class II MHC molecule, usually found on an antigen-presenting cell like a macrophage or dendritic cell. CD4+T lymphocytes proliferate and secrete cytokines such as IL-2, IFN-gamma and IL-4 that support antibody-mediated and cell mediated responses.

Functional HLAs are characterised by a deep binding groove to which endogenous as well as foreign, potentially antigenic peptides bind. The groove is further characterised by a well-defined shape and physico-chemical properties. HLA class I binding sites are closed, in that the peptide termini are pinned down into the ends of the groove. They are also involved in a network of hydrogen bonds with conserved HLA residues. In view of these restraints, the length of bound peptides is limited to 8, 9 or 10 residues. However, it has been demonstrated that peptides of up to 12 amino acid residues are also capable of binding HLA class I. Comparison of the structures of different HLA complexes confirmed a general mode of binding wherein peptides adopt a relatively linear, extended conformation, or can involve central residues to bulge out of the groove.

In contrast to HLA class I binding sites, class II sites are open at both ends. This allows peptides to extend from the actual region of binding, thereby “hanging out” at both ends. Class II HLAs can therefore bind peptide ligands of variable length, ranging from 9 to more than 25 amino acid residues. Similar to HLA class I, the affinity of a class II ligand is determined by a “constant” and a “variable” component. The constant part again results from a network of hydrogen bonds formed between conserved residues in the HLA class II groove and the main-chain of a bound peptide. However, this hydrogen bond pattern is not confined to the N- and C-terminal residues of the peptide but distributed over the whole chain. The latter is important because it restricts the conformation of complexed peptides to a strictly linear mode of binding. This is common for all class II allotypes. The second component determining the binding affinity of a peptide is variable due to certain positions of polymorphism within class II binding sites. Different allotypes form different complementary pockets within the groove, thereby accounting for subtype-dependent selection of peptides, or specificity. Importantly, the constraints on the amino acid residues held within class II pockets are in general “softer” than for class I. There is much more cross reactivity of peptides among different HLA class II allotypes. The sequence of the +/−9 amino acids (i.e. 8, 9 or 10) of an MHC class II T cell epitope that fit in the groove of the MHC II molecule are usually numbered P1 to P9. Additional amino acids N-terminal of the epitope are numbered P−1, P−2 and so on, amino acids C-terminal of the epitope are numbered P+1, P+2 and so on.

The term “NKT cell epitope” refers to a part of an antigenic protein that is specifically recognized and bound by a receptor at the cell surface of an NKT cell and typically has a length of 7 amino acids. In particular, an NKT cell epitope is an epitope bound by CD1d molecules. The NKT cell epitope has a general motif [FWYHT]—X(2)-[VILM]-X(2)-[FWYHT](SEQ ID NO: 103). Alternative versions of this general motif have at position 1 and/or position 7 the alternatives [FWYH], thus [FWYH]—X(2)-[VILM]-X(2)-[FWYH](SEQ ID NO: 104).

Alternative versions of this general motif have at position 1 and/or position 7 the alternatives [FWYT], [FWYT]-X(2)-[VILM]-X(2)-[FWYT](SEQ ID NO: 105). Alternative versions of this general motif have at position 1 and/or position 7 the alternatives [FWY], [FWY]—X(2)-[VILM]-X(2)-[FWY](SEQ ID NO: 106).

Regardless of the amino acids at position 1 and/or 7, alternative versions of the general motif have at position 4 the alternatives [ILM], e.g. [FWYH]—X(2)-[ILM]-X(2)-[FWYH](SEQ ID NO: 107) or [FWYHT]-X(2)-[ILM]-X(2)-[FWYHT](SEQ ID NO: 108) or [FWY]—X(2)-[ILM]-X(2)-[FWY](SEQ ID NO: 109). Such hydrophobic peptides are characterized by a motif in which positions P1 and P7 are occupied by hydrophobic residues such as phenylalanine (F) or tryptophan (W). P7 is however permissive in the sense that it accepts alternative hydrophobic residues to phenylalanine or tryptophan, such as threonine (T) or histidine (H). The P4 position is occupied by an aliphatic residue such as isoleucine (I), leucine (L) or methionine (M). such peptides may have hydrophobic residues which naturally constitute a CD1d binding motif. In some embodiment, amino acid residues of said motif are modified, usually by substitution with residues which increase the capacity to bind to CD1d. In a specific embodiment motifs are modified to fit more closely with said general motif. More particularly, peptides are produced to contain a F or W at position 7.

A CD1d binding motif in a protein can be identified by scanning a sequence for the above sequence motifs, either by hand, either by using an algorithm such as ScanProsite De Castro E. et al. (2006) Nucleic Acids Res. 34(Web Server issue):W362-W365.

“Natural killer T” or “NKT” cells constitute a distinct subset of non-conventional T lymphocytes that recognize antigens presented by the non-classical MHC complex molecule CD1d. Two subsets of NKT cells are presently described. Type I NKT cells, also called invariant NKT cells (iNKT), are the most abundant. They are characterized by the presence of an alpha-beta T cell receptor (TCR) made of an invariant alpha chain, Valphal4 in the mouse and Valpha24 in humans. This alpha chain is associated to a variable though limited number of beta chains. Type 2 NKT cells have an alpha-beta TCR but with a polymorphic alpha chain. However, it is apparent that other subsets of NKT cells exist, the phenotype of which is still incompletely defined, but which share the characteristics of being activated by glycolipids presented in the context of the CD1d molecule.

NKT cells typically express a combination of natural killer (NK) cell receptor, including NKG2D and NK1.1. NKT cells are part of the innate immune system, which can be distinguished from the adaptive immune system by the fact that they do not require expansion before acquiring full effector capacity. Most of their mediators are preformed and do not require transcription. NKT cells have been shown to be major participants in the immune response against intracellular pathogens and tumor rejection. Their role in the control of autoimmune diseases and of transplantation rejection is also advocated.

The recognition unit, the CD1d molecule, has a structure closely resembling that of the MHC class I molecule, including the presence of beta-2 microglobulin. It is characterized by a deep cleft bordered by two alpha chains and containing highly hydrophobic residues, which accepts lipid chains. The cleft is open at both extremities, allowing it to accommodate longer chains. The canonical ligand for CD1d is the synthetic alpha galactosylceramide (alpha GalCer). However, many natural alternative ligands have been described, including glyco- and phospholipids, the natural lipid sulfatide found in myelin, microbial phosphoinositol mannoside and alpha-glucuronosylceramide. The present consensus in the art (Matsuda et al (2008), Curr. Opinion Immunol., 20 358-368; Godfrey et al (2010), Nature rev. Immunol 11, 197-206) is still that CD1d binds only ligands containing lipid chains, or in general a common structure made of a lipid tail which is buried into CD1d and a sugar residue head group that protrudes out of CD1d.

The term “homologue” as used herein with reference to the epitopes used in the context of the invention, refers to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular homologues of an epitope correspond to the natural epitope modified in at most three, more particularly in at most 2, most particularly in one amino acid.

The term “derivative” as used herein with reference to the peptides of the invention refers to molecules which contain at least the peptide active portion (i.e. the oxidoreductase motif and the MHC class II epitope capable of eliciting cytolytic CD4+ T cell activity) and, in addition thereto comprises a complementary portion which can have different purposes such as stabilising the peptides or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. In particular, the sequence identity is from 70% to 80%, from 81% to 85%, from 86% to 90%, from 91% to 95%, from 96% to 100%, or 100%.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. The nucleic acid encoding a peptide according to the invention or fragment thereof is a sequence encoding the peptide or fragment thereof originating from a mammal or corresponding to a mammalian, most particularly a human peptide fragment.

The term “oxidoreductase motif”, “thiol-oxidoreductase motif”, “thioreductase motif”, “thioredox motif” or “redox motif” are used herein as synonymous terms and refers to motifs involved in the transfer of electrons from one molecule (the reductant, also called the hydrogen or electron donor) to another (the oxidant, also called the hydrogen or electron acceptor). In particular, the term “oxidoreductase motif” can refer to the known [CST]XXC (SEQ ID NO: 110) or CXX[CST](SEQ ID NO: 111) motifs, but particularly refers to the sequence motif [CST]XₙC (SEQ ID NO: 112) or CXₙ[CST](SEQ ID NO: 113), wherein n is an integer selected from the group comprising: 0, 1, 3, 4, 5 or 6, and in which C stands for cysteine, S for serine, T for threonine and X for any amino acid. In order to have reducing activity, the cysteines present in a modified oxidoreductase motif should not occur as part of a cystine disulfide bridge. More particularly, said oxidoreductase motif has the following general structure: Zm-[CST]-Xn-C— or Zm-C-Xn-[CST]-, wherein Z is any amino acid, preferably a basic amino acid, more preferably selected from the group comprising: K, H, R and a non-natural basic amino acid, preferably K or H, more preferably K; wherein m is an integer selected from the group comprising: 1, 0, or 2;
wherein X is any amino acid, preferably a basic amino acid, more preferably selected from the group comprising: K, H, R and a non-natural basic amino acid, preferably K or H, more preferably R;
wherein n is an integer selected from 0 to 6, preferably 0 to 3, most preferably 2. In a preferred embodiment, said motif comprises [CST]-XX—C—(SEQ ID NO: 1) or C—XX—[CST]-(SEQ ID NO: 2), wherein X is any amino acid.

The term “basic amino acid” refers to any amino acid that acts like a Bronsted-Lowry and Lewis base, and includes natural basic amino acids such as Arginine (R), Lysine (K) or Histidine (H), or non-natural basic amino acids, such as, but not limited to:
lysine variants like Fmoc-β-Lys(Boc)-OH (CAS Number 219967-68-7), Fmoc-Orn(Boc)-OH also called L-ornithine or ornithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)—OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

The immunogenic peptides disclosed herein typically can be of use in treating diseases caused by an elevated or uncontrolled immune response towards an allergen or (auto)antigen. Typically said antigenic protein is an auto-antigen, a soluble allofactor, an alloantigen shed by the graft, an antigen of an intracellular pathogen, an antigen of a viral vector used for gene therapy or gene vaccination, a tumor-associated antigen or an allergen. More preferably, said antigenic protein is an autoantigen involved in type-1 diabetes (T1D), a demyelinating disorder such as multiple sclerosis (MS) or neuromyelitis optica (NMO), or rheumatoid arthritis (RA).

The term “immune disorders” or “immune diseases” refers to diseases wherein a reaction of the immune system is responsible for or sustains a malfunction or non-physiological situation in an organism. Included in immune disorders are, inter alia, allergic disorders and autoimmune diseases.

The terms “allergic diseases” or “allergic disorders” as used herein refer to diseases characterised by hypersensitivity reactions of the immune system to specific substances called allergens (such as pollen, stings, drugs, or food). Allergy is the ensemble of signs and symptoms observed whenever an atopic individual patient encounters an allergen to which he has been sensitised, which may result in the development of various diseases, in particular respiratory diseases and symptoms such as bronchial asthma. Various types of classifications exist and mostly allergic disorders have different names depending upon where in the mammalian body it occurs. “Hypersensitivity” is an undesirable (damaging, discomfort-producing and sometimes fatal) reaction produced in an individual upon exposure to an antigen to which it has become sensitised; “immediate hypersensitivity” depends on the production of IgE antibodies and is therefore equivalent to allergy.

The terms “autoimmune disease” or “autoimmune disorder” refer to diseases that result from an aberrant immune response of an organism against its own cells and tissues due to a failure of the organism to recognise its own constituent parts (down to the sub-molecular level) as “self”. The group of diseases can be divided in two categories, organ-specific and systemic diseases.

An “allergen” is defined as a substance, usually a macromolecule or a proteic composition which elicits the production of IgE antibodies in predisposed, particularly genetically disposed, individuals (atopics) patients. Similar definitions are presented in Liebers et al. (1996) Clin. Exp. Allergy 26, 494-516.

The term “demyelination” as used herein refers to damaging and/or degradation of myelin sheaths that surround axons of neurons which has as a consequence the formation of lesions or plaques. It is understood that the myelin acts as a protective covering surrounding nerve fibers in brain, optic nerves, and spinal cord. Due to demyelination, the signal conduction along the affected nerves is impaired (i.e. slowed or stopped), and may cause neurological symptoms such as deficiencies in sensation, movement, cognition, and/or other neurological function. The concrete symptoms a patient suffering from a demyelinating disease will vary depending on the disease and disease progression state. These may include a blurred and/or double vision, ataxia, clonus, dysarthria, fatigue, clumsiness, hand paralysis, hemiparesis, genital anaesthesia, incoordination, paresthesias/paraesthesia, ocular paralysis, impaired muscle coordination, muscle weakness, loss of sensation, impaired vision, neurological symptoms, unsteady way of walking (gait), spastic paraparesis, incontinence, hearing problems, speech problems, and others.

Therefore, “demyelinating diseases” or “demyelinating disorders” as used herein and commonly used in the art is indicative for any pathologic condition of the nervous system which involves impairment, for example damaging, or the myelin sheath of neurons. Demyelinating diseases may be stratified into central nervous system demyelinating diseases and peripheral nervous system. Alternatively, demyelinating diseases may be classified according to the cause of demyelination: destruction of myelin (demyelinating myelinoclastic), or abnormal and degenerative myelin (dysmyelinating leukodystrophic). Non-limiting examples of demyelinating diseases are Multiple Sclerosis (MS—(e.g. Relapsing/Remitting Multiple Sclerosis, Secondary Progressive Multiple Sclerosis, Progressive Relapsing Multiple Sclerosis, Primary Progressive Multiple Sclerosis, and Acute Fulminant Multiple Sclerosis), Neuromyelitis Optica (NMO), Optic Neuritis, Acute Disseminated Encephalomyelitis, Balo's Disease, HTLV-I Associated Myelopathy, Schilder's Disease, Transverse Myelitis, Idiopathic inflammatory demyelinating diseases, vitamin B12-induced central nervous system neuropathies, Central pontine myelinolysis, Myelopathies including tabes dorsalis, Leukodystrophies such as Adrenoleukodystrophy, Leukoencephalopathies such as Progressive multifocal leukoencephalopathy (PML), and Rubella induced mental retardation. It is appreciated by a skilled person that several of the above-mentioned annotations are general classification names indicative of a group of diseases characterized be an identical or similar set of aberrant processes at the molecular level and/or an identical or similar set of (clinical) symptoms. A human patient having a demyelinating disorder can have one or more symptoms of a demyelinating disorder such as, but not limited to, impaired vision, numbness, weakness in extremities, tremors or spasticity, heat intolerance, speech impairment, incontinence, dizziness, or impaired proprioception (e.g., balance, coordination, sense of limb position). A human (e.g., a human patient) with a family history of a demyelinating disorder (e.g., a genetic predisposition for a demyelinating disorder), or who exhibits mild or infrequent symptoms of a demyelinating disorder described above can be, for the purposes of the method, considered at risk of developing a demyelinating disorder (e.g., Multiple Sclerosis). Preferred demyelinating diseases in the context of the current disclosure are those caused by MOG autoantigens or involving anti-MOG antibodies, including but not limited to Multiple Sclerosis (MS) or Neuromyelitis Optica (NMO).

The term “Multiple Sclerosis”, abbreviated herein and in the art as “MS”, indicates an autoimmune disorder affecting the central nervous system. MS is considered the most common non-traumatic disabling disease in young adults (Dobson and Giovannoni, (2019) Eur. J. Neurol. 26(1), 27-40), and the most common autoimmune disorder affecting the central nervous system (Berer and Krishnamoorthy (2014) FEBS Lett. 588(22), 4207-4213). MS may manifest itself in a subject by a large number of different symptoms ranging from physical over mental to psychiatric problems. Typical symptoms include blurred or double vision, muscle weakness, blindness in one eye, and difficulties in coordination and sensation. In most cases, MS may be viewed as a two-stage disease, with early inflammation responsible for relapsing-remitting disease and delayed neurodegeneration causing non-relapsing progression, i.e. secondary and primary progressive MS. Although progress is being made in the field, a conclusive underlying cause of the disease remains hitherto elusive and over 150 single nucleotide polymorphisms have been associated with MS susceptibility (International Multiple Sclerosis Genetics Consortium Nat Genet. (2013). 45(11):1353-60). Vitamin D deficiency, smoking, ultraviolet B (UVB) exposure, childhood obesity and infection by Epstein-Barr virus have been reported to contribute to disease development (Ascherio (2013) Expert Rev Neurother. 13(12 Suppl), 3-9).

Hence, MS can be regarded as a single disease existing within a spectrum extending from relapsing (wherein inflammation is the dominant feature) to progressive (neurodegeneration dominant). Therefore, it is evident that the term Multiple sclerosis as used herein encompasses any type of Multiple Sclerosis belonging to any kind of disease course classification. In particular the invention is envisaged to be a potent treatment strategy patient diagnosed with, or suspected of having clinically Isolated Syndrome (CIS), relapse-remitting MS (RRMS), secondary progressive MS (SPMS), primary progressive MS (PPMS), and even MS-suspected radiology isolated syndrome (RIS). While strictly not considered a disease course of MS, RIS is used to classify subjects showing abnormalities on the Magnetic Resonance Imaging (MRI) of brain and/or spinal cord that correspond to MS lesions and cannot be prima facie explained by other diagnoses. CIS is a first episode (by definition lasting for over 24 hours) of neurologic symptoms caused by inflammation and demyelination in the central nervous system. In accordance with RIS, CIS classified subjects may or may not continue to develop MS, with subjects showing MS-like lesions on a brain MRI more likely to develop MS. RRMS is the most common disease course of MS with 85% of subjects having MS being diagnosed with RRMS. RRMS diagnosed patients are a preferred group of patients in view of the current invention. RRMS is characterized by attacks of new or increasing neurologic symptoms, alternatively worded relapses or exacerbations. In RRMS, said relapses are followed by periods or partial or complete remission of the symptoms, and no disease progression is experienced and/or observed in these periods of remission. RRMS may be further classified as active RRMS (relapses and/or evidence of new MRI activity), non-active RRMS, worsening RRMS (increasing disability over a specified period of time after a relapse, or not worsening RRMS. A portion of RRMS diagnosed subject will progress to the SPMS disease course, which is characterized by a progressive worsening of neurologic function, i.e. an accumulation of disability, over time. SPMS subclassifications can be made such as active (relapses and/or new MRI activity), not active, progressive (disease worsening over time), or non-progressive SPMS. Finally, PPMS is an MS disease course characterized by worsening of neurologic function and hence an accumulation of disability from the onset of symptoms, without early relapse or remission. Further PPMS subgroups can be formed such as active PPMS (occasional relapse and/or new MRI activity), non-active PPMS, progressive PPMS (evidence of disease worsening over time, regardless of new MRI activity) and non-progressive PPMS. In general, MS disease courses are characterized by substantial intersubject variability in terms of relapse and remission periods, both in severity (in case of relapse) and duration.

Several disease modifying therapies are available for MS, and therefore the current invention may be used as alternative treatment strategy, or in combination with these existing therapies. Non-limiting examples of active pharmaceutical ingredients include interferon beta-1a, interferon beta-1b, glatiramer acetate, glatiramer acetate, peginterferon beta-1a, teriflunomide, fingolimod, cladribine, siponimod, dimethyl fumarate, diroximel fumarate, ozanimod, alemtuzumab, mitoxantrone, ocrelizumab, and natalizumab. Alternatively, the invention may be used in combination with a treatment or medication aiming to relapse management, such as but not limited to methylprednisolone, prednisone, and adrenocorticotropic hormone(s) (ACTH). Further, the invention may be used in combination with a therapy aiming to alleviate specific symptoms. Non-limiting examples include medications aiming to improve or avoid symptoms selected from the group consisting of: bladder problems, bowel dysfunction, depression, dizziness, vertigo, emotional changes, fatigue, itching, pain, sexual problems, spasticity, tremors, and walking difficulties.

MS is characterized by three intertwined hallmark characteristics: 1) lesion formation in the central nervous system, 2) inflammation, and 3) degradation of myelin sheaths of neurons. Despite traditionally being considered a demyelinating disease of the central nervous system and white matter, more recently reports have surfaced that demyelination of the cortical and deep gray matter may exceed white matter demyelination (Kutzelnigg et al. (2005). Brain. 128(11), 2705-2712). Two main hypotheses have been postulated as to how MS is caused at the molecular level. The commonly accepted “outside-in hypothesis” is based on the activation of peripheral autoreactive effector CD4+ T cells which migrate to the central nervous system and initiate the disease process. Once in the central nervous system, said T cells are locally reactivated by APCs and recruit additional T cells and macrophages to establish inflammatory lesions. Noteworthy, MS lesions have been shown to contain CD8+ T cells predominantly found at the lesion edges, and CD4+ T cells found more central in the lesions. These cells are thought to cause demyelination, oligodendrocyte destruction, and axonal damage, leading to neurologic dysfunction. Additionally, immune-modulatory networks are triggered to limit inflammation and to initiate repair, which results in at least partial remyelination reflected by clinical remission. Nonetheless, without adequate treatment, further attacks often lead to progression of the disease.

MS onset is believed to originate well before the first clinical symptoms are detected, as evidenced by the typical occurrence of apparent older and inactive lesions on the MRI of patients. Due to advances in the development of diagnostic methods, MS can now be detected even before a clinical manifestation of the disease (i.e. pre-symptomatic MS). In the context of the invention, “treatment of MS” and similar expressions envisage treatment of, and treatment strategies for, both symptomatic and pre-symptomatic MS.

In particular, when the immunogenic peptides and/or resulting cytolytic CD4+ T cells are used for treating a pre-symptomatic MS patient, the disease is halted at such an early stage that clinical manifestations may be partially, or even completely avoided. MS wherein the subject is not fully responsive to a treatment of interferon beta is also encompassed within the term “MS”. The main antigens attacked by the immune system and leading to the disease are: Myelin oligodendrocyte glycoprotein (MOG), Myelin basic protein (MBP), Proteolipid protein (PLP), Myelin-oligodendrocytic basic protein (MOBP), and Oligodendrocyte-specific protein (OSP).

The term “Neuromyelitis Optica” or “NMO” and “NMO Spectrum Disorder (NMOSD)”, also known as “Devic's disease”, refers to an autoimmune disorder in which white blood cells and antibodies primarily attack the optic nerves and the spinal cord, but may also attack the brain (reviewed in Wingerchuk 2006, Int MS J. 2006 May; 13(2):42-50). The damage to the optic nerves produces swelling and inflammation that cause pain and loss of vision; the damage to the spinal cord causes weakness or paralysis in the legs or arms, loss of sensation, and problems with bladder and bowel function. NMO is a relapsing-remitting disease. During a relapse, new damage to the optic nerves and/or spinal cord can lead to accumulating disability. Unlike MS, there is no progressive phase of this disease. Therefore, preventing attacks is critical to a good long-term outcome. In cases associated with anti-MOG antibodies, it is considered that anti-MOG antibodies may trigger an attack on the myelin sheath resulting in demyelination. The cause of NMO in the majority of cases is due to a specific attack on auto-antigens. Up to a third of subjects may be positive for auto-antibodies directed against a component of myelin called myelin oligodendrocyte glycoprotein (MOG). People with anti-MOG related NMO similarly have episodes of transverse myelitis and optic neuritis.

The term “Rheumatoid Arthritis” or “RA” is an autoimmune, inflammatory disease that causes pain, swelling, stiffness, and loss of function in various joints (most commonly in the hands, wrists, and knees). The respective joint's lining becomes inflamed, leading to tissue damage, as well as chronic pain, unsteadiness, and deformity. There is generally a bilateral/symmetrical pattern of disease progression (e.g., both hands or both knees are affected). RA can also affect extra-articular sites, including the eyes, mouth, lungs, and heart. Patients can experience an acute worsening of their symptoms (called a flare) but with early intervention and appropriate treatment, symptoms can be ameliorated for a certain duration (reviewed by Sana Iqbal et al., 2019, US Pharm. 2019; 44(1)(Specialty&Oncology suppl):8-11). The antigens attacked by the immune system and responsible for the disease are diverse but some examples are: GRP78, HSP60, 60 kDa chaperonin 2, Gelsolin, Chitinase-3-like protein 1, Cathepsin S, Serum albumin, and Cathepsin D.

The term “type 1 diabetes” (T1D) or “diabetes type 1” (also known as “type 1 diabetes mellitus” or “immune mediated diabetes” or formerly known as “juvenile onset diabetes” or “insulin dependent diabetes”) is an autoimmune disorder that typically develops in susceptible individuals during childhood. At the basis of T1D pathogenesis is the destruction of most insulin-producing pancreatic beta-cells by an autoimmune mechanism. In short, the organism loses the immune tolerance towards the pancreatic beta-cells in charge of insulin production and induces an immune response, mainly cell-mediated, associated to the production of autoantibodies, which leads to the self-destruction of beta-cells. The main antigen attacked by the immune system and responsible for the disease is (pro)insulin but other examples are: GAD65, GAD67, IA-2 (ICA512), IA-2 (beta/phogrin), IGRP, Chromogranin, ZnT8 and HSP-60.

The term “therapeutically effective amount” refers to an amount of the non-immunogenic RNA molecule encoding the peptide of the invention or derivative thereof, which produces the desired therapeutic or preventive effect in a patient. For example, in reference to a disease or disorder, it is the amount which reduces to some extent one or more symptoms of the disease or disorder, and more particularly returns to normal, either partially or completely, the physiological or biochemical parameters associated with or causative of the disease or disorder. Typically, the therapeutically effective amount is the amount of the non-immunogenic RNA of the invention or derivative thereof, which will lead to an improvement or restoration of the normal physiological situation.

The RNA is preferably administered through intramuscular injection of the RNA in a suitable buffer comprising sodium chloride.

The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence.

In this context, it is realised that peptide fragments are generated from antigens, typically in the context of epitope scanning. By coincidence such peptides may comprise in their sequence a T cell epitope (an MHC class II epitope or a CD1d binding epitope) and in their proximity a sequence with the modified oxidoreductase motif as defined herein. Alternatively, there can be an amino acid sequence of at most 11 amino acids, at most 7 amino acids, at most 4 amino acids, at most 2 amino acids between said epitope and said oxidoreductase motif, or even 0 amino acids (in other words the epitope and oxidoreductase motif sequence are immediately adjacent to each other). In preferred embodiment, such naturally occurring peptides are disclaimed.

Amino acids are referred to herein with their full name, their three-letter abbreviation or their one letter abbreviation.

Motifs of amino acid sequences are written herein according to the format of Prosite. Motifs are used to describe a certain sequence variety at specific parts of a sequence. The symbol “X” is used for a position where any amino acid is accepted. Alternatives are indicated by listing the acceptable amino acids for a given position, between square brackets (‘[ ]’). For example: [CST] stands for an amino acid selected from Cys, Ser or Thr. Amino acids which are excluded as alternatives are indicated by listing them between curly brackets (‘{ }’). For example: {AM}stands for any amino acid except Ala and Met. The different elements in a motif are optionally separated from each other by a hyphen (-). In the context of the motifs disclosed in this specification, the disclosed general oxidoreductase motifs are typically accompanied by a hyphen not forming a connection with a different element outside the motif. These ‘open’ hyphens indicate the position of the physical connection of the motif with another portion of the immunogenic peptide such as a linker sequence or an epitope sequence. For example, a motif of the form “Zₘ—C—Xₙ—[CST]” indicates that the [CST] is the amino acid connected to the other portion of the immunogenic peptide, and Z is a terminal amino acid of the immunogenic peptide. Preferred physical connections are peptide bonds. Repetition of an identical element within a motif can be indicated by placing behind that element a numerical value or a numerical range between parentheses. In this respect, “Xₙ” refers to n-times “X”. For example X(2) corresponds to X-X or XX; X(2, 5) corresponds to 2, 3, 4 or 5 X amino acids, A(3) corresponds to A-A-A or AAA. To distinguish between the amino acids, those outside the oxidoreductase motif can be called external amino acids, those within the oxidoreductase motif are called internal amino acids. Unless stated otherwise X represents any amino acid, particularly an L-amino acid, more particularly one of the 20 naturally occurring L-amino acids.

Non-immunogenic RNA encoding a peptide, comprising a T cell epitope, e.g. an MHC class II T-cell epitope or an NKT-cell epitope (or CD1d binding peptide epitope) and a modified peptide motif sequence, having reducing activity is capable of generating a population of antigen-specific cytolytic CD4+ T-cells, respectively cytolytic NKT-cells towards antigen-presenting cells.

Accordingly, in its broadest sense, the invention relates to non-immunogenic RNA encoding peptides which comprise at least one T-cell epitope (MHC class II T-cell epitope or an NKT-cell epitope) of an antigen (self or non-self) with a potential to trigger an immune reaction, and a modified oxidoreductase sequence motif with a reducing activity on peptide disulfide bonds. The T cell epitope and the modified oxidoreductase motif sequence may be immediately adjacent to each other in the peptide or optionally separated by one or more amino acids (so called linker sequence). Optionally the peptide additionally comprises an endosome targeting sequence and/or additional “flanking” sequences.

The non-immunogenic RNA encodes peptides that comprise a T-cell epitope of an antigen (self or non self) with a potential to trigger an immune reaction, and a modified oxidoreductase motif. The reducing activity of the motif sequence in the peptide can be assayed for its ability to reduce a sulfhydryl group such as in the insulin solubility assay wherein the solubility of insulin is altered upon reduction, or with a fluorescence-labelled substrate such as insulin. An example of such assay uses a fluorescent peptide and is described in Tomazzolli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

The modified oxidoreductase motif may be positioned at the amino-terminus side of the T-cell epitope or at the carboxy-terminus of the T-cell epitope.

Peptide fragments with reducing activity are encountered in thioreductases which are small disulfide reducing enzymes including glutaredoxins, nucleoredoxins, thioredoxins and other thiol/disulfide oxidoreductases (Holmgren (2000) Antioxid. Redox Signal 2, 811-820; Jacquot et al (2002) Biochem. Pharm. 64, 1065-1069). They are multifunctional, ubiquitous and found in many prokaryotes and eukaryotes. They are known to exert reducing activity for disulfide bonds on proteins (such as enzymes) through redox active cysteines within conserved active domain consensus sequences well-known from e.g. Fomenko et al ((2003) Biochemistry 42, 11214-11225; Fomenko et al (2002) Prot. Science 11, 2285-2296), in which X stands for any amino acid. and WO2008/017517 comprising a cysteine at position 1 and/or 4. Thus the motif there is either CXX[CST](SEQ ID NO: 111) or [CST]XXC (SEQ ID NO: 110). Such domains are also found in larger proteins such as protein disulfide isomerase (PDI) and phosphoinositide-specific phospholipase C. The present invention has redesigned said motifs in search for more potency and activity.

The terms “cysteine”, “C”, “serine”, “S”, and “threonine”, “T”, when used in the light of the amino acid residues present in the oxidoreductase motifs disclosed herein respectively refer to naturally occurring cysteine, serine or threonine amino acids. Unless explicitly stated differently, said terms hence exclude chemically modified cysteines, serines and threonines such as those modified so as to carry an acetyl, methyl, ethyl or propionyl group, either on the N-terminal amide of the amino acid residue of the motif or on the C-terminal carboxy group.

In the peptides encoded by the non-immunogenic RNA of the present invention, the oxidoreductase motif is located such that, when the epitope fits into the MHC groove, the oxidoreductase motif remains outside of the MHC binding groove. The oxidoreductase motif is placed either immediately adjacent to the epitope sequence within the peptide [in other words a linker sequence of zero amino acids between motif and epitope], or is separated from the T cell epitope by a linker comprising an amino acid sequence of 5 amino acids or less. More particularly, the linker comprises 1, 2, 3, 4, or 5 amino acids. Specific embodiments are peptides with a 0, 1, 2 or 3 amino acid linker between epitope sequence and modified oxidoreductase motif sequence. Apart from a peptide linker, other organic compounds can be used as linker to link the parts of the peptide to each other (e.g. the modified oxidoreductase motif sequence to the T cell epitope sequence).

The peptides encoded by the non-immunogenic RNA of the present invention can further comprise additional short amino acid sequences N- or C-terminally of the sequence comprising the T cell epitope and the oxidoreductase motif. Such an amino acid sequence is generally referred to herein as a ‘flanking sequence’. A flanking sequence can be positioned between the epitope and an endosomal targeting sequence and/or between the modified oxidoreductase motif and an endosomal targeting sequence. In certain peptides, not comprising an endosomal targeting sequence, a short amino acid sequence may be present N- and/or C-terminally of the modified oxidoreductase motif and/or epitope sequence in the peptide. More particularly a flanking sequence is a sequence of between 1 and 7 amino acids, most particularly a sequence of 2 amino acids.

In certain embodiments of the present invention, the non-immunogenic RNA encodes peptides comprising one T-cell epitope sequence and a single oxidoreductase motif sequence. Alternatively, oxidoreductase motifs can be provided at both the N- and the C-terminus of the T cell epitope sequence.

Other variations envisaged for the peptides encoded by the non-immunogenic RNA of the present invention include peptides which contain repeats of a T cell epitope sequence wherein each epitope sequence is preceded and/or followed by the modified oxidoreductase motif (e.g. repeats of “oxidoreductase motif-epitope” or repeats of “oxidoreductase motif-epitope-oxidoreductase motif”). Herein the oxidoreductase motifs can all have the same sequence but this is not obligatory.

Typically, the peptides encoded by the non-immunogenic RNA of the present invention comprise only one T cell epitope. As described below a T cell epitope in a protein sequence can be identified by functional assays and/or one or more in silica prediction assays. The amino acids in a T cell epitope sequence are numbered according to their position in the binding groove of the MHC proteins or bind the CD1d molecule. An MHC class II T-cell epitope present within a peptide typically consists of between 7 and 30 amino acids, preferably between 9 and 30 amino acids, such as of between 9 and 25 amino acids, yet more particularly of between 9 and 16 amino acids, yet most particularly consists of 9, 10, 11, 12, 13, 14, 15 or 16 amino acids. An NKT cell epitope present within a peptide typically consists of between 7 and 30 amino acids, such as of between 7 and 25 amino acids, yet more particularly of between 7 and 16 amino acids, yet most particularly consists of 7, 8, 9, 10, 11, 12, 13, 14, 15 or 16 amino acids.

In a more particular embodiment, the T cell epitope consists of a sequence of 9, 10, or 11 amino acids. In a further particular embodiment, the T-cell epitope is an epitope, which is presented to T cells by MHC-class II molecules [MHC class II restricted T cell epitopes]. Typically, T cell epitope sequence refers to the octapeptide or more specifically nonapeptide sequence which fits into the cleft of an MHC II protein.

In a more particular embodiment, the T cell epitope consists of a sequence of 7, 8, or 9 amino acids. In a further particular embodiment, the T-cell epitope is an epitope, which is presented by CD1d molecules [NKT cell epitopes]. Typically, NKT cell epitope sequence refers to the 7 amino acid peptide sequence which binds to and is presented by the CD1d protein.

The T cell epitope of the peptides encoded by the non-immunogenic RNA of the present invention can correspond either to a natural epitope sequence of a protein or can be a modified version thereof, provided the modified T cell epitope retains its ability to bind within the MHC cleft or to bind the CD1d receptor, similar to the natural T cell epitope sequence. The modified T cell epitope can have the same binding affinity for the MHC protein or the CD1d receptor as the natural epitope, but can also have a lowered affinity. In particular, the binding affinity of the modified peptide is no less than 10-fold less than the original peptide, more particularly no less than 5 times less. Peptides encoded by the non-immunogenic RNA of the present invention have a stabilising effect on protein complexes. Accordingly, the stabilising effect of the peptide-MHC or CD1d complex compensates for the lowered affinity of the modified epitope for the MHC or CD1d molecule.

In the peptides encoded by the non-immunogenic RNA of the present invention, the sequence comprising the T cell epitope and the reducing compound within the peptide can be further linked to an amino acid sequence (or another organic compound) that facilitates uptake of the peptide into late endosomes for processing and presentation within MHC class II determinants. The late endosome targeting is mediated by signals present in the cytoplasmic tail of proteins and corresponds to well-identified peptide motifs. The late endosome targeting sequences allow for processing and efficient presentation of the antigen-derived T cell epitope by MHC-class II molecules. Such endosomal targeting sequences are contained, for example, within the gp75 protein (Vijayasaradhi et al. (1995) J. Cell. Biol. 130, 807-820), the human CD3 gamma protein, the HLA-BM 11 (Copier et al. (1996) J. Immunol. 157, 1017-1027), the cytoplasmic tail of the DEC205 receptor (Mahnke et al. (2000) J. Cell Biol. 151, 673-683). Other examples of peptides which function as sorting signals to the endosome are disclosed in the review of Bonifacio and Traub (2003) Annu. Rev. Biochem. 72, 395-447. Alternatively, the sequence can be that of a subdominant or minor T cell epitope from a protein, which facilitates uptake in late endosome without overcoming the T cell response towards the antigen. The late endosome targeting sequence can be located either at the amino-terminal or at the carboxy-terminal end of the antigen derived peptide for efficient uptake and processing and can also be coupled through a flanking sequence, such as a peptide sequence of up to 10 amino acids. When using a minor T cell epitope for targeting purpose, the latter is typically located at the amino-terminal end of the antigen derived peptide.

Alternatively, the present invention relates to the production of non-immunogenic RNA encoding peptides comprising an NKT epitope as defined herein containing hydrophobic residues that confer the capacity to bind to the CD1d molecule. Upon administration, such RNA is targeted to (immature) dendritic cells, wherein it is translated into peptides directed to the late endosome where they are loaded onto CD1d and presented at the surface of the APC.

Accordingly, the present invention envisages non-immunogenic RNA encoding peptides of antigenic proteins and its use in eliciting specific immune reactions. These peptides can either correspond to fragments of proteins which comprise, within their sequence i.e. a reducing compound and a T cell epitope separated by at most 10, preferably 7 amino acids or less. Alternatively, and for most antigenic proteins, the peptides encoded by the non-immunogenic RNA of the invention comprise a reducing compound, more particularly a reducing modified oxidoreductase motif as described herein, coupled N-terminally or C-terminally to a T cell epitope of the antigenic protein (either directly adjacent thereto or with a linker of at most 10, more particularly at most 7 amino acids). Moreover, the T cell epitope sequence of the protein and/or the modified oxidoreductase motif can be modified and/or one or more flanking sequences and/or a targeting sequence can be introduced (or modified), compared to the naturally occurring sequence. Thus, depending on whether or not the features of the present invention can be found within the sequence of the antigenic protein of interest, the non-immunogenic RNA encodes peptides that can comprise a sequence which is ‘artificial’ or ‘naturally occurring’.

The peptides encoded by the non-immunogenic RNA of the invention can vary substantially in length. The length of the peptides can vary from 9, 10 or 11 amino acids, i.e. consisting of an NKT cell or MHC class II T cell epitope of respectively 7, 8 or 9 amino acids, adjacent thereto the minimal oxidoreductase motif of 2 amino acids (CC), to up to 12, 13, 14, 15, 16, 17, 18, 19, 20, 25, 30, 35, 40, 45 or up to 50 amino acids. In particular embodiments, the complete peptide encoded by the non-immunogenic RNA of the invention consists of between 9 amino acids up 20, 25, 30, 40, 50, 75 or 100 amino acids. For example, a peptide encoded by the non-immunogenic RNA of the invention may comprise an endosomal targeting sequence of 40 amino acids, a flanking sequence of about 2 amino acids, an oxidoreductase motif as described herein of between 2 and about 11 amino acids, a linker of 4 to 7 amino acids and a T cell epitope peptide of minimally 7, 8 or 9 amino acids. More particularly, where the reducing compound is a modified oxidoreductase motif as described herein, the length of the (artificial or natural) sequence comprising the epitope and modified oxidoreductase motif optionally connected by a linker (referred to herein as ‘epitope-modified oxidoreductase motif’ sequence), without the endosomal targeting sequence, is critical. The ‘epitope-modified oxidoreductase motif’ more particularly has a length of 9, 10, 11, 12, 13, 14, 15, 16, 17, 18 or 19 amino acids. Such peptides of 9, 10, 11, 12, 13 or 14 to 19 amino acids can optionally be coupled to an endosomal targeting signal of which the size is less critical. The late endosome targeting is mediated by signals present in the cytoplasmic tail of proteins and correspond to well-identified peptide motifs such as the dileucine-based [DE]XXXL[LI](SEQ ID NO: 110) or DXXLL (SEQ ID NO: 111) motif (e.g. DXXXLL (SEQ ID NO: 112)), the tyrosine-based YXXO (SEQ ID NO: 113) motif or the so-called acidic cluster motif. The symbol 0 represents amino acid residues with a bulky hydrophobic side chain such as Phe, Tyr and Trp. The late endosome targeting sequences allow for processing and efficient presentation of the antigen-derived T cell epitope by MHC class II or CD1d molecules.

As detailed above, in particular embodiments, the peptides encoded by the non-immunogenic RNA of the present invention comprise a reducing modified oxidoreductase motif as described herein linked to a T cell epitope sequence.

In further particular embodiments, the peptides encoded by the non-immunogenic RNA of the invention are peptides comprising T cell epitopes which do not comprise an amino acid sequence with redox properties within their natural sequence.

However, in alternative embodiments, the T cell epitope may comprise any sequence of amino acids ensuring the binding of the epitope to the MHC cleft or to the CD1d molecule. Where an epitope of interest of an antigenic protein comprises a modified oxidoreductase motif such as described herein within its epitope sequence, the peptides encoded by the non-immunogenic RNA of the invention comprise the sequence of an oxidoreductase motif as described herein and/or of another reducing sequence coupled N- or C-terminally to the epitope sequence such that (contrary to the modified oxidoreductase motif present within the epitope, which is buried within the cleft) the attached oxidoreductase motif can ensure the reducing activity.

Accordingly, the T cell epitope and motif are immediately adjacent or separated from each other and do not overlap. To assess the concept of “immediately adjacent” or “separated”, the 7, 8 or 9 amino acid sequence which fits in the MHC cleft or CD1d molecule is determined and the distance between this octapeptide or nonapeptide with the modified oxidoreductase motif is determined.

Generally, the peptides encoded by the non-immunogenic RNA of the invention are not natural (thus no fragments of proteins as such) but artificial peptides which contain, in addition to a T cell epitope, a modified oxidoreductase motif as described herein, whereby the modified oxidoreductase motif is immediately separated from the T cell epitope by a linker consisting of up to seven, most particularly up to four or up to 2 amino acids.

It has been previously shown that upon administration (i.e. injection) to a mammal of a peptide comprising an oxidoreductase motif and an MHC class II T-cell epitope (or a composition comprising such RNA), the peptide elicits the activation of T cells recognising the antigen derived T cell epitope and provides an additional signal to the T cell through reduction of surface receptor. This supra-optimal activation results in T cells acquiring cytolytic properties for the cell presenting the T cell epitope, as well as suppressive properties on bystander T cells. The present invention builds further on this principle by administering non-immunogenic RNA encoding such peptides rather than the peptide as such.

Additionally, it has been shown that upon administration (i.e. injection) to a mammal of a peptide comprising an oxidoreductase motif and an NKT-cell epitope (or a composition comprising such a peptide), the peptide elicits the activation of T cells recognising the antigen derived T cell epitope and provides an additional signal to the T cell through binding to the CD1d surface receptor. This activation results in NKT cells acquiring cytolytic properties for the cell presenting the T cell epitope. The present invention builds further on this principle by administering non-immunogenic RNA encoding such peptides rather than the peptide as such.

In this way, the non-immunogenic RNA encoding such peptides or compositions comprising the RNA as described in the present invention, can be used for direct immunisation of mammals, including human beings. The invention thus provides non-immunogenic RNA encoding such peptides of the invention, or compositions comprising such RNA for use as a medicine. Accordingly, the present invention provides therapeutic methods which comprise administering non-immunogenic RNA encoding one or more peptides according to the present invention or compositions comprising such RNA to a patient in need thereof.

The present invention offers methods by which antigen-specific T cells endowed with cytolytic properties can be elicited by immunisation with RNA encoding small peptides as described herein. It has been found that peptides which contain (i) a sequence encoding a T cell epitope from an antigen and (ii) a consensus sequence with redox properties, and further optionally also comprising a sequence to facilitate the uptake of the peptide into late endosomes for efficient MHC-class II presentation or CD1d receptor binding, elicit cytolytic CD4+ T-cells or NKT cells respectively. The present invention builds further on this principle by administering non-immunogenic RNA encoding such peptides rather than the peptide as such.

The properties of the peptides encoded by the non-immunogenic RNA of the present invention are of particular interest in the treatment and prevention of (auto)immune reactions.

Non-immunogenic RNA encoding peptides described herein or compositions comprising such RNA are used as medicament, more particularly used for the manufacture of a medicament for the prevention or treatment of an immune disorder in a mammal, more in particular in a human.

The present invention describes methods of treatment or prevention of an immune disorder of a mammal in need for such treatment or prevention, comprising administering non-immunogenic RNA encoding the peptides as described herein. The methods comprising the step of administering to said mammal suffering or at risk of an immune disorder a therapeutically effective amount of the non-immunogenic RNA encoding peptides of the invention, homologues or derivatives thereof such as to reduce the symptoms of the immune disorder. The treatment of both humans and animals, such as, pets and farm animals is envisaged. In an embodiment the mammal to be treated is a human. The immune disorders referred to above are in a particular embodiment selected from allergic diseases and autoimmune diseases.

The non-immunogenic RNA encoding the peptides as described herein or the pharmaceutical composition comprising such RNA as defined herein is preferably administered through sub-cutaneous or intramuscular administration.

In one embodiment, the RNA or pharmaceutical compositions comprising such can be injected sub-cutaneously (SC) in the region of the lateral part of the upper arm, midway between the elbow and the shoulder. When two or more separate injections are needed, they can be administered concomitantly in both arms.

In one embodiment, the RNA or pharmaceutical compositions comprising such can be injected intra-muscularly (IM) in the region of the lateral part of the upper arm, preferably in the deltoid muscle of arm. When two or more separate injections are needed, they can be administered concomitantly in both arms.

The non-immunogenic RNA encoding a peptide according to the invention or the pharmaceutical composition comprising such RNA is administered in a therapeutically effective dose.

The oxidoreductase motif in the peptides encoded by the non-immunogenic RNA of the present invention is defined by the following general formula:
Zₘ—[CST]-Xₙ—C— or Zₘ—C—Xₙ—[CST]-, as defined in the aspects described herein, wherein the hyphen (-) in said oxidoreductase motif indicates the point of attachment of the oxidoreductase motif to the N-terminal end of the linker or the epitope, or to the C-terminal end of the linker or the T cell epitope.

In a preferred embodiment, said oxidoreductase motif is CC or CXC, wherein X can be any amino acid selected from the group consisting of: G, A, V, L, I, M, F, W, P, S, T, C, Y, N, Q, D, E, K, R, and H, or non-natural basic amino acids. Preferably, X in the CXC motif is any amino acid except for C, S, or T. In a specific embodiment, X in the CXC motif is a basic amino acid, such as H, K, or R, or a non-natural basic amino acid such as, but not limited to:
lysine variants like Fmoc-β-Lys(Boc)-OH (CAS Number 219967-68-7), Fmoc-Orn(Boc)-OH also called L-ornithine or ornithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)—OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

Specific examples of the CXC motif are: CHC, CKC, CRC, CGC, CAC, CVC, CLC, CIC, CMC, CFC, CWC, CPC, CSC, CTC, CYC, CNC, CQC, CDC, and CEC. Any one of these exemplary CXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein. Preferred examples of such motifs are: KCHC, KCKC, KCRC, KCGC, KCAC, KCVC, KCLC, KCIC, KCMC, KCFC, KCWC, KCPC, KCSC, KCTC, KCYC, KCNC, KCQC, KCDC, KCEC, HCHC, HCKC, HCRC, HCGC, HCAC, HCVC, HCLC, HCIC, HCMC, HCFC, HCWC, HCPC, HCSC, HCTC, HCYC, HCNC, HCQC, HCDC, HCEC, RCHC, RCKC, RCRC, RCGC, RCAC, RCVC, RCLC, RCIC, RCMC, RCFC, RCWC, RCPC, RCSC, RCTC, RCYC, RCNC, RCQC, RCDC, and RCEC (SEQ ID NO: 114 to 170);

In a preferred embodiment, said oxidoreductase motif is CX₂C (SEQ ID NO: 171), i.e. CXXC, typically CX¹X²C, wherein X¹, and X², each individually can be any amino acid selected from the group consisting of: G, A, V, L, I, M, F, W, P, S, T, C, Y, N, Q, D, E, K, R, and H, or non-natural basic amino acids as defined herein. Preferably, X¹, and X² in said motif is any amino acid except for C, S, or T. In a specific embodiment, at least one of X¹ or X² in said motif is a basic amino acid, such as H, K, or R, or a non-natural basic amino acid as defined herein.

Specific examples of the X¹X² amino acid couple in said ZₘCX¹X²C motif are: PY, HY, KY, RY, PH, PK, PR, HG, KG, RG, HH, HK, HR, GP, HP, KP, RP, GH, GK, GR, GH, KH, and RH.

Any one of these exemplary C X¹X²C motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

Specific examples of the ZₘCX¹X²C motif are: HCX¹X²C, KHCX¹X²C, KCX¹X²C, RCX¹X²C, HCX¹X²C, KCX¹X²C, RCX¹X²C, KKCX¹X²C, KRCX¹X²C, KHCX¹X²C, KKCX¹X²C, and KRCX¹X²C (corresponding to SEQ ID NOs: 172 to 183);

More specific examples of the ZₘCXXC motif are: CPYC (SEQ ID NO: 98), HCPYC, KHCPYC, KCPYC, RCPYC, HCGHC, KCGHC, RCGHC, KKCPYC, KRCPYC, KHCGHC, KKCGHC, and KRCGHC (SEQ ID NOs: 24 to 35);

In a preferred embodiment, said oxidoreductase motif is CX₃C (SEQ ID NO: 184), i.e. CXXXC, typically CX¹X²X³C, wherein X¹, X², and X³, each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², and X³ in said motif is any amino acid except for C, S, or T. In a specific embodiment, at least one of X¹, X², or X³ in said motif is a basic amino acid, such as H, K, or R, or a non-natural basic amino acid as defined herein.

Specific examples of the CXXXC motif are: CXPYC, CPXYC, and CPYXC, wherein X can be any amino acid, more preferably:

CXPYC, such as: CKPYC, CRPYC, CHPYC, CGPYC, CAPYC, CVPYC, CLPYC, CIPYC, CMPYC, CFPYC, CWPYC, CPPYC, CSPYC, CTPYC, CCPYC, CYPYC, CNPYC, CQPYC, CDPYC, and CEPYC (SEQ ID NO: 185 to 205); or

CPXYC, such as: CPKYC, CPRYC, CPHYC, CPGYC, CPAYC, CPVYC, CPLYC, CPIYC, CPMYC, CPFYC, CPWYC, CPPYC, CPSYC, CPTYC, CPCYC, CPYYC, CPNYC, CPQYC, CPDYC, CPEYC, and CPLYC (SEQ ID NO: 206 to 227); or

CPYXC, such as: CPYKC, CPYRC, CPYHC, CPYGC, CPYAC, CPYVC, CPYLC, CPYIC, CPYMC, CPYFC, CPYWC, CPYPC, CPYSC, CPYTC, CPYCC, CPYYC, CPYNC, CPYQC, CPYDC, CPYEC, and CPYLC (SEQ ID NO: 228 to 249).

Further specific examples of the CXXXC motif are: CXHGC, CHXGC, and CHGXC, wherein X can be any amino acid, more preferably:

CXHGC, such as: CKHGC, CRHGC, CHHGC, CGHGC, CAHGC, CVHGC, CLHGC, CIHGC, CMHGC, CFHGC, CWHGC, CPHGC, CSHGC, CTHGC, CCHGC, CYHGC, CNHGC, CQHGC, CDHGC, CEHGC, and CKHGC (SEQ ID NO: 250 to 271); or

CGXHC, such as: CGKHC, CGRHC, CGHHC, CGGHC, CGAHC, CGVHC, CGLHC, CGIHC, CGMHC, CGFHC, CGWHC, CGPHC, CGSHC, CGTHC, CGCHC, CGYHC, CGNHC, CGQHC, CGDHC, CGEHC, and CGLHC (SEQ ID NO: 272 to 293); or

CHGXC, such as: CHGKC, CHGRC, CHGHC, CHGGC, CHGAC, CHGVC, CHGLC, CHGIC, CHGMC, CHGFC, CHGWC, CHGPC, CHGSC, CHGTC, CHGCC, CHGYC, CHGNC, CHGQC, CHGDC, CHGEC, and CHGLC (SEQ ID NO: 294 to 315).

Further specific examples of the CXXXC motif are: CXGPC, CGXPC, and CGPXC, wherein X can be any amino acid, more preferably:

CXGPC, such as: CKGPC, CRGPC, CHGPC, CGGPC, CAGPC, CVGPC, CLGPC, CIGPC, CMGPC, CFGPC, CWGPC, CPGPC, CSGPC, CTGPC, CCGPC, CYGPC, CNGPC, CQGPC, CDGPC, CEGPC, and CKGPC (SEQ ID NO: 316 to 337); or

CGXPC, such as: CGKPC, CGRPC, CGHPC, CGGPC, CGAPC, CGVPC, CGLPC, CGIPC, CGMPC, CGFPC, CGWPC, CGPPC, CGSPC, CGTPC, CGCPC, CGYPC, CGNPC, CGQPC, CGDPC, CGEPC, and CGLPC (SEQ ID NO: 338 to 359); or

CGPXC, such as: CGPKC, CGPRC, CGPHC, CGPGC, CGPAC, CGPVC, CGPLC, CGPIC, CGPMC, CGPFC, CGPWC, CGPPC, CGPSC, CGPTC, CGPCC, CGPYC, CGPNC, CGPQC, CGPDC, CGPEC, and CGPLC (SEQ ID NO: 360 to 381).

Further specific examples of the CXXXC motif are: CXGHC, CGXHC, and CGHXC, wherein X can be any amino acid, more preferably:

CXGHC, such as: CKGHC, CRGHC, CHGHC, CGGHC, CAGHC, CVGHC, CLGHC, CIGHC, CMGHC, CFGHC, CWGHC, CPGHC, CSGHC, CTGHC, CCGHC, CYGHC, CNGHC, CQGHC, CDGHC, CEGHC, and CKGHC (SEQ ID NO: 382 to 403); or

CGXFC, such as: CGKFC, CGRFC, CGHFC, CGGFC, CGAFC, CGVFC, CGLFC, CGIFC, CGMFC, CGFFC, CGWFC, CGPFC, CGSFC, CGTFC, CGCFC, CGYFC, CGNFC, CGQFC, CGDFC, CGEFC, and CGLFC (SEQ ID NO: 404 to 425); or

CGHXC, such as: CGHKC, CGHRC, CGHHC, CGHGC, CGHAC, CGHVC, CGHLC, CGHIC, CGHMC, CGHFC, CGHWC, CGHPC, CGHSC, CGHTC, CGHCC, CGHYC, CGHNC, CGHQC, CGHDC, CGHEC, and CGHLC (SEQ ID NO: 426 to 447).

Further specific examples of the CXXXC motif are: CXGFC, CGXFC, and CGFXC, wherein X can be any amino acid, more preferably:

CXGFC, such as: CKGFC, CRGFC, CHGFC, CGGFC, CAGFC, CVGFC, CLGFC, CIGFC, CMGFC, CFGFC, CWGFC, CPGFC, CSGFC, CTGFC, CCGFC, CYGFC, CNGFC, CQGFC, CDGFC, CEGFC, and CKGFC (SEQ ID NO: 448 to 469); or

CGXFC, such as: CGKFC, CGRFC, CGHFC, CGGFC, CGAFC, CGVFC, CGLFC, CGIFC, CGMFC, CGFFC, CGWFC, CGPFC, CGSFC, CGTFC, CGCFC, CGYFC, CGNFC, CGQFC, CGDFC, CGEFC, and CGLFC (SEQ ID NO: 470 to 491); or

CGFXC, such as: CGFKC, CGFRC, CGFHC, CGFGC, CGFAC, CGFVC, CGFLC, CGFIC, CGFMC, CGFFC, CGFWC, CGFPC, CGFSC, CGFTC, CGFCC, CGFYC, CGFNC, CGFQC, CGFDC, CGFEC, and CGFLC (SEQ ID NO: 492 to 513).

Further specific examples of the CXXXC motif are: CXRLC, CRXLC, and CRLXC, wherein X can be any amino acid, more preferably:

CXRLC, such as: CKRLC, CRRLC, CHRLC, CGRLC, CARLC, CVRLC, CLRLC, CIRLC, CMRLC, CFRLC, CWRLC, CPRLC, CSRLC, CTRLC, CCRLC, CYRLC, CNRLC, CQRLC, CDRLC, CERLC, and CKRLC (SEQ ID NO: 514 to 535); or

CRXLC, such as: CRKLC, CRRLC, CRHLC, CRGLC, CRALC, CRVLC, CRLLC, CRILC, CRMLC, CRFLC, CRWLC, CRPLC, CRSLC, CRTLC, CRCLC, CRYLC, CRNLC, CRQLC, CRDLC, CRELC, and CRLLC (SEQ ID NO: 536 to 557); or

CRLXC, such as: CRLKC, CRLRC, CRLHC, CRLGC, CRLAC, CRLVC, CRLLC, CRLIC, CRLMC, CRLFC, CRLWC, CRLPC, CRLSC, CRLTC, CRLCC, CRLYC, CRLNC, CRLQC, CRLDC, CRLEC, and CRLLC (SEQ ID NO: 558 to 579).

Further specific examples of the CXXXC motif are: CXHPC, CHXPC, and CHPXC, wherein X can be any amino acid, more preferably:

CXHPC, such as: CKHPC, CRHPC, CHHPC, CGHPC, CAHPC, CVHPC, CLHPC, CIHPC, CMHPC, CFHPC, CWHPC, CPHPC, CSHPC, CTHPC, CCHPC, CYHPC, CNHPC, CQHPC, CDHPC, CEHPC, and CKHPC (SEQ ID NO: 580 to 601); or

CHXPC, such as: CHKPC, CHRPC, CHHPC, CHGPC, CHAPC, CHVPC, CHLPC, CHIPC, CHMPC, CHFPC, CHWPC, CHPPC, CHSPC, CHTPC, CHCPC, CHYPC, CHNPC, CHQPC, CHDPC, CHEPC, and CHLPC (SEQ ID NO: 602 to 623); or

CHPXC, such as: CHPKC, CHPRC, CHPHC, CHPGC, CHPAC, CHPVC, CHPLC, CHPIC, CHPMC, CHPFC, CHPWC, CHPPC, CHPSC, CHPTC, CHPCC, CHPYC, CHPNC, CHPQC, CHPDC, CHPEC, and CHPLC (SEQ ID NO: 624 to 645).

Any one of these exemplary CXXXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

In a preferred embodiment, said oxidoreductase motif is CX₄C (SEQ ID NO: 646), i.e. CXXXXC, typically CX¹X²X³X⁴C, wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³ and X⁴ in said motif is any amino acid except for C, S, or T. In a specific embodiment, at least one of X¹, X², X³ or X⁴ in said motif is a basic amino acid, such as H, K, or R, or a non-natural basic amino acid as defined herein.

Specific examples of the CXXXXC motif are: CLAVLC, CTVQAC or CGAVHC and their variants such as: CX¹AVLC, CLX²VLC, CLAX³LC, or CLAVX⁴C; CX¹VQAC, CTX²QAC, CTVX³AC, or CTVQX⁴C; CX¹AVHC, CGX²VHC, CGAX³HC, or CGAVX⁴C (SEQ ID NO: 647 to 660); wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein.

Any one of these exemplary CXXXXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

In a preferred embodiment, said oxidoreductase motif is CX₅C (SEQ ID NO: 661), i.e. CXXXXXC, typically CX¹X²X³X⁴X⁵C, wherein X¹, X², X³, X⁴ and X⁵ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³, X⁴ and X⁵ in said motif is any amino acid except for C, S, or T. In a specific embodiment, at least one of X¹, X², X³ X⁴ or X⁵ in said motif is a basic amino acid, such as H, K, or R, or a non-natural basic amino acid as defined herein.

Specific examples of the CXXXXXC motif are: CPAFPLC or CDQGGEC and their variants such as: CX¹AFPLC, CPX²FPLC, CPAX³PLC, CPAFX⁴LC, or CPAFPX⁵C; CX¹QGGEC, CDX²GGEC, CDQX³GEC, CDQGX⁴EC, or CDQGGX⁵C (SEQ ID NO: 662 to 673), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural basic amino acids as defined herein. Any one of these exemplary CXXXXXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

In a preferred embodiment, said oxidoreductase motif is CX₆C (SEQ ID NO: 674), i.e. CXXXXXXC, typically CX¹X²X³X⁴X⁵X⁶C, wherein X¹, X², X³, X⁴ X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³, X⁴, X⁵ and X⁶ in said motif is any amino acid except for C, S, or T. In a specific embodiment, at least one of X¹, X², X³ X⁴, X⁵ or X⁶ in said motif is a basic amino acid, such as H, K, or R, or a non-natural basic amino acid as defined herein.

A specific example of the CXXXXXXC motif is: CDIADKYC or variants thereof such as: CX¹IADKYC, CDX²ADKYC, CDIX³DKYC, CDIAX⁴KYC, CDIADX⁵YC, or CDIADKX⁶C (SEQ ID NO: 675 to 681), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural basic amino acids as defined herein.

Any one of these exemplary CXXXXXXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

Particularly preferred examples of such oxidoreductase motifs are:

C[KHR]C, CX[KHR]XC, CXX[KHR]C, C[KHR]XXC, [KHR]CC, [KHR]CXC, [KHR]XXXC, CC[KHR], CXC[KHR], CXXXC[KHR], [KHR]CC[KHR], [KHR]CXC[KHR], [KHR]CXXXC[KHR], [KHR]C[KHR]C, C[KHR]C[KHR], [KHR]CXX[KHR]C, [KHR]CX[KHR]XC, [KHR]C[KHR]XXC, CXX[KHR]C[KHR], CX[KHR]XC[KHR], C[KHR]XXC[KHR](SEQ ID NO: 682 to 703), and the like.

Further disclosed is a diagnostic in vitro method for detecting class II restricted CD4+ T cells in a sample. In this method a sample is contacted with a complex of an MHC class II molecule and the peptide encoded by the non-immunogenic RNA according to the present invention. The CD4+ T cells are detected by measuring the binding of the complex with cells in the sample, wherein the binding of the complex to a cell is indicative for the presence of CD4+ T cells in the sample. The complex can be a fusion protein of the peptide and an MHC class II molecule. Alternatively, MHC molecules in the complex are tetramers. The complex can be provided as a soluble molecule or can be attached to a carrier.

Further disclosed is a diagnostic in vitro method for detecting NKT cells in a sample. In this method a sample is contacted with a complex of a CD1d molecule and a peptide encoded by the non-immunogenic RNA according to the present invention. The NKT cells are detected by measuring the binding of the complex with cells in the sample, wherein the binding of, complex can be a fusion protein of the peptide and a CD1d molecule.

Accordingly, in particular embodiments, the methods of treatment and prevention of the present invention comprise the administration of non-immunogenic RNA encoding a peptide as described herein, wherein the peptide comprise a T cell epitope of an antigenic protein which plays a role in the disease to be treated (for instance such as those described above). In further particular embodiments, the epitope used is a dominant epitope.

In a preferred embodiment, the non-immunogenic RNA described herein is administered being formulated in carriers or delivery vehicles such as in a nanoparticulate formulation, in particular a lipoplex formulation such as the ones disclosed in WO188730A1. Accordingly, the non-immunogenic RNA molecules described herein may be present formulated in carriers or delivery vehicles such as in nanoparticulates or a nanoparticulate formulation, in particular a lipoplex formulation, as described herein.

In one embodiment, delivery vehicles may be used which deliver the non-immunogenic RNA molecules to antigen presenting cells such as dendrite cells (DCs) in the spleen after systemic administration. For example, nanoparticulate RNA formulations with defined particle size wherein the net charge of the particles is close to zero or negative, such as electro-neutral or negatively charged lipoplexes from RNA and liposomes, e.g. lipoplexes comprising DOTMA and DOPE or DOTMA and Cholesterol, lead to substantial delivery of RNA to spleen DCs after systemic administration. Particularly preferred is a nanoparticulate RNA formulation wherein the charge ratio of positive charges to negative charges in the nanoparticles is 1.4:1 or less and/or the zeta potential of the nanoparticles is 0 or less. In one embodiment, the charge ratio of positive charges to negative charges in the nanoparticles is between 1.4:1 and 1:8, preferably between 1.2:1 and 1:4, e.g. between 1:1 and 1:3 such as between 1:1.2 and 1:2, 1:1.2 and 1:1.8, 1:1.3 and 1:1.7, in particular between 1:1.4 and 1:1.6, such as about 1:1.5. In one embodiment, the zeta potential of the nanoparticles is −5 or less, −10 or less, −15 or less, −20 or less or −25 or less, hi various embodiments, the zeta potential of the nanoparticles is −35 or higher, −30 or higher or −25 or higher. In one embodiment, the nanoparticles have a zeta potential from 0 mV to −50 mV, preferably 0 mV to −40 mV or −10 mV to −30 mV. In one embodiment, the positive charges are contributed by at least one cationic lipid present in the nanoparticles and the negative charges are contributed by the RNA. In one embodiment, the nanoparticles comprise at least one helper lipid. The helper lipid may be a neutral or an anionic lipid.

In one embodiment, the nanoparticles are lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. hi one embodiment, the nanoparticles are lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In one embodiment, the nanoparticles are lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In one embodiment, the nanoparticles are lipoplexes comprising DOTMA and DOPE in a molar ratio of 2:1 to 1:2, preferably 2:1 to 1:1, and wherein the charge ratio of positive charges in DOTMA to negative charges in the RNA is 1.4:1 or less.

In one embodiment, the nanoparticles are lipoplexes comprising DOTMA and cholesterol in a molar ratio of 2:1 to 1:2, preferably 2:1 to 1:1, and wherein the charge ratio of positive charges in DOTMA to negative charges in the RNA is 1.4:1 or less.

In one embodiment, the nanoparticles are lipoplexes comprising DOTAP and DOPE in a molar ratio of 2:1 to 1:2, preferably 2:1 to 1:1, and wherein the charge ratio of positive charges in DOTAP to negative charges in the RNA is 1.4:1 or less.

In one embodiment, the non-immunogenic RNA according to the invention is formulated in F12 or F5 liposomes, preferably F12 liposomes. The term “F12” as used herein designates liposomes comprising DOTMA and DOPE in a molar ratio of 2:1 and lipoplexes with RNA which are formed using such liposomes. The term “F5” as used herein designates liposomes comprising DOTMA and cholesterol in a molar ratio of 1:1 and lipoplexes with RNA which are formed using such liposomes.

As used herein, the term “nanoparticle” refers to any particle having a diameter making the particle suitable for systemic, in particular parenteral, administration, of, in particular, nucleic acids, typically a diameter of less than 1000 nanometers (nm). In some embodiments, a nanoparticle has a diameter of less than 600 nm. In some embodiments, a nanoparticle has a diameter of less than 400 nm. In some embodiments, a nanoparticle has an average diameter in the range of from about SO nm to about 1000 nm, preferably from about 50 nm to about 400 nm, preferably about 100 nm to about 300 nm such as about 150 nm to about 200 nm. In some embodiments, a nanoparticle has a diameter in the range of about 200 to about 700 nm, about 200 to about 600 nm, preferably about 250 to about 550 nm, in particular about 300 to about 500 nm or about 200 to about 400 nm.

As used herein, the term “nanoparticulate formulation” or similar terms refer to any substance that contains at least one nanoparticle. In some embodiments, a nanoparticulate formulation is a uniform collection of nanoparticles. In some embodiments, nanoparticulate formulations are dispersions or emulsions. In general, a dispersion or emulsion is formed when at least two immiscible materials are combined.

The term, “lipoplex” or “nucleic acid lipoplex”, in particular “RNA lipoplex”, when used herein refers to a complex of lipids and nucleic acids, in particular RNA. Lipoplexes are formed spontaneously when cationic liposomes, which often also include a neutral “helper” lipid, are mixed with nucleic acids.

If the present disclosure refers to a charge such as a positive charge, negative charge or neutral charge or a cationic compound, negative compound or neutral compound this generally means that the charge mentioned is present at a selected pH, such as a physiological pH. For example, the term “cationic lipid” means a lipid having a net positive charge at a selected pH, such as a physiological pH. The term “neutral lipid” means a lipid having no net positive or negative charge and can be present in the form of a non-charge or a neutral amphoteric ion at a selected pH, such as a physiological pH. By “physiological pH” herein is meant a pH of about 7.5.

The nanoparticulate carriers such as lipid carriers contemplated for use as disclosed herein can include any substances or vehicles with which nucleic acid such as RNA can be associated, e.g. by forming complexes with the nucleic acid or forming vesicles in which the nucleic acid is enclosed or encapsulated. This may result in increased stability of the nucleic acid compared to naked nucleic acid. In particular, stability of the nucleic acid in blood may be increased. Cationic lipids, cationic polymers and other substances with positive charges may form complexes with negatively charged nucleic acids. These cationic molecules can be used to complex nucleic acids, thereby forming e.g. so-called lipoplexes or polyplexes, respectively, and these complexes have been shown to deliver nucleic acids into cells.

Nanoparticulate nucleic acid preparations for use as disclosed herein can be obtained by various protocols and from various nucleic acid complexing compounds. Lipids, polymers, oligomers, or amphiphiles are typical complexing agents. In one embodiment, the complexing compound comprises at least one agent selected from the group consisting of protamine, polyethyleneimine, a poly-L-lysine, a poly-L-arginine or a histone. Protamine is useful as cationic carrier agent. The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin. According to the invention, the term “protamine” as used herein is meant to comprise any protamine amino acid sequence obtained or derived from native or biological sources including fragments thereof and multimeric forms of said amino acid sequence or fragment thereof. Furthermore, the term encompasses (synthesized) polypeptides which are artificial and specifically designed for specific purposes and cannot be isolated from native or biological sources. The protamine used as disclosed herein can be sulfated protamine or hydrochloride protamine. In a preferred embodiment, the protamine source used for the production of the nanoparticles described herein is protamine 5000 which contains protamine at more than 10 mg/ml (5000 heparin-neutralizing units per ml) in an isotonic salt solution.

Liposomes are microscopic lipidic vesicles often having one or more bilayers of a vesicle-forming lipid, such as a phospholipid, and are capable of encapsulating a drug. Different types of liposomes may be employed in the context of the present disclosure, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bi-layered forms known in the art. The size and lamellarity of the liposome will depend on the manner of preparation and the selection of the type of vesicles to be used will depend on the preferred mode of administration. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state. The described phases may be present in the nanoparticulate nucleic acid formulations disclosed herein.

For formation of nucleic acid lipoplexes from nucleic acid and liposomes, any suitable method of forming liposomes can be used so long as it provides the envisaged nucleic acid lipoplexes. Liposomes may be formed using standard methods such as the reverse evaporation method (REV), the ethanol injection method, the dehydration-rehydration method (DRV), sonication or other suitable methods.

After liposome formation, the liposomes can be sized to obtain a population of liposomes having a substantially homogeneous size range.

Bilayer-forming lipids have typically two hydrocarbon chains, particularly acyl chains, and a head group, either polar or nonpolar. Bilayer-forming lipids are either composed of naturally-occurring lipids or of synthetic origin, including the phospholipids, such as phosphatidylcholine, phosphatidylethanolamine, phosphatide acid, phosphatidylinositol, and sphingomyelin, where the two hydrocarbon chains are typically between about 14-22 carbon atoms in length, and have varying degrees of unsaturation. Other suitable lipids for use in the composition as disclosed herein include glycolipids and sterols such as cholesterol and its various analogs which can also be used in the liposomes.

Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimemymydroxyethyl ammonium (DMRIE), and 2,3-dioleoyloxy-N-[2(sperminecarboxamide)ethyl]-N,N-dimethyl-1-propanamium trifluoroacetate (DOSPA). Preferred are DOTMA, DOTAP, DODAC, and DOSPA. Most preferred is DOTMA.

In addition, the nanoparticles described herein preferably further include a neutral lipid in view of structural stability and the like. The neutral lipid can be appropriately selected in view of the delivery efficiency of the nucleic acid-lipid complex. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, sterol, and cerebroside. Preferred is DOPE and/or DOPC. Most preferred is DOPE. In the case where a cationic liposome includes both a cationic lipid and a neutral lipid, the molar ratio of the cationic lipid to the neutral lipid can be appropriately determined in view of stability of the liposome and the like. According to one embodiment, the nanoparticles described herein may comprise phospholipids. The phospholipids may be a glycerophospholipid. Examples of glycerophospholipid include, without being limited thereto, three types of lipids: (i) zwitterionic phospholipids, which include, for example, phosphatidylcholine (PC), egg yolk phosphatidylcholine, soybean-derived PC in natural, partially hydrogenated or fully hydrogenated form, dimyristoyl phosphatidylcholine (DMPC) sphingomyelin (SM); (ii) negatively charged phospholipids: which include, for example, phosphatidylserine (PS), phosphatidylinositol (PI), phosphatidic acid (PA), phosphatidylglycerol (PG) dipalmipoyl PG, dimyristoyl phosphatidylglycerol (DMPG); synthetic derivatives in which the conjugate renders a zwitterionic phospholipid negatively charged such is the case of methoxy-polyethylene, glycol-distearoyl phosphatidylethanolamine (mPEG-DSPE); and (iii) cationic phospholipids, which include, for example, phosphatidylcholine or sphingomyelin of which the phosphomonoester was O-methylated to form the cationic lipids.

Association of nucleic acid to the lipid carrier can occur, for example, by the nucleic acid filling interstitial spaces of the carrier, such that the carrier physically entraps the nucleic acid, or by covalent, ionic, or hydrogen bonding, or by means of adsorption by non-specific bonds.

In one embodiment, the non-immunogenic RNA that codes for a peptide as described herein comprising an oxidoreductase motif coupled to a T-cell epitope of an autoantigen is administered to a subject. A translation product of the RNA may be formed in cells of the subject and the product may be displayed to the immune system for inducing tolerance to autoreactive T cells targeting the autoantigen.

Alternatively, the present invention envisions embodiments wherein the non-immunogenic RNA expressing a peptide as described herein comprising an oxidoreductase motif coupled to a T-cell epitope of an autoantigen is introduced into cells such as antigen-presenting cells ex vivo, e.g. antigen-presenting cells taken from a patient, and the cells, optionally clonally propagated ex vivo, are transplanted back into the same patient. Transfected cells may be reintroduced into the patient using any means known in the art, preferably in sterile form by intravenous, intracavitary, or intraperitoneal administration. Suitable cells include antigen-presenting cells. The antigen presenting cell preferably is a dendritic cell, a macrophage, a B cell, a mesenchymal stromal cell, an epithelial cell, an endothelial cell and a fibroblastic cell, and most preferably is a dendritic cell. Thus, the invention also includes a method for treating an autoimmune disease, comprising administering to a subject in need isolated antigen-presenting cells that expresses the non-immunogenic RNA described herein. The cells may be autologous, allogeneic, syngeneic or heterologous to the subject. The term “nucleic acid”, as used herein, is intended to include deoxyribonucleic acid (DNA) or ribonucleic acid (RNA) such as cDNA, mRNA, recombinantly produced and chemically synthesized molecules. A nucleic acid may be single-stranded or double-stranded. According to the invention, RNA includes in vitro transcribed RNA (IVT RNA) or synthetic RNA. According to the invention, a nucleic acid is preferably an isolated nucleic acid. Furthermore, the nucleic acids described herein may be recombinant molecules.

The term “isolated nucleic acid” means, when used herein, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e. transfection of, cells, for example, in the form of RNA which can be prepared by in vitro transcription from a DNA template. The RNA can moreover be modified before application by stabilizing sequences, capping, and polyadenylation.

The term “DNA” as used herein relates to a molecule which comprises deoxyribonucleotide residues and preferably is entirely or substantially composed of deoxyribonucleotide residues. “Deoxyribonucleotide” relates to a nucleotide which lacks a hydroxyl group at the 2′-position of a β-D-ribofuranosyl group. The term “DNA” comprises isolated DNA such as partially or completely purified DNA, essentially pure DNA, synthetic DNA, and recombinantly generated DNA and includes modified DNA which differs from naturally occurring DNA by addition, deletion, substitution and/or alteration of one or more nucleotides. Such alterations can include addition of non-nucleotide material, such as to the end(s) of a DNA or internally, for example at one or more nucleotides of the DNA. Nucleotides in DNA molecules can also comprise non-standard nucleotides, such as non-naturally occurring nucleotides or chemically synthesized nucleotides. These altered DNAs can be referred to as analogs or analogs of naturally-occurring DNA.

The term “RNA” as used herein relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. The term includes double stranded RNA, single stranded RNA, isolated RNA such as partially or completely purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA. According to the present invention, the term “RNA” includes and preferably relates to “mRNA” which means “messenger RNA” and relates to a transcript which may be produced using DNA as template and encodes a peptide or protein. mRNA typically comprises a 5′ non translated region (5′-UTR), a protein or peptide coding region and a 3′ non translated region (3′-UTR). mRNA has a limited halftime in cells and in vitro. Preferably, mRNA is produced by in vitro transcription using a DNA template. In one embodiment of the invention, the RNA is obtained by in vitro transcription or chemical synthesis. The in vitro transcription methodology is known to the skilled person. For example, there is a variety of in vitro transcription kits commercially available.

The stability and translation efficiency of RNA may be modified as required. For example, RNA may be stabilized and its translation increased by one or more modifications having a stabilizing effects and/or increasing translation efficiency of RNA. In order to increase expression of the RNA used according to the present invention, it may be modified within the coding region, i.e. the sequence encoding the expressed peptide or protein, preferably without altering the sequence of the expressed peptide or protein, so as to increase the GC-content to increase mRNA stability and to perform a codon optimization and, thus, enhance translation in cells.

The term “modification” in the context of the RNA used herein includes any modification of an RNA which is not naturally present in said RNA. In one embodiment, the RNA does not have uncapped 5′-triphosphates. Removal of such uncapped 5′-triphosphates can be achieved by treating RNA with a phosphatase. The RNA may have modified ribonucleotides in order to increase its stability and/or decrease cytotoxicity. For example, in one embodiment, in the RNA 5-methylcytidine is substituted partially or completely, preferably completely, for cytidine. Alternatively or additionally, in one embodiment, in the RNA pseudouridine is substituted partially or completely, preferably completely, for uridine.

In one embodiment, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap (m7 G). In the context of the present invention, the term “5′-cap” includes a 5′-cap analog that resembles the RNA cap structure and is modified to possess the ability to stabilize RNA and/or enhance translation of RNA if attached thereto, preferably in vivo and/or in a cell.

The RNA may comprise further modifications. For example, a further modification of the RNA used in the present invention may be an extension or truncation of the naturally occurring poly(A) tail or an alteration of the 5′- or 3′-untranslated regions (UTR) such as introduction of a UTR which is not related to the coding region of said RNA, for example, the exchange of the existing 3′-UTR with or the insertion of one or more, preferably two copies of a 3′-UTR derived from a globin gene, such as alpha2-globin, alpha1-globin, beta-globin, preferably beta-globin, more preferably human beta-globin.

RNA having an unmasked poly-A sequence is translated more efficiently than RNA having a masked poly-A sequence. The term “poly(A) tail” or “poly-A sequence” relates to a sequence of adenyl (A) residues which typically is located on the 3′-end of a RNA molecule and “unmasked poly-A sequence” means that the poly-A sequence at the 3′ end of an RNA molecule ends with an A of the poly-A sequence and is not followed by nucleotides other than A located at the 3′ end, i.e. downstream, of the poly-A sequence. Furthermore, a long poly-A sequence of about 120 base pairs results in an optimal transcript stability and translation efficiency of RNA.

Therefore, in order to increase stability and/or expression of the RNA used according to the present invention, it may be modified so as to be present in conjunction with a poly-A sequence, preferably having a length of 10 to 500, more preferably 30 to 300, even more preferably 65 to 200 and especially 100 to 150 adenosine residues. In an especially preferred embodiment the poly-A sequence has a length of approximately 120 adenosine residues. To further increase stability and/or expression of the RNA used according to the invention, the poly-A sequence can be unmasked.

The term “stability” of RNA relates to the “half-life” of RNA. “Half-life” relates to the period of time which is needed to eliminate half of the activity, amount, or number of molecules. In the context of the present invention, the half-life of an RNA is indicative for the stability of said RNA. The half-life of RNA may influence the “duration of expression” of the RNA. It can be expected that RNA having a long half-life will be expressed for an extended time period. Of course, if according to the present invention it is desired to decrease stability and/or translation efficiency of RNA, it is possible to modify RNA so as to interfere with the function of elements as described above increasing the stability and/or translation efficiency of RNA. The RNA to be administered according to the invention is non-immunogenic.

The term “non-immunogenic RNA” as used herein refers to RNA that does not induce a response by the immune system against the RNA molecule itself upon administration, e.g., to a mammal, or induces a weaker response than would have been induced by the same RNA that differs only in that it has not been subjected to the modifications and treatments that render the non-immunogenic RNA non-immunogenic. Said term does however not exclude the indirect immune response elicited toward cytolytic CD4+ T cell development and maturation. In one preferred embodiment non-immunogenic RNA is rendered non-immunogenic by incorporating modified nucleotides suppressing RNA-mediated activation of innate immune receptors into the RNA and removing double-stranded RNA (dsRNA). For rendering the non-immunogenic RNA non-immunogenic by the incorporation of modified nucleotides, any modified nucleotide may be used as long as it lowers or suppresses immunogenicity of the RNA. Particularly preferred are modified nucleotides that suppress RNA-mediated activation of innate immune receptors. In one 10 embodiment, the modified nucleotides comprise a replacement of one or more uridines with a nucleoside comprising a modified nucleobase. In one embodiment, the modified nucleobase is a modified uracil. In one embodiment, the nucleoside comprising a modified nucleobase is selected from the group consisting of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester 20 (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine (τm5s2U), 1-taurinomethyl-4-thio-pseudouridine, 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N(1)-methyl-pseudouridine (m1ψ), 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Urn), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine.

In a preferred embodiment, the structure of a nucleoside comprising a modified nucleobase is N(1)-methylpseudouridine (m1ψ).

During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaselll that specifically hydrolyzes dsRNA but not ssRNA, thereby eliminating dsRNA contaminants from IVT RNA preparations can be used. Furthermore, dsRNA can be separated from ssRNA by using a cellulose material. In one embodiment, an RNA preparation is contacted with a cellulose material and the ssRNA is separated from the cellulose material under conditions which allow binding of dsRNA to the cellulose material and do not allow binding of ssRNA to the cellulose material. As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

Nucleic acids can be transferred into a host cell by physical, chemical or biological means. Physical methods for introducing a nucleic acid into a host cell include calcium phosphate precipitation, lipofection, particle bombardment, microinjection, electroporation, and the like.

Biological methods for introducing a nucleic acid of interest into a host cell include the use of DNA and RNA vectors. Viral vectors, and especially retroviral vectors, have become the most widely used method for inserting genes into mammalian, e.g., human cells. Other viral vectors can be derived from lentivims, poxviruses, herpes simplex virus I, adenoviruses and adeno-associated viruses, and the like. Chemical means for introducing a nucleic acid into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art

“Encoding” refers to the inherent property of specific sequences of nucleotides in a nucleic acid to serve as templates for synthesis of other polymers and macromolecules in biological processes having either a defined sequence of nucleotides or a defined sequence of amino acids. Thus, a nucleic acid encodes a protein if expression (translation and optionally transcription) of the nucleic acid produces the protein in a cell or other biological system. The term “expression” is used according to the invention in its most general meaning and comprises the production of RNA and/or peptides or polypeptides, e.g. by transcription and/or translation. With respect to RNA, the term “expression” or “translation” relates in particular to the production of peptides or polypeptides. It also comprises partial expression of nucleic acids. Moreover, expression can be transient or stable.

In the context of the present invention, the term “transcription” relates to a process, wherein the genetic code in a DNA sequence is transcribed into RNA. Subsequently, the RNA may be translated into protein. According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”. According to the present invention, the RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

The term “translation” according to the invention relates to the process in the ribosomes of a cell by which a strand of messenger RNA directs the assembly of a sequence of amino acids to make a peptide or polypeptide. According to the invention it is preferred that a nucleic acid such as RNA encoding a peptide or protein once taken up by or introduced, i.e. transfected or transduced, into a cell which cell may be present in vitro or in a subject results in expression of said peptide or protein. The cell may express the encoded peptide or protein intracellularly (e.g. in the cytoplasm and/or in the nucleus), may secrete the encoded peptide or protein, or may express it on the surface.

According to the invention, terms such as “nucleic acid expressing” and “nucleic acid encoding” or similar terms are used interchangeably herein and with respect to a particular peptide or polypeptide mean that the nucleic acid, if present in the appropriate environment, preferably within a cell, can be expressed to produce said peptide or polypeptide.

Terms such as “transferring”, “introducing”, “transfecting” or “transducing” are used interchangeably herein and relate to the introduction of nucleic acids, in particular exogenous or heterologous nucleic acids, such as RNA into a cell. According to the present invention, the cell can be present in vitro or in vivo, e.g. the cell can form part of an organ, a tissue and/or an organism. According to the invention, transfection can be transient or stable. For some applications of transfection, it is sufficient if the transfected genetic material is only transiently expressed. Since the nucleic acid introduced in the transfection process is usually not integrated into the nuclear genome, the foreign nucleic acid will be diluted through mitosis or degraded. Cell lines allowing episomal amplification of nucleic acids greatly reduce the rate of dilution. If it is desired that the transfected nucleic acid actually remains in the genome of the cell and its daughter cells, a stable transfection must occur. RNA can be transfected into cells to transiently express its coded protein.

The agents described herein may be administered in the form of any suitable pharmaceutical composition. The term “pharmaceutical composition” relates to a formulation comprising a therapeutically effective agent or a salt thereof, preferably together with pharmaceutical excipients such as buffers, preservatives and tonicity modifiers. Said pharmaceutical composition is useful for treating or preventing a disease or disorder by administration of said pharmaceutical composition to an individual. A pharmaceutical composition is also known in the art as a pharmaceutical formulation. The pharmaceutical composition can be administered locally or systemically.

The term “systemic administration” refers to the administration of a therapeutically effective agent such that the agent becomes widely distributed in the body of an individual in significant amounts and develops a biological effect According to the present invention, it is preferred that administration is by parenteral administration.

The term “parenteral administration” refers to administration of a therapeutically effective agent such that the agent does not pass the intestine. The term “parenteral administration” includes intravenous administration, subcutaneous administration, intramuscular administration, intradermal administration or intraarterial administration but is not limited thereto.

The methods of the invention also preferably comprise further providing to the subject an immune inhibiting compound. If the immune inhibiting compound is a peptide or polypeptide, it can be provided to the subject by administering the immune inhibiting compound or nucleic acid such as RNA encoding the immune inhibiting compound. The immune inhibiting compound can be selected from the group consisting of transforming growth factor beta (TGF-β), interleukin 10 (IL-10), interleukin 1 receptor antagonist (IL-1RA), interleukin 4 (IL-4), interleukin 27 (IL-27), interleukin 35 (TL-35), programmed death-ligand 1 (PD-L1), inducible T-cell co-stimulator ligand (ICOSL), B7-H4, CD39, CD73, FAS, FAS-IL, indoleamine 2,3-dioxygenase 1 (IDOl), indoleamine 2,3-dioxygenase 2 (ID02), acetaldehyde dehydrogenase 1 (ALDHI)/retinaldehyde dehydrogenase (RALDH), arginase 1 (ARG1), arginase 2 (ARG2), nitrous oxide synthase (NOS2), galectin-1, galectin-9, semaphorin 4 A, and any combination thereof. Furthermore, the compositions described herein may comprise such immune inhibiting compound or nucleic acid such as RNA encoding such immune inhibiting compound.

The pharmaceutical composition according to the present invention is generally applied in a “pharmaceutically effective amount” and in “a pharmaceutically acceptable preparation”.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The term “pharmaceutically acceptable” refers to the non-toxicity of a material which does not interact with the action of the active component of the pharmaceutical composition.

The pharmaceutical compositions of the present invention may contain salts, buffers, preserving agents, carriers and optionally other therapeutic agents. Preferably, the pharmaceutical compositions of the present invention comprise one or more pharmaceutically acceptable carriers, diluents and/or excipients.

The term “excipient” is intended to indicate all substances in a pharmaceutical composition which are not active ingredients such as binders, lubricants, thickeners, surface active agents, preservatives, emulsifiers, buffers, flavoring agents, or colorants.

The term “diluent” relates a diluting and/or thinning agent. Moreover, the term “diluent” includes any one or more of fluid, liquid or solid suspension and/or mixing media.

The term “carrier” relates to one or more compatible solid or liquid fillers or diluents, which are suitable for an administration to a human. The term “carrier” relates to a natural or synthetic organic or inorganic component which is combined with an active component in order to facilitate the application of the active component. Preferably, carrier components are sterile liquids such as water or oils, including those which are derived from mineral oil, animals, or plants, such as peanut oil, soy bean oil, sesame oil, sunflower oil, etc. Salt solutions and aqueous dextrose and glycerin solutions may also be used as aqueous carrier compounds.

Pharmaceutically acceptable carriers or diluents for therapeutic use are well known in the pharmaceutical art, and are described, for example, in Remington's Pharmaceutical Sciences, Mack Publishing Co. (A. R Gennaro edit. 1985). Examples of suitable carriers include, for example, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Examples of suitable diluents include ethanol, glycerol and water. Pharmaceutical carriers, excipients or diluents can be selected with regard to the intended route of administration and standard pharmaceutical practice. The pharmaceutical compositions of the present invention may comprise as, or in addition to, the carrier(s), excipient(s) or diluent(s) any suitable binder(s), lubricant(s), suspending agent(s), coating agent(s), and/or solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used. In one embodiment, the composition is an aqueous composition. The aqueous composition may optionally comprise solutes, e.g. salts. In one embodiment, the composition is in the form of a freeze-dried composition. A freeze-dried composition is obtainable by freeze-drying a respective aqueous composition. The present invention is further illustrated by the following figures and examples which are not be construed as limiting the scope of the invention.

Peptides encoded by the non-immunogenic RNA can be tested for the presence of a T cell epitope in in vitro and in vivo methods, and can be tested for their reducing activity in in vitro assays. As a final quality control, the peptides can be tested in in vitro assays to verify whether the peptides can generate CD4+ T cells which are cytolytic, or NKT cells, via an apoptotic pathway for antigen presenting cells presenting the antigen which contains the epitope sequence which is also present in the peptide with the modified oxidoreductase motif. Such peptides disclosed herein can be generated using recombinant DNA techniques, in bacteria, yeast, insect cells, plant cells or mammalian cells. In view of the limited length of the peptides, they can be prepared by chemical peptide synthesis, wherein peptides are prepared by coupling the different amino acids to each other. Chemical synthesis is particularly suitable for the inclusion of e.g. D-amino acids, amino acids with non-naturally occurring side chains or natural amino acids with modified side chains, etc.

Chemical peptide synthesis methods are well described and peptides can be ordered from companies such as Applied Biosystems and other companies.

Peptide synthesis can be performed as either solid phase peptide synthesis (SPPS) or contrary to solution phase peptide synthesis. The best known SPPS methods are t-Boc and Fmoc solid phase chemistry:

During peptide synthesis several protecting groups are used. For example hydroxyl and carboxyl functionalities are protected by t-butyl group, lysine and tryptophan are protected by t-Boc group, and asparagine, glutamine, cysteine and histidine are protected by trityl group, and arginine is protected by the pbf group. If appropriate, such protecting groups can be left on the peptide after synthesis. Peptides can be linked to each other to form longer peptides using a ligation strategy (chemoselective coupling of two unprotected peptide fragments) as originally described by Kent (Schnelzer & Kent (1992) Int. J. Pept. Protein Res. 40, 180-193) and reviewed for example in Tam et al. (2001) Biopolymers 60, 194-205 provides the tremendous potential to achieve protein synthesis which is beyond the scope of SPPS. Many proteins with the size of 100-300 residues have been synthesised successfully by this method. Synthetic peptides have continued to play an ever increasing crucial role in the research fields of biochemistry, pharmacology, neurobiology, enzymology and molecular biology because of the enormous advances in the SPPS.

Alternatively, the peptides can be synthesised by using nucleic acid molecules which encode the peptides of this invention in an appropriate expression vector which include the encoding nucleotide sequences. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridisation methodologies. Such DNA molecules may be incorporated into expression vectors, including plasmids, which are adapted for the expression of the DNA and production of the polypeptide in a suitable host such as bacterium, e.g. Escherichia coli, yeast cell, animal cell or plant cell.

The physical and chemical properties of a peptide of interest (e.g. solubility, stability) are examined to determine whether the peptide is/would be suitable for use in therapeutic compositions. Typically, this is optimized by adjusting the sequence of the peptide. Optionally, the peptide can be modified after synthesis (chemical modifications e.g. adding/deleting functional groups) using techniques known in the art.

The mechanism of action of peptides comprising a standard oxidoreductase motif and an MHC class II T-cell epitope is substantiated with experimental data disclosed in the above cited PCT application WO2008/017517 and publications of the present inventors. The mechanism of action of immunogenic peptides comprising a standard oxidoreductase motif and a CD1d binding NKT-cell epitope is substantiated with experimental data disclosed in the above cited PCT application WO2012/069568 and publications of the present inventors.

The present invention provides methods for generating antigen-specific cytolytic CD4+ T-cells (when using an immunogenic peptide as disclosed herein comprising an MHC class II epitope), or antigen-specific cytolytic NKT-cells (when using an immunogenic peptide as disclosed herein comprising an NKT cell epitope binding the CD1d molecule) either in vivo or in vitro, by administering non immunogenic RNA encoding a peptide comprising a T-cell epitope (MHC class II or NKT epitope respectively) coupled to an oxidoreductase motif as described herein.

The present invention describes in vivo methods for the production of the antigen-specific CD4+ T cells or NKT cells. A particular embodiment relates to the method for producing or isolating the CD4+ T cells or NKT cells by immunising animals (including humans) with the non-immunogenic RNA encoding peptides as disclosed herein and then isolating the CD4+ T cells or NKT cells from the immunised animals. The present invention describes in vitro methods for the production of antigen specific cytolytic CD4+ T cells or NKT cells towards APC. The present invention provides methods for generating antigen specific cytolytic CD4+ T cells and NKT cells towards APC.

In one embodiment, methods are provided which comprise the isolation of peripheral blood cells, the stimulation of the cell population in vitro by a peptide as described herein and the expansion of the stimulated cell population, more particularly in the presence of IL-2. The methods according to the invention have the advantage a high number of CD4+ T cells is produced and that the CD4+ T cells can be generated which are specific for the antigenic protein (by using a peptide comprising an antigen-specific epitope).

In an alternative embodiment, the CD4+ T cells can be generated in vivo, i.e. by the injection of the immunogenic peptides or non-immunogenic RNA encoding such peptides as described herein to a subject, and collection of the cytolytic CD4+ T cells generated in vivo.

The antigen-specific cytolytic CD4+ T cells towards APC, obtainable by the methods of the present invention are of particular interest for the administration to mammals for immunotherapy, in the prevention of allergic reactions and the treatment of auto-immune diseases. Both the use of allogenic and autogeneic cells are envisaged. Cytolytic CD4+ T cells populations are obtained as described herein below.

In one embodiment, the invention provides ways to expand specific NKT cells, with as a consequence increased activity comprising, but not limited to:
(i) increased cytokine production
(ii) increased contact- and soluble factor-dependent elimination of antigen-presenting cells. The result is therefore a more efficient response towards intracellular pathogens, autoantigens, preferably autoantigens involved in type-1 diabetes (T1D), demyelinating disorders such as multiple sclerosis (MS) or neuromyelitis optica (NMO), or rheumatoid arthritis (RA).

The present invention also relates to the identification of NKT cells with required properties in body fluids or organs. The method comprises identification of NKT cells by virtue of their surface phenotype, including expression of NK1.1, CD4, NKG2D and CD244. Cells are then contacted with NKT cell epitopes defined as peptides able to be presented by the CD1d molecule. Cells are then expanded in vitro in the presence of IL-2 or IL-15 or IL-7.

Antigen-specific cytolytic CD4+ T cells or NKT cells as described herein can be used as a medicament, more particularly for use in adoptive cell therapy, more particularly in the treatment of acute allergic reactions and relapses of autoimmune diseases such as in type-1 diabetes (T1D), demyelinating disorders such as multiple sclerosis (MS) or neuromyelitis optica (NMO), or rheumatoid arthritis (RA). Isolated cytolytic CD4+ T cells or NKT cells or cell populations, more particularly antigen-specific cytolytic CD4+ T cell or NKT cell populations generated as described are used for the manufacture of a medicament for the prevention or treatment of (auto-)immune disorders. Methods of treatment by using the isolated or generated cytolytic CD4+ T cells or NKT cells are disclosed.

As explained in WO2008/017517 cytolytic CD4+ T cells towards APC can be distinguished from natural Treg cells based on expression characteristics of the cells. More particularly, a cytolytic CD4+ T cell population demonstrates one or more of the following characteristics compared to a natural Treg cell population:
an increased expression of surface markers including CD103, CTLA-4, Fasl and ICOS upon activation, intermediate expression of CD25, expression of CD4, ICOS, CTLA-4, GITR and low or no expression of CD127 (IL7-R), no expression of CD27, expression of transcription factor T-bet and egr-2 (Krox-20) but not of the transcription repressor Foxp3, a high production of IFN-gamma and no or only trace amounts of IL-10, IL-4, IL-5, IL-13 or TGF-beta.

Further the cytolytic T cells express CD45RO and/or CD45RA, do not express CCR7, CD27 and present high levels of granzyme B and other granzymes as well as Fas ligand.

As explained in WO2008/017517 cytolytic NKT cells against towards APC can be distinguished from non-cytolytic NKT cells based on expression characteristics of the cells. More particularly, a cytolytic CD4+NKT cell population demonstrates one or more of the following characteristics compared to a non-cytolytic NKT cell population: expression of NK1.I, CD4, NKG2D and CD244.

The peptides or non-immunogenic RNA encoding such peptides as described herein will, upon administration to a living animal, typically a human being, elicit specific T cells exerting a suppressive activity on bystander T cells.

In specific embodiments the cytolytic cell populations of the present invention are characterised by the expression of FasL and/or Interferon gamma. In specific embodiments the cytolytic cell populations of the present invention are further characterised by the expression of GranzymeB.

This mechanism also implies and the experimental results show that the peptides encoded by the non-immunogenic RNA as described herein, although comprising a specific T-cell epitope of a certain antigen, can be used for the prevention or treatment of disorders elicited by an immune reaction against other T-cell epitopes of the same antigen or in certain circumstances even for the treatment of disorders elicited by an immune reaction against other T-cell epitopes of other different antigens if they would be presented through the same mechanism by MHC class II molecules or CD1d molecules in the vicinity of T cells activated by said peptides (bystander effect).

Isolated cell populations of the cell type having the characteristics described above, which, in addition are antigen-specific, i.e. capable of suppressing an antigen-specific immune response are disclosed.

The present invention provides pharmaceutical compositions comprising one or more non-immunogenic RNA species encoding one or more peptides according to the present invention, further comprising a pharmaceutically acceptable carrier. As detailed above, the present invention also relates to the compositions for use as a medicine or to methods of treating a mammal of an immune disorder by using the composition and to the use of the compositions for the manufacture of a medicament for the prevention or treatment of immune disorders. The pharmaceutical composition could for example be a vaccine suitable for treating or preventing (auto)immune disorders, especially airborne and foodborne allergy, as well as diseases of allergic origin.

While it is possible for the active ingredients to be administered alone, they typically are presented as pharmaceutical formulations. The formulations, both for veterinary and for human use as described herein comprise at least one active ingredient, as above described, together with one or more pharmaceutically acceptable carriers. The present invention relates to pharmaceutical compositions, comprising, as an active ingredient, non-immunogenic RNA species encoding one or more peptides as described herein, in admixture with a pharmaceutically acceptable carrier. The pharmaceutical composition of the present invention should comprise a therapeutically effective amount of the active ingredient, such as indicated hereinafter in respect to the method of treatment or prevention. Optionally, the composition further comprises other therapeutic ingredients. Suitable other therapeutic ingredients, as well as their usual dosage depending on the class to which they belong, are well known to those skilled in the art and can be selected from other known drugs used to treat (auto-)immune disorders.

The non-immunogenic RNA according to the invention can be administered through different routes including regional, systemic, oral (solid form or inhalation), rectal, nasal, topical (including ocular, buccal and sublingual), vaginal and parenteral (including subcutaneous, intramuscular, intravenous, intradermal, intra-arterial, intrathecal and epidural). The preferred route of administration may vary with for example the condition of the recipient or with the diseases to be treated. As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration.

The non-immunogenic RNA that encodes peptides as described herein comprising an MHC class II T-cell epitope will be delivered to (immature) dendritic cells that will present the peptide at their surface, becoming an APC. These cells will induce cytolytic CD4+ T cells that (1) induce APC apoptosis after MHC-class II dependent cognate activation, affecting both dendritic and B cells, as demonstrated in vitro and in vivo, and (2) suppress bystander T cells by a contact-dependent mechanism. Cytolytic CD4+ T cells can be distinguished from both natural and adaptive Tregs, as discussed in detail in WO2008/017517.

The non-immunogenic RNA that encodes peptides as described herein containing hydrophobic residues that confer the capacity to bind to the CD1d molecule. Upon administration, the RNA is taken up by (immature) dendritic cells and directed to the late endosome where they are loaded onto CD1d and presented at the surface of the APC. Once presented by CD1d molecule, the oxidoreductase motif in the encoded peptides enhances the capacity to activate NKT cells, becoming cytolytic NKT cells. Said encoded peptides activate the production of cytokine, such as IFN-gamma, which will activate other effector cells including CD4+ T cells and CD8+ T cells. Both CD4+ and CD8+ T cells can participate in the elimination of the cell presenting the antigen as discussed in detail in WO2012/069568.

The present invention will now be illustrated by means of the following examples which are provided without any limiting intention. Furthermore, all references described herein are explicitly included herein by reference.

### EXAMPLES

### Example 1: Materials and Methods

### Flow Cytometric Analysis

Fluorescence-activated cell sorting (FACS) surface and intracellular antibodies can be purchased from eBioscience or BD Pharmingen and used in accordance with the manufacturer's protocol. Antibodies used can be the following: CD11b, CD11c, CD19, CD4, CD40L, CD49b, CD69, CD8, CD86, CD90.1, IFNγ and IL-17A. Single cell suspensions from different organs are stained for 30 min at 4° C. for extracellular markers. For intracellular cytokine staining of IFNγ, 1L-17A and CD40L, cells are isolated as described and additionally stimulated in culture medium containing MOG35-55 peptide (conc. 15 μg/ml), Monensin (GolgiStop, BD-Bioscience) and Brefeldin A at 37° C., 5% CO2 for 6 h. After performing live-dead staining (viability dye eFluor® 506, eBioscience) and staining of cell-surface markers, cells are fixed and permeabilized using Cytofix/Cytoperm and Perm/Wash buffer from BD Biosciences in accordance with the manufacturer's protocol. Cells are incubated for 30 min at 4° C. with intracellular antibodies and washed twice in Perm/Wash before FACS analysis. Samples are acquired with FACS Canto II and analyzed by using FlowJo (Tree Star) software.

### In Vivo Bioluminescence Imaging (BLI)

Translation efficiency of non-immunogenic mRNA in spleen cells is investigated using Xenogen IVIS Spectrum in vivo imaging system (Caliper Life Sciences). 6 h, 24 h, 48 h and 72 h after RNA-LPX-immunization of non-immunogenic LUC-mRNA, mice are injected i.p. with an aqueous solution of D-luciferin (250 μl, 1.6 mg in PBS) (BD Biosciences). After 5 minutes, signal intensities from the spleen are defined and measured by in vivo bioluminescence in regions of interest (ROI) and quantified as total flux (photons/sec) using IVIS Living Image 4.0 Software. The acquisition time is 1 min at binning 4. During bioluminescent imaging of the live mice, mice were anesthetized with a dose of 2.5% isoflurane/oxygen mixture. The LUC signal intensity of emitted photons of live animals is depicted as a grayscale image, where black is the least intense and white the most intense bioluminescence signal. Images of mice are analyzed using the IVIS Living Image Software.

### Magnetic Activated Cell Sorting (MACS)

CD4+ T cells can be isolated from mice spleen and lymph node by positive selection using MACS (Miltenyi Biotec) according to the manufacturer's instruction. CD4+ T cells are purified using CD4 (L3T4) Micro Beads.

### Generation of Bone Marrow-Derived DCs (BMDCs)

Bone marrow (BM) cells are extracted from femur and tibia of the mice and cultured in tissue culture flasks at 37° C. in RPMI 1640+ GlutaMAX-I medium (Gibco) supplemented with 10% FBS (Biochrom), 1% Sodium Pyruvate 100× (Gibco), 1% MEM NEAA 100× (Gibco), 0.5% penicillin-streptomycin (Gibco), 50 μM 2-Mercaptoethanol (Life Technologies) and 1000 U/ml dendritic cell (DC) differentiation factor granulocyte-macrophage colony-stimulation factor (GM-CSF) (Peprotech). At day 6, 2×106 DCs/ml are pre-incubated for 3 h at 37° C. with MOG35-55 peptide (15 μg/ml) and then co-cultured with T cells in a total volume of 200 μl medium in a 96-well plate.

### Adoptive T Cell Transfer and In Vivo Proliferation Assay

For proliferation studies, enriched CD4+ T cells obtained from mice can be CTV-labeled (CellTrace Violet cell proliferation kit, Thermo Fisher Scientific) according to the manufacturer's instruction, counted and 7×106 cells injected i.v. into the retro-orbital plexus in 200 μl PBS into naive mice recipient mice under anesthesia. At the next day mice are immunized with 10, 20 or 40 μg of non-immunogenic mRNA coding for the wt MOG35-55 epitope or IMCY-0189 (see sequences below). Control mice receive either 20 μg of non-immunogenic irrelevant mRNA or saline. 4 days after T cell transfer, mice are sacrificed and cells are analyzed for proliferation by flow cytometry.

### ELISA

Detection of mouse IFN-a can be performed by ELISA (PBL) 6 hours after RNA-immunization in mouse sera using standard ELISA assay according to manufacturer's instructions.

### Isolation of Splenic, LN and CNS Infiltrates

All cell-based analyses can be performed on single cell suspension of spleen, lymph node and CNS organs. In brief, spleens and LNs are incubated with 1 mg/ml collagenase D and 0.1 mg/ml DNasel for 15 min and then mashed through a 70 μm cell strainer while rinsing with PBS. Erythrocyte lysis (hypotonic lysis buffer: 1 g KHCO3 and 8.25 g NH4CI dissolved in 1 L H20 and 200 μl 0.5 M EDTA) is performed on single cell suspension of splenocytes. After an additional washing step with PBS, cells are counted with the Vi-Cell cell counter.

For isolation of CNS infiltrates from brain and spinal cord, mice are anesthetized with Ketamine-Rompun and perfused using 0.9% NaCl through the heart left ventricle. The brain and spinal cord are removed manually, cut into small pieces and digested in PBS++ (PBS with calcium and magnesium, Gibco) containing collagenase D (1 mg/ml) and DNase I (0.1 mg/ml) for 20 min at 37° C. The digested tissue is then homogenized manually by sucking up the tissue pieces into a syringe and pressing out again against the wall of a 15 ml falcon tube. This is performed up to 10 times, until no pieces were visible. The single cell suspension is resuspended in 70% Percoll and layered under a 30:37 Percoll gradient. The final Percoll gradient is 30:37:70% and is centrifuged at 300 g for 40 min at room temperature. The mononuclear cell layer lying at the interphase between 70% and 37% Percoll is washed with 2% FCS/PBS before further analyses.

### Example 2: Examination of Non-Immunogenic mRNA Regarding Activation of Splenocytes

In order to analyze the activation of splenocytes and translation efficiency of the used non-immunogenic mRNA, mice can be injected intravenously into the retro-orbital plexus with Luciferase-mRNA (10 μg) complexed with F12 liposomes as described above. Luciferase activities are assessed via in vivo imaging e.g. 6, 24, 48 and 72 hours after RNA-LPX injection. Immunization of mice with non-immunogenic mRNA should lead to a lasting high translation of the LUC-mRNA. Consequently, LUC protein expression can generally be detected up to 72 hours after mRNA immunization.

Furthermore, immunization of mice with non-immunogenic mRNA should lead to no upregulation of activation markers CD86 on DCs and CD69 on lymphocytes like in untreated control mice. In mice immunized with non-immunogenic LUC-mRNA also no IFNa should be detected in the blood 6 hours after mRNA immunization.

### Example 3: Characterization of Non-Immunogenic mRNA

Non-immunogenic mRNA is used to deliver specific disease relevant antigens to dendritic cells to ensure antigen-presentation without immune activation in therapeutic applications. It has been shown that the incorporation of N(1)-methylpseudouridine (m1ψ) into mRNA enhances the protein expression in the cells and reduces the immunogenicity of the mRNA in mammalian cell lines as well as in vivo in mice (Andries et al., 2015, J Control Release. 217, 337-344). This effect relies most likely on the increased ability of the mRNA to evade activation of endosomal Toll-like receptor 3 (TLR3) and downstream innate immune signaling (Andries et al., 2015). Additionally, to the use of N(1)-methylpseudouridine (m1ψ) instead of the nucleoside uridine for the incorporation into the mRNA during in vitro transcription, HPLC purification of the synthetic mRNA can also be performed. It eliminates furthermore immune activation and improves the translation of the nucleoside-modified, protein-encoding mRNA (Kariko et al., 2011, Nucleic Acids Res. 39, el42). By HPLC purification remaining double-stranded mRNA contaminants are removed after mRNA in vitro transcription, resulting in mRNA that does not induce Interferon signaling and inflammatory cytokines (Kariko et al., 2011). An alternative method for the purification of the nucleoside-purified mRNA is the Cellulose-purification (PCT/EP20167059056).

To investigate whether the mRNA used for therapeutic application is really non-immunogenic, Bioanalyzer and Dotblot analysis can be performed to ensure the integrity and the purity of the mRNA.

Dot-Blot-Analysis for Quality Control of mRNA

In vitro transcribed, modified and HPLC-purified mRNA is analyzed for double-stranded mRNA (dsRNA) by Dot-blot. 1 μg of different mRNA constructs is loaded on a NYTRAN SPC membrane (GE Healthcare), blocked and then incubated with J2 antibody (SCICONS English and Scientific Consulting) for the detection of dsRNA. As secondary antibody 10 anti-mouse HRP antibody (Jackson ImmunoResearch) is used and membranes are analyzed with a BioRad ChemiDoc.

### Example 4: Design of Immunogenic Peptides with an Oxidoreductase Motif Comprising a Different Number of Amino Acids in Between the 2 Cysteines (C—X_{N}—C) and Non-Immunogenic RNA Encoding Such

IMCY-0443 and IMCY-0189 peptides were designed (table 1). They comprise an oxidoreductase motif (H)CPYC (SEQ ID NO 34 and 35), a linker GW, a murine Myelin 20 Oligodendrocyte Glycoprotein (MOG) MHCII T cell epitope YRSPFSRW (SEQ ID NO: 36) and a flanking sequence HLYR (SEQ ID NO: 705). Variants of IMCY-0443 and IMCY-0189 were also designed in a way that the sequences were modified to have a different number of amino acids between the 2 cysteines of the oxidoreductase motif (table 1). Basic amino acids (K or R) were added at the N-terminus or within the oxidoreductase motif of the variants. Control peptides with alanine instead of cysteine residues were also synthesized (so-called AA controls). IMCY-0017 (which corresponds to wt MOG₃₅₋₅₅ peptide comprising the MHCII T cell epitope YRSPFSRW-SEQ ID NO: 704), IMCY-0069 (peptide comprising a classical oxidoreductase motif HCPYC (SEQ ID NO: 24) and a modified murine pre-proinsulin epitope) and IMCY-0257 (peptide comprising a classical oxidoreductase motif HCPYC (SEQ ID NO: 24) and a DBY antigen epitope) were also designed as controls (table 1).

TABLE 1
immunogenic peptides with an oxidoreductase motif comprising
different number of amino acids in between the 2 cysteines (C-X_{N}-C).

| IMCY-No | SEQUENCE | SEQ ID # |
|---|---|---|
| IMCY-0443 | CPYC-GWYRSPFSRVVHLYR-NH₂ | 706 |
| IMCY-0189 | HCPYC-GWYRSPFSRVVHLYR-NH₂ | 707 |
| IMCY-0190 | HAPYA-GWYRSPFSRVVHLYR-NH₂ | 708 |
| IMCY-0451 | KCC-GWYRSPFSRVVHLYR-NH₂ | 709 |
| IMCY-0511 | KAA-GWYRSPFSRVVHLYR-NH₂ | 710 |
| IMCY-0452 | CC-GWYRSPFSRVVHLYR-NH₂ | 711 |
| IMCY-0453 | KCRC-GWYRSPFSRVVHLYR-NH₂ | 712 |
| IMCY-0513 | KARA-GWYRSPFSRVVHLYR-NH₂ | 713 |
| IMCY-0454 | CRC-GWYRSPFSRVVHLYR-NH₂ | 714 |
| IMCY-0512 | ARA-GWYRSPFSRVVHLYR-NH₂ | 715 |
| IMCY-0455 | KCRPYC-GWYRSPFSRVVHLYR-NH₂ | 716 |
| IMCY-0515 | KARPYA-GWYRSPFSRVVHLYR-NH₂ | 717 |
| IMCY-0456 | CRPYC-GWYRSPFSRVVHLYR-NH₂ | 718 |
| IMCY-0514 | ARPYA-GWYRSPFSRVVHLYR-NH₂ | 719 |
| IMCY-0017 (wt MOG₃₅₋₅₅) | MEVGWYRSPFSRVVHLYRNGK-NH₂ | 720 |
| IMCY-0257 | HCPYC-NGGFNSNRANSSRSS-NH₂ | 721 |
| IMCY-0069 | HCPYC-LALWEPKPTQAFVK-NH₂ | 722 |

Generation of Non-Immunogenic mRNA-Encoding Peptides

Non-immunogenic mRNA comprising N(1)-methyl-pseudouridine (m1ψ) instead of uridine (abbreviated m1Ψ mRNA) encoding IMCY-0189 and IMCY-0017 (wt MOG₃₅₋₅₅) were generated as follows.

### Dna Template Generation for In Vitro Transcription

DNA templates for in vitro transcription were generated by insertion of the following coding sequences into pmRVac vector using Gibson Assembly method:

```
for IMCY-0189 m1Ψ mRNA
(SEQ ID NO: 768-DNA sequence):
ATGCACTGTCCCTATTGCGGTTGGTACCGTTCTCCCTTCTCAAGAGTG
  M H C P Y C G W Y R S P F S R V
GTTCACCTCTACCGA
  V H L Y R
encoding the peptide: M-HCPYC-GWYRSPFSRVVHLYR
(SEQ ID NO: 767)
for MOG₃₅₋₅₅ m1Ψ mRNA
(SEQ ID NO: 769-DNA sequence):
ATGGAGGTGGGTTGGTACCGTTCTCCCTTCTCAAGAGTGGTTCACCTC
  M E V G W Y R S P F S R V V H L
TACCGAAATGGCAAG
  Y R N G K
encoding the peptide: MEVGWYRSPFSRVVHLYRNGK
(SEQ ID NO: 720)
for IMCY-0098 m1Ψ mRNA
(SEQ ID NO: 790-DNA sequence):
ATGCACTGTCCCTATTGCAGCCTGCAGCCCTTGGCCCTGGAGGGGTCC
  M H C P Y C S L Q P L A L E G S
CTGCAGAAGCGTGGC
  L Q K R G
endcoding the peptide: M-HCPY-CSLQPLALEGSLQKRG
(SEQ ID NO: 791)
for IMCY-0141 m1Ψ mRNA
(SEQ ID NO: 792-DNA sequence):
ATGAAGCACTGTCCCTATTGCGTGAGGTACTTTCTGAGGGTGCCCAGC
  M K H C P Y C V R Y F L R V P S
TGGAAGATAACCCTGTTTAAAAAG
  W K T I L F K K
encoding the peptide: M-KHCPYC-VRYFLRVPSWKTILFKK
(SEQ ID NO: 793)
```

The pmRVac vector contains a T7 promoter sequence, a 5′ UTR with a Kozak sequence, a 3′ UTR, a 110 nt segmented polyA and a SapI restriction enzyme site used for run-off transcription. The insertion site was in between the UTRs. The Gibson Assembly reaction product was transformed into E. coli and plated on LB containing kanamycin. The resulting colonies were screened by PCR to identify positive insertion events. The PCR positive clones were evaluated further by restriction digestion and Sanger sequencing. The correct plasmid was cultured in LB media and purified using an endotoxin-free plasmid midiprep kit. Plasmid DNA concentration and purity were measured on a UV-Vis spectrophotometer, and then linearized using SapI restriction enzyme.

### Mrna Synthesis

Cap1 mRNA was generated by T7 in vitro transcription method and subsequent enzymatic capping and methylation. To produce uncapped in vitro RNA transcripts, linear DNA template was mixed with T7 RNA polymerase, nucleotide triphosphates (NTPs) and magnesium-containing buffer to set up the in vitro transcription reaction. The reaction mixture was incubated for 2 hours at 37° C. N(1)-methyl-pseudouridine (m1Ψ) was used to replace UTP in vitro transcription.

Cap1 mRNA was produced using vaccinia capping system. After denaturation of uncapped transcripts by heating, vaccinia capping enzyme, 2′-0 methyltransferase, GTP, S-adenosyl methionine (SAM) and capping buffer were added to set up the capping reaction. The reaction mixture was incubated for 1 hour at 37° C. Subsequently, template DNA was removed by DNase I treatment.

Capped transcripts were further purified using magnetic beads. The isolated mRNA was eluted with an acidic buffer and stored at −80° C. mRNA concentration and purity were measured on a UV-Vis spectrophotometer. mRNA integrity was measured by denaturing agarose gel analysis. mRNA sequencing was used to identify mRNA molecules.

mRNA can also be further purified by HPLC or Cellulose treatment. For HPLC purification the protocol of Weissman et al., 2013 (Methods Mol Bio. 969, 43-43), is adapted and elution of the mRNA is performed with a gradient of 38%-70% of Buffer B. For all generated mRNAs quality controls were performed (Bioanalyser and Dot-Blot analysis) to ensure the purity and integrity of the mRNA.

Encapsulation of mRNA in Lipid Nanoparticles (LNP)

Lipids (ionizable cationic lipid SM-102, helper lipid DSPC, cholesterol and PEGylated lipid PEG2000-DMG) were dissolved in ethanol at an optimal molar ratio. The mRNA was diluted in an acidification buffer of sodium citrate to a desired concentration. LNPs were produced by mixing lipids with mRNA using a microfluidic mixer. Subsequently, LNPs were dialyzed against Tris-HCl buffer with sucrose in a dialysis cassette.

After dialysis, LNPs were passed through a 0.22 μm filter and stored at −80° C. RiboGreen assay was used to quantify mRNA in LNPs. Particle size and surface charge were determined by dynamic light scattering and zeta potential using a Zetasizer device. Endotoxin level was measured by Kinetic chromogenic TAL assay.

The LNP dispersion was prepared in 20 mM Tris, 87 mg/mL sucrose, 10.7 mM sodium acetate, pH 7.5.

### Example 5: Effect of the Administration of Non-Immunogenic RNA Encoding Peptides on Experimental Auto-Immune Encephalomyelitis (EAE) Development in Mice

### Groups of Mice and Dosing

C57BL/6 female mice (The Jackson Laboratory, 9 weeks old on Day 0) were used. Mice 15 were acclimated for 7 days prior to the start of the study. They were assigned to 3 groups (16 mice per group) according to Table 2. The distribution of the mice was performed in a balanced manner to achieve similar average weight across the groups at the start of the study.

TABLE 2
Treatment regimen

| | # | | | | | Dosing | |
|---|---|---|---|---|---|---|---|
| Group | animals | Treatment | Vehicle | Dose | Route | days | Purpose |
| 1 | 16 | Vehicle (Tris | 20 mM Tris | — | i.v. | 7, 10 | Negative |
| | | buffer) | buffer | | | | control |
| 2 | 16 | MOG₃₅₋₅₅ | 20 mM Tris | 10.2 μg (Day | i.v. | 7, 10 | Test |
| | | m1Ψ | buffer | 7), 4.8 μg | | | |
| | | mRNA/LNP | | (Day 10) | | | |
| 3 | 16 | IMCY-0189 | 20 mM Tris | 10.2 μg (Day | i.v. | 7, 10 | Test |
| | | m1Ψ | buffer | 7), 4.8 μg | | | |
| | | mRNA/LNP | | (Day 10) | | | |

### Eae Induction in Mice

EAE was induced in all mice as follows:
Day 0, Hour 0: Immunization with MOG₃₅₋₅₅/CFA
Day 0, Hour 2: Injection of pertussis toxin
Day 1, Hour 0: 2^{nd} injection of pertussis toxin (24 hours after initial immunization)

Mice were injected subcutaneously at 2 sites in the back with the emulsion component (containing MOG₃₅₋₅₅) of Hooke Kit™ MOG₃₅₋₅₅/CFA Emulsion PTX, catalog number EK-2110 (Hooke Laboratories, Lawrence MA). The first site of injection was in the upper back, approximately 1 cm caudal of the neckline. The second site was in the lower back, approximately 2 cm cranial of the base of the tail. The injection volume was 0.1 mL at each site.

Within 2 hours of the injection of emulsion, and then again 24 hours after the injection of emulsion, the pertussis toxin component of the kit was administered intraperitoneally.

### Rna Preparation and Treatment

To determine protective immunity in the EAE model, mice were treated at day 7 and day 10 after disease induction with 10.2 (day 7) or 4.8 (day 10) μg of non-immunogenic LNP-encapsulated m1Ψ RNA encoding the MOG₃₅₋₅₅ (IMCY-0017) or IMCY-0189 peptides displayed in table 1. mRNA was injected i.v. into the tail vein under anesthesia using an oxygen-isoflurane vaporizer (2.5% Isoflurane).

### Group 1: Vehicle (Tris Buffer)

### Preparation for 25 Mice (Injection Of16 Mice)

2000 μl of 20 mM Tris buffer was prepared and was injected intravenously into tail vein (1×85 μL/mouse on Day 7, 1×40 μL/mouse on Day 10).

Group 2: MOG₃₅₋₅₅ mRNA/LNP (MOG35-55_m1ψ)

### Preparation for 25 Mice (Injection of 16 Mice)

Vials of LNP/mRNA solution were thawed at room temperature for 10 minutes, vortexed 5 sec, and 4 vials were pooled to obtain 2000 μL of a stock at 120 μg/mL. mRNAs were then injected intravenously into tail vein (1×85 μL/mouse on Day 7, 1×40 μL/mouse 5 on Day 10).

Group 3: IMCY-0189 mRNA/LNP (IMCY-0189_m1t1i)

### Preparation for 25 Mice (Injection Of16 Mice)

Vials of LNP/mRNA solution were thawed at room temperature for 10 minutes, vortexed 5 sec, and 4 vials were pooled to obtain 2000 μL of a stock at 120 μg/mL. mRNAs were 10 then injected intravenously into tail vein (1×85 μL/mouse on Day 7, 1×40 μL/mouse on Day 10).

### Readouts

The EAE scores were used as readout. From day 7 until the end of the study, the animals are scored daily. The person who performed the scoring is unaware both of treatment and of the previous score of each mouse (blinded scoring). EAE is scored on the scale 0 to 5. In-between scores are assigned when the clinical signs fall between the two below defined scores.

TABLE 3
EAE scoring criteria

| Score | Clinical observations |
|---|---|
| 0 | No obvious changes in motor functions of the mouse. |
| | When picked up by the tail, the tail has tension and is erect. |
| | Hind legs are usually spread apart. When the mouse is walking, |
| | no gait or head tilting can be observed. |
| 1 | Limp tail. |
| | When the mouse is picked up by the tail, instead of being erect, |
| | the whole tail drapes over the finger of the operator. |
| 2 | Limp tail and weakness of hind legs. |
| | When the mouse is picked up by the tail, legs are not spread |
| | apart but held closely together. Furthermore, it displays clearly |
| | apparent wobbly walk. |
| 3 | Limp tail and complete paralysis of hind legs (most common), |
| | OR |
| | Limp tail with paralysis of one front and one hind leg, |
| | OR |
| | All the following: |
| | Severe head tilting, |
| | Walking only along the edges of the cage, |
| | Pushing against the cage wall, |
| | Spinning when picked up by the tail. |
| 4 | Limp tail, complete paralysis of the hind legs and partial |
| | paralysis of the front legs. |
| | Mouse is minimally moving around the cage but is alert and |
| | feeding. Euthanasia is recommended if the mouse scores 4 for 2 |
| | days in a row. |
| 5 | After being euthanized, a mouse is scored 5 for the rest of the |
| | experiment. |

### Statistical Analysis

Area under the curve (AUC) and Mean Maximal Score (MMS) data were analyzed by performing Unpaired t tests comparing the treated groups to the vehicle group respectively. Significant differences are referred as follows: *p<0.05, **p<0.01.

### Results

### EAE Scoring

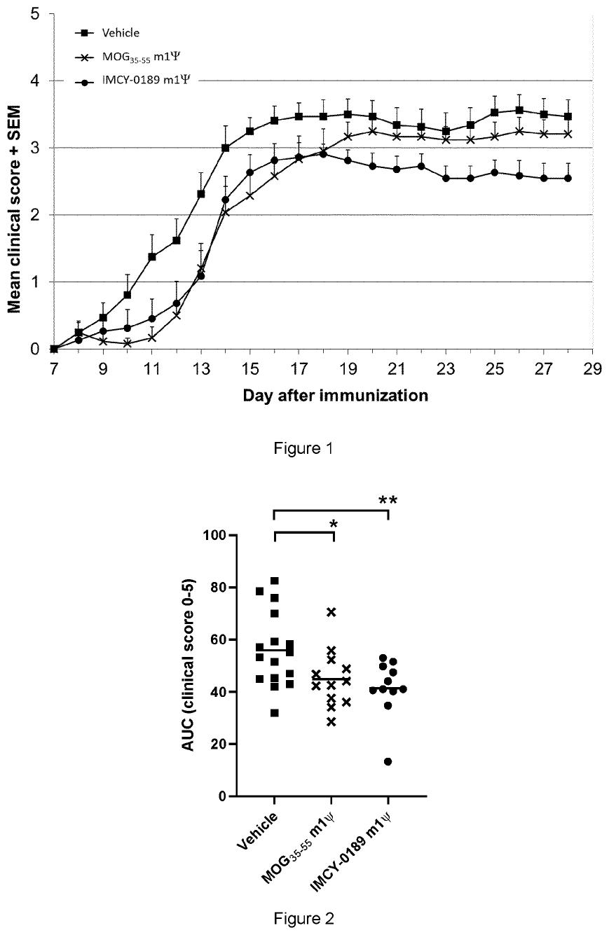

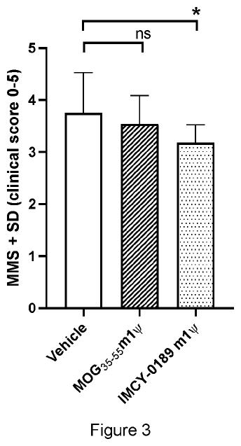

EAE development was evaluated by comparing clinical EAE readouts for all groups to the vehicle group. EAE scoring, AUC (area under the curve) and MMS (mean maximal score) are presented in FIGS. 1, 2 and 3.

Mice of the vehicle group (negative control) developed EAE within the expected range for this model. Four (4) mice died in this group due to severe EAE.

Mice treated either with IMCY-0189 m1Ψ or with MOG₃₅₋₅₅ m1Ψ both showed postponed disease onset (FIG. 1) and statistically significant reduced AUC calculated from EAE scores (FIG. 2). Five (5) mice died in each of these groups. IMCY-0189 m1Ψ treatment also induced a statistically significant MMS reduction as compared to the vehicle control group. MOG₃₅₋₅₅ m1Ψ treatment did not induce any statistically significant MMS reduction. We conclude that IMCY-0189 m1Ψ outperforms MOG₃₅₋₅₅ m1Ψ in mouse EAE treatment.

1. A non-immunogenic RNA encoding a peptide, said peptide comprising:
a) an oxidoreductase motif;
b) a T-cell epitope of an antigenic protein; and
c) a linker between a) and b) of between 0 and 7 amino acids, preferably of between 0 and 4 amino acids;
wherein said oxidoreductase motif a) comprises the following general structure:
[CST]XXC— or CXX[CST]—(SEQ ID NO: 1 or 2),
wherein X is any amino acid;
wherein [CST] indicates a single serine, threonine or cysteine residue;
wherein the hyphen (-) in said oxidoreductase motif indicates the point of attachment of the oxidoreductase motif to the N-terminal end of the linker or the T cell epitope, or to the C-terminal end of the linker or the T cell epitope.

2. The non-immunogenic RNA of claim 1, wherein the first amino acid of the encoded peptide is a methionine.

3. The non-immunogenic RNA of claim 1, wherein the non-immunogenic RNA is rendered non-immunogenic by the incorporation of modified nucleotides and the removal of dsRNA.

4. The non-immunogenic RNA of claim 3, wherein the modified nucleotides comprise a replacement of one or more uridines with a nucleoside comprising a modified nucleobase, preferably wherein the modified nucleobase is a modified uracil.

5. The non-immunogenic RNA of claim 1, wherein the nucleoside comprising a modified nucleobase is selected from the group consisting of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (tm5U), 1-taurinomethyl-pseudouridine, 5-taurinomethyl-2-thio-uridine (tm5s2U), 1-taurinomethyl-4-thio-pseudouridine, 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N(1)-methyl-pseudouridine (ml), 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (Wm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine, preferably wherein the nucleoside comprising a modified nucleobase is pseudouridine (ψ), N(1)-methyl-pseudouridine (m1ψ) or 5-methyl-uridine (m5U).

6. The non-immunogenic RNA of claim 1, wherein the nucleoside comprising a modified nucleobase is N(1)-methyl-pseudouridine (m1ψ).

7. The non-immunogenic RNA of claim 1, wherein the non-immunogenic RNA is formulated in a delivery vehicle, preferably wherein the delivery vehicle comprises particles, a lipid, or a cationic lipid.

8. The non-immunogenic RNA of claim 7, wherein the lipid forms a complex with and/or encapsulates the non-immunogenic RNA, or wherein the non-immunogenic RNA is formulated in liposomes.

9. The non-immunogenic RNA of claim 1, wherein said oxidoreductase motif comprises a sequence defined by any one of SEQ ID NOs: 24 to 35.

10. The non-immunogenic RNA of claim 1, encoding an immunogenic peptide that has a length of between 9 and 50 amino acids, preferably of between 9 and 30 amino acids.

11. The non-immunogenic RNA of claim 1, wherein the oxidoreductase motif in said immunogenic peptide does not naturally occur in the amino acid sequence within a region of 11 amino acids N-terminally or C-terminally of the T-cell epitope in said antigenic protein and/or wherein said the T-cell epitope does not naturally comprise said oxidoreductase motif in its amino acid sequence.

12. The non-immunogenic RNA according to claim 1, wherein said T cell epitope is an MHC class II T cell epitope having a length of between 7 and 25 amino acids, preferably between 9 and 25 amino acids; or wherein said T cell epitope in the peptide is an NKT cell epitope having a length of between 7 and 25 amino acids.

13. A pharmaceutical composition comprising the non-immunogenic RNA according to claim 1 and optionally a pharmaceutically acceptable excipient.

14. (canceled)

15. A method of treating or preventing a disease or condition selected from an autoimmune disease, an infection with an intracellular pathogen, a tumor, an allograft rejection, or an immune response to a soluble allofactor, to an allergen exposure or to a viral vector used for gene therapy or gene vaccination in a subject, comprising administering to the subject the non-immunogenic RNA according to claim 1.

16. (canceled)

17. A method of inducing cytolytic CD4+ T cells in a subject, comprising administering to the subject non-immunogenic RNA encoding the peptide according to claim 1.

18. (canceled)

19. The method of claim 15, wherein the subject has an autoimmune disease.

20. The method of claim 19, wherein the autoimmune disease is selected from the group comprising: type-1 diabetes (T1D), a demyelinating disorder such as multiple sclerosis (MS) or neuromyelitis optica (NMO), or in rheumatoid arthritis (RA).

21. The method of claim 19, wherein
(a) said antigenic protein is selected from the group consisting of: (pro)insulin, GAD65, GAD67, IA-2 (ICA512), IA-2 (beta/phogrin), IGRP, Chromogranin, ZnT8 and HSP-60, and wherein said autoimmune disease is type-1 diabetes (T1D);
(b) said antigenic protein is selected from the group consisting of: Myelin oligodendrocyte glycoprotein (MOG), Myelin basic protein (MBP), Proteolipid protein (PLP), Myelin-oligodendrocytic basic protein (MOBP), and Oligodendrocyte-specific protein (OSP), and wherein said autoimmune disease is multiple sclerosis (MS), and/or neuromyelitis optica (NMO); or
(c) said antigenic protein is selected from the group consisting of: GRP78, HSP60, 60 kDa chaperonin 2, Gelsolin, Chitinase-3-like protein 1, Cathepsin S, Serum albumin, and Cathepsin D, and wherein said autoimmune disease is rheumatoid arthritis (RA).

22-23. (canceled)

24. The method of claim 15, wherein said antigenic protein is a tumor or cancer antigen such as: an oncogene, proto-oncogene, viral protein, surviving factor or clonotypic or idiotypic determinant, wherein said disease is cancer.

25. The method according to claim 21, wherein
(a) said non-immunogenic RNA encodes a peptide comprising a T-cell epitope of MOG, more preferably an MHC class II T cell epitope YRSPFSRVV (SEQ ID NO: 704), FLRVPCWKI (SEQ ID NO: 4) or FLRVPSWKI (SEQ ID NO: 5);
(b) said non-immunogenic RNA is defined by SEQ ID NO. 778;
(c) the peptide encoded by said non-immunogenic RNA is defined by SEQ ID NO. 777 or 707; or
(d) said non-immunogenic RNA encodes a peptide comprising a T-cell epitope of insulin, preferably comprising the MHC class II T cell epitope LALEGSLOK (SEQ ID NO: 3).

26-28. (canceled)
